SEQUENCE LISTING (8 sequences)

SEQ ID NO: 1 - protein, 427 aa, Homo Sapiens
STQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 2 - DNA, 194 bp, Homo Sapiens
agatctaaaaaaaaaaagaagaagaagaagaagaaaaaaaaaaaaaagaaaaagaaaaagaagaaaaagaaaaaaaagaagaaaaagaagaaaaagaaaaagaaaaagaaaaagaaaaagaagaaaaagaaaaagaaaaagaagaaaaagaaaaagaaaaagaaaaagaaaaagaaaaagaaataggcggccgc

SEQ ID NO: 3 - DNA, 194 bp, Homo Sapiens
agatctcgtaggaaacgcaagcgaaaacggaagcgcaggaaacggaagcgtcgcaagaaacgacggaagagaaagaggcgtaagaagcgcaaacgaaagcggaaaaggaagcggagaaagaagcgacgtaagcgcaaacgtcgcaaacgtagaaaacgtcgaaagcgtaaacgcaagcgtaaataggcggccgc

SEQ ID NO: 4 - protein, 10 aa, Artificial Sequence
  HIV tat peptide sequence
GRKKRRQRRR

SEQ ID NO: 5 - protein, 7 aa, Artificial Sequence
  NLS peptide sequence
PKKKRKV

SEQ ID NO: 6 - protein, 17 aa, Artificial Sequence
  tat-NLS peptide sequence
GRKKRRQRRRPKKKRKV

SEQ ID NO: 7 - DNA, 1445 bp, Homo Sapiens
tccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatgacgtactcctgcctccctccctcccagggctccatccagctgtgcagtggggaggactggccagaccttctgtccactgttgcaatgaccccaggaagctacccccaataaactgtgcctgctcagagccccagtacacccattcttgggagcgggcagggc

SEQ ID NO: 8 - protein, 320 aa, Homo Sapiens
FTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

(19) United States
(12) Patent Application Publication
(10) Pub. No.: US 2011/0020373 A1
(43) Pub. Date: Jan. 27, 2011

(54) IGE DIRECTED DNA VACCINATION

(75) Inventors: Andrew Saxon, Santa Monica, CA (US); Ke Zhang, Los Angeles, CA (US)

Correspondence Address: ARNOLD & PORTER LLP;ATTN: IP DOCKETING DEPT., 555 TWELFTH STREET, N.W., WASHINGTON, DC, 20004-1206, US

(73) Assignee: THE REGENTS OF THE UNIVERSITY OF CALIFORNIA, Oakland, CA (US)

(21) Appl. No.: 12/294,852

(22) PCT Filed: Mar. 30, 2007
(86) PCT No.: PCT/US2007/008028

§ 371 Date: Sep. 26, 2008

Related U.S. Application Data
(60) Provisional application No. 60/788,457, filed on Mar. 30, 2006.
(60) Provisional application No. 60/848,545, filed on Sep. 29, 2006.

(51) Int. Cl. A61K 39/395; A61P 31/12; A61P 37/00; A61P 35/04

(52) U.S. Cl. 424/181.1; 424/178.1

(57) ABSTRACT

This invention is directed to a novel approach for focusing and expressing DNA vaccinates in Antigen Presenting Cells (APCs) mediated through targeting IgE receptors (FcεRs) on APC and driving DNA expression through provision of an APC specific regulatory element This vaccine can be used in the prevention or treatment of allergic disease.

### STATEMENT REGARDING FEDERALLY SPONSORED RESEARCH OR DEVELOPMENT

This invention was made with government support under Grant No. AI15251 awarded by the National Institutes of Health. The Government has certain rights in this invention.

### TECHNICAL FIELD

The invention concerns a novel approach for focusing and expressing DNA vaccines in Antigen Presenting Cells (APCs) mediated through targeting IgE receptors (FcεRs) on APCs and driving DNA expression through provision of an APC specific regulatory element. Such improved DNA vaccines are useful in the management of IgE-mediated allergic diseases and other disorders, eg. autoimmune disorders, infectious diseases such are viral diseases and cancer where DNA vaccination is expected to have a beneficial effect.

### BACKGROUND OF THE INVENTION

Immunoglobulin receptors (also referred to as Fc receptors) are cell-surface receptors binding the constant region of immunoglobulins, and mediate various immunoglobulin functions other than antigen binding. Fc receptors for IgE molecules are found on many cell types of the immune system (Fridman, W., FASEB J, 5(12):2684-90 (1991)). There are two different receptors currently known for IgE. IgE mediates its biological responses as an antibody through the multichain high-affinity receptor, FcεRI, and the low-affinity receptor, FcεRII. The high-affinity FcεRI, expressed on the surface of mast cells, basophils, dendritic cells, monocytes, macrophages and Langerhans cells, belongs to the immunoglobulin gene superfamily, and has a tetrameric structure composed of an ε-chain, a β-chain and two disulfide-linked γ-chains (αβγ2) in mast cells and basophils, and an αγ2 structure in dendritic cells, monocytes, macrophages and Langerhans cells (Adamczewski, M., and Kinet, J. P., Chemical Immun., 59:173-190 (1994)) that are required for receptor expression and signal transduction (Tunon de Lara, Rev. Mal. Respir., 13(1):27-36 (1996)). The ε-chain of the receptor interacts with the distal portion of the third constant domain of the IgE heavy chain. The specific amino acids of human IgE involved in binding to human FcεRI have been identified as including Arg-408, Ser-41 1, Lys-415, Glu-452, Arg-465, and Met-469 (Presta et al., J. Biol. Chem. 269:26368-73 (1994)). The interaction is highly specific with a binding constant of about 10¹⁰ M⁻¹.

The low-affinity FcεRII receptor (CD23), represented on the surface of inflammatory cells, including eosinophils, leukocytes, B lymphocytes, and platelets, did not evolve from the immunoglobulin superfamily but has substantial homology with several animal lectins (Yodoi et al., Ciba Found. Symp., 147:133-148 (1989)) and is made up of a transmembrane chain with an intracytoplasmic NH₂ terminus. FcεRII is currently known to have two forms (FcεRIIa and FcεRIIb), both of which have been cloned and sequenced. They differ only in the N-terminal cytoplasmic region, the extracellular domains being identical. FcεRIIa is normally expressed on B cells, while FcεRIIb is expressed on T cells, B cells, monocytes and eosinophils upon induction by the cytokine IL-4.

Through the high-affinity IgE receptor, FcεRI, IgE plays key roles in an array of acute and chronic allergic reactions, including asthma, allergic rhinitis, atopic dermatitis, severe food allergies, chronic urticaria and angioedema, as well as the serious physiological condition of anaphylactic shock as results, for example, from bee stings or penicillin allergy. Binding of a multivalent antigen (allergen) to antigen specifically bound to FcεRI on the surface of mast cells and basophils stimulates a complex series of signaling events that culminate in the release of host vasoactive and proinflammatory mediators contributing to both acute and late-phase allergic responses (Metcalfe et al., Physiol. Rev. 77:1033-1079 (1997)).

The function of the low affinity IgE receptor, FcεRII found on the surface of B lymphocytes, is much less well established than that of FcεRI. FcεRII, in a polymeric state, binds IgE, and this binding may play a role in controlling the type (class) of antibody produced by B cells.

Human antigen presenting cells (APCs) including macrophages/monocytes, blood dendritic cells (DC), follicular DC (FDC), Langerhans' cells (LC), mast cells and activated B cells, differentially express FcεRI and/or FcεRII. It has been demonstrated that antigens can be efficiently captured by APCs via IgE Dependent Antigen Focusing (IgE-DAF) pathways and presented directly to B cells, or processed and presented to T cells to elicit heightened immune responses.

Targeting of antigen-IgE complexes to FcεRI-bearing peripheral blood dendritic cells has been shown to result in a much stronger antigen-specific T cell response than that elicited following dendritic cell exposure to antigen in the absence of IgE (Maurer et al., 1995 J. Immunol. 154:6258; Maurer et al., 1998, J. Immunol. 161:2731). Antigen taken up by dendritic cells via the FcεRI is efficiently internalized into MHC-containing compartments, where the antigen is then processed and loaded onto MHC through a cathepsin S-dependent pathway (Maurer et al., 1998 J. Immunol. 161:2731). Other types of DC, such as FDC, epidermal Langerhans' cells and dermal DC, also express FcεRI, and the FcεRI expressed on these types of APCs is thought to play an important role via IgE-DAF and presentation under specific circumstances and in special locations (Mudde et al., 1990 Immunol Today 11:440). For example, IgE-mediated capture and presentation of antigens in FDC is a mechanism that may provide for long-lasting immune responses due to the ability of FDC to maintain antigens on their surface for prolonged periods of time, and specialized localization and interaction with T cells in germinal centers of the lymphoid tissues (Mudde et al., 1990 Immunol Today 11:440). Such an IgE-DAF mechanism is particularly important when the concentration of a given antigen is below the concentration that can be effectively presented through conventional antigen capture and presentation pathways. The extraordinary high affinity of the FcεRI for the Fc region of IgE (Kd between 10⁻¹⁰ to 10⁻¹¹ L/M range), an affinity 2 to 3 logs higher than most ligand-receptor interactions, likely accounts for the special place this interaction has in enhancing antigen presentation.

Studies of IgE-DAF mediated by FcεRII (CD23) B cells also indicate that IgE-mediated antigen capture with subsequent processing and presentation are 2-3 log fold more effective than that in the absence of antigen-specific IgE (Mudde et al., 1990 Immunol Today 11:440; Kehry et al., 1989 Proc. Natl. Acad. Sci. USA 86:7556; Pirron et al., 1990 Eur. J. Immunol. 20:1547). Such IgE mediated enhancement of antigen presentation activity was shown to be both IgE-dependent and IgE specific, as antigen specific IgG did not show the same effects, and the IgE-DAF did not present bystander antigens (Saxon et al., 2001 The Allergic Response in Host Defense. In Clinical Immunology Rich R. R. et al., (eds) 2^{nd} edition pp 451). Some types of APCs such as FDCs are likely able to capture and present antigens through both FcεRI and FcεRII as they express both types of FcεRs. (Mudde et al., 1990 Immunol Today 11:440; Saxon et al., 2001 The Allergic Response in Host Defense. In Clinical Immunology Rich R. R. et al., (eds) 2^{nd} edition pp 451).

Despite advances in understanding the cellular and molecular mechanisms that control allergic responses and improved therapies, the incidence of allergic diseases, especially asthma and severe food allergy, has increased dramatically in recent years in both developed and developing countries (Beasley et al., J. Allergy Clin. Immunol. 105:466-472 (2000); Peat and Li, J. Allergy Clin. Immunol. 103:1-10 (1999). Ma et al., J Allergy Clin Immunol. 112:784-8 (2003)).

Through the high-affinity IgE receptor FcεRI, IgE plays key roles in immune response. The activation of mast cells and basophils by antigen (i.e., allergen) via an antigen-specific IgE/FcεRI pathway results in the release of host vasoactive and proinflammatory mediators (i.e., degranulation), which contributes to the allergic response (Oliver et al., Immunopharmacology 48:269-281 (2000); Metcalfe et al., Physiol: Rev., 77:1033-1079 (1997)). These and other biochemical events lead to the rapid secretion of inflammatory mediators such as histamine, resulting in physiological responses that include localized tissue inflammation, vasodilation, increased blood vessel and mucosal permeability, and local recruitment of other immune system cells, including additional basophils and mast cells. In moderation, these responses have a beneficial role in immunity against parasites and other microorganisms. However, when in excess, this physiological response results in the varied pathological conditions of allergy, also known as type I hypersensitivity.

Allergy is manifested in a broad array of conditions and associated symptoms, which may be mild, chronic, acute and/or life threatening. These various pathologies include, for example, allergic asthma, allergic rhinitis, atopic dermatitis, severe food allergies, chronic urticaria and angioedema, as well as the serious physiological condition of anaphylactic shock. A wide variety of antigens are known to act as allergens, and exposure to these allergens results in the allergic pathology. Common allergens include, but are not limited to, bee stings, penicillin, various food allergies, pollens, animal proteins (especially house dust mite, cat, dog and cockroach), and fungal allergens. The most severe responses to allergens can result in airway constriction and anaphylactic shock, both of which are potentially fatal conditions. Despite advances in understanding the cellular and molecular mechanisms that control allergic responses and improved therapies, the incidence of allergic diseases, especially allergic asthma, has increased dramatically in recent years in both developed and developing countries (Beasley et al., J. Allergy Clin. Immunol. 105:466-472 (2000); Peat and Li, J. Allergy Clin. Immunol. 103:1-10 (1999)). Thus, there exists a strong need to develop treatments for allergic diseases.

Allergic asthma is a condition brought about by exposure to ubiquitous, environmental allergens, resulting in an inflammatory response and constriction of the upper airway in hypersensitive individuals. Mild asthma can usually be controlled in most patients by relatively low doses of inhaled corticosteroids, while moderate asthma is usually managed by the additional administration of inhaled long-acting β-antagonists or leukotriene inhibitors. The treatment of severe asthma is still a serious medical problem. In addition, many of the therapeutics currently used in allergy treatment have serious side-effects. Although an anti-IgE antibody currently in clinical use (rhuMAb-E25, Genentech, Inc.) and other experimental therapies (e.g., antagonists of IL-4) show promising results, there is need for the development of additional therapeutic strategies and agents to control allergic disease, such as asthma, severe food allergy, and chronic urticaria and angioedema.

Allergic diseases can be treated, for example, by allergen-based vaccination, in which increasing doses of allergen are given by injection over years. This approach is costly, time consuming, poorly or not efficacious in many allergic conditions, and has serious side-effects, including death in some instances. One approach to the treatment of allergic diseases is by use of allergen-based immunotherapy. This methodology uses whole antigens as “allergy vaccines” and is now appreciated to induce a state of relative allergic tolerance. This technique for the treatment of allergy is frequently termed “desensitization” or “hyposensitization” therapy. In this technique, increasing doses of allergen are administered, typically by injection, to a subject over an extended period of time, frequently months or years. The mechanism of action of this therapy is thought to involve induction of IgG inhibitory antibodies, suppression of mast cell/basophil reactivity, suppression of T-cell responses, the promotion of T-cell anergy, and/or clonal deletion, and in the long term, decrease in the levels of allergen specific IgE. The use of this approach is, however, hindered in many instances by poor efficacy and serious side-effects, including the risk of triggering a systemic and potentially fatal anaphylactic response, where the clinical administration of the allergen induces the severe allergic response it seeks to suppress (TePas et al., Curr. Opin. Pediatrics 12:574-578 [2000]).

Refinements of this technique use smaller portions of the allergen molecule, where the small portions (i.e., peptides) presumably contain the immunodominant epitope(s) for T cells regulating the allergic reaction. Immunotolerance therapy using these allergenic portions is also termed peptide therapy, in which increasing doses of allergenic peptide are administered, typically by injection, to a subject. The mechanism of action of this therapy is thought to involve suppression of T-cell responses, the promotion of T-cell anergy, and/or clonal deletion. Since the peptides are designed to bind only to T cells and not to allergic (IgE) antibodies, it was hoped that the use of this approach would not induce allergic reactions to the treatment. Unfortunately, these peptide therapy trials have met with disappointment, and allergic reactions are often observed in response to the treatments. Development of these peptide therapy methods have largely been discontinued.

Allergic responses are strongly associated with Th2 type immune responses. Modulation of the skewed Th2 response toward a more balanced response is the major goal of the allergen immunotherapy isorders including asthma. To this end, protein-based allergen immunotherapy has been widely used in clinical practice. However, the efficacy of such allergen immunotherapy is variable, a long duration (several years) of treatment is required, and more importantly, allergen immunotherapy can unpredictably trigger local and systemic allergic responses. There are no reliable ways to forecast whether an allergen immunotherapy will trigger allergic responses and immunotherapy may be particularly dangerous in severe allergic asthma and other life-threatening allergic conditions.

Administering allergen genes to patients has been demonstrated to be an effective approach for allergy immunotherapy (Raz, E., et al., 1996, Proc Natl Acad Sci USA. 93: 5141; Hsu, C. H., et al. (1996) Nat Med. 2:540; Hsu, C. H., et al (1996) Int Immunol. 8:1405; Lee, D. L., et al. (1997) Int Arch Allergy Immunol. 113:227; Slater, J. E., et al. (1998), J Allergy Clin Immunol. 102:469; Li, X., et al. (1999), J. Immunol. 162:3045; Toda, M., et al. (2000). Immunology. 99: 179; Maecker, H. T., et al. (2001). J Immunol. 166:959; Jilek, S., et al. (2001) J Immunol. 166:3612; Hochreiter, R., et al. (2001), Int Arch Allergy Immunol. 124: 406; Adel-Patient, K., et al. (2001), Int Arch Allergy Immunol. 126:59; Peng, H. J., et al. (2002), Vaccine. 20: 1761; Bauer, R., et al. (2003) Allergy. 58:1003; Wolfowicz, C. B., et al (2003) Vaccine. 21:1195; Jacquet, A et al. (2003) Clin Exp Allergy. 33:218; Chatel, J. M., et al. (2003) Allergy. 58:641; Sudowe, S., et al. (2003) Mol Ther. 8: 567; Toda, M., et al (2002) Eur J. Immunol. 32:1631; Hochreiter, R., et al. (2003) Eur J. Immunol. 33:1667; Roy, K., et al. 1999. Nat. Med. 5:387; Chew, J C., et al. 2003. Vaccine. 21:2720; Sudowe, S., et al. 2002. Gene Ther. 9:147; Ludwig-Portugall, I et al. 2004. J Allergy Clin Immunol. 114:951; Sudowe, S., et al. 2006. J Allergy Clin Immunol. 117:196-203).

Allergen gene vaccination represents a promising alternative to the protein-based immunotherapy protocols for allergen-specific immunotherapy in terms of safety concern and efficacy, as this approach has been shown to be safe and effectively inhibit allergen-specific IgE production, suppress Th2 response, and reciprocally enhance Th1 response. When effective, allergen vaccination has achieved more balanced Th2/Th1 responses, including suppression of Th2 responses and IgE production, and enhancement of IFN-γ, IgG2a and Th1 responses (Darcan, Y., et al., Vaccine 23:4203). In addition, the allergen gene-based vaccination also could reduce the numbers of mast cells in allergic inflammation sites such as the lung (Masuda K. (2005). Vet Immunol Immunopathol. 108:185). Allergen genes have been administered as naked plasmid DNA by various routes, including intramuscular or intradermal injection, biolistic transfection via the gene gun, or orally as plasmid DNA-polymer complexes. DNA immunization by injection has been reported to be effective in inhibiting development of specific IgE production. In contrast, the ability of DNA vaccination with allergen-encoding vectors to suppress already established IgE immune responses is controversial. A major hurdle for effective allergen gene therapy has been the poor efficiency of DNA transfer and expression in allergic disease models.

### Autoimmune Diseases

It is estimated that as much as 20 percent of the American population has some type of autoimmune disease. Autoimmune diseases demonstrate disproportionate expression in women, where it is estimated that as many as 75% of those affected with autoimmune disorders are women. Although some forms of autoimmune diseases are individually rare, some diseases, such as rheumatoid arthritis and autoimmune thyroiditis, account for significant morbidity in the population (Rose and MacKay (Eds.), The Autoimmune Diseases, Third Edition, Academic Press [1998]).

Autoimmune disease results from failure of the body to eliminate self-reactive T-cells and B-cells from the immune repertoire, resulting in circulating B-cell products (i.e., autoreactive antibodies) and T-cells that are capable of identifying and inducing an immune response to molecules native to the subject's own physiology. Particular autoimmune disorders can be generally classified as organ-specific (i.e., cell-type specific) or systemic (i.e., non-organ specific), but with some diseases showing aspects of both ends of this continuum. Organ-specific disorders include, for example, Hashimoto's thyroiditis (thyroid gland) and insulin dependent diabetes mellitus (pancreas). Examples of systemic disorders include rheumatoid arthritis and systemic lupus erythematosus. Since an autoimmune response can potentially be generated against any organ or tissue in the body, the autoimmune diseases display a legion of signs and symptoms. Furthermore, when blood vessels are a target of the autoimmune attack as in the autoimmune vasculitides, all organs may be involved. Autoimmune diseases display a wide variety of severity varying from mild to life-threatening, and from acute to chronic, and relapsing (Rose and MacKay (Eds.), The Autoimmune Diseases, Third Edition, Academic Press [1998]; and Davidson and Diamond, N. Engl. J. Med., 345(5):340-350 [2001]).

The molecular identity of some of the self-reactive antigens (i.e., the autoantigen) are known in some, but not all, autoimmune diseases. The diagnosis and study of autoimmune diseases is complicated by the promiscuous nature of these disorders, where a patient with an autoimmune disease can have multiple types of autoreactive antibodies, and vice versa, a single type of autoreactive antibody is sometimes observed in multiple autoimmune disease states (Mocci et al., Curr. Opin. Immunol., 12:725-730 [2000]; and Davidson and Diamond, N. Engl. J. Med., 345(5):340-350 [2001]). Furthermore, autoreactive antibodies or T-cells may be present in an individual, but that individual will not show any indication of disease or other pathology. Thus while the molecular identity of many autoantigens is known, the exact pathogenic role of these autoantigens generally remains obscure (with notable exceptions, for example, myesthenia gravis, autoimmune thyroid disease, multiple sclerosis and diabetes mellitus).

Treatments for autoimmune diseases exist, but each method has its own particular drawbacks. Existing treatments for autoimmune disorders can be generally placed in two groups. First, and of most immediate importance, are treatments to compensate for a physiological deficiency, typically by the replacement of a hormone or other product that is absent in the patient. For example, autoimmune diabetes mellitus can be treated by the administration of insulin, while autoimmune thyroid disease is treated by giving thyroid hormone. Treatments of other disorders entails the replacement of various blood components, such as platelets in immune thrombocytopenia or use of drugs (e.g., erythropoetin) to stimulate the production of red blood cells in immune based anemia. In some cases, tissue grafts or mechanical substitutes offer possible treatment options, such as in lupus nephritis and chronic rheumatoid arthritis. Unfortunately, these types of treatments are suboptimal, as they merely alleviate the disease symptoms, and do not correct the underlying autoimmune pathology and the development of various disease related complications. Since the underlying autoimmune activity is still present, affected tissues, tissue grafts, or replacement proteins are likely to succumb to the same immune degeneration.

DCs as professional APCs are crucial for the initiation of transgene-specific immune responses for all methods of DNA delivery (Takashima A. and Morita, A. (1999) J Leukoc Biol. 66: 350). However, none of the current gene-transfer methods for allergen gene vaccination specifically targets the DNA gene to DCs. The resulting low efficiency of these approaches is likely related to the low efficiency of vaccine gene delivery to DCs.

In one aspect, this invention is directed to a better way to enhance the efficiency of the allergen gene vaccination to specifically target allergen genes to DCs. The extremely high affinity interaction between IgE and FcεRI provides a unique feature that could be utilized for the development of such an efficient allergen gene delivery platform for allergen IT. Such a possibility is especially suitable for allergen gene-based IT for atopic patients, as APCs of the allergic patients, particularly in DCs and Langerhans cells, express much higher levels of FcεRI than those in non-allergic individuals (Mudde, G. C., Hansel, T. T., and van Reijsen, F. C. (1990). Immunol Today. 11:440; Haas, N., et al., (1992). Acta Derm Venereol. 72:271; Grabbe, J., et al., (1993). Br J Dermatol. 129:120; Haas, N., et al., (1993) Exp Dermatol. 2:157. Maurer, D., et al., (1994). J. Exp Med. 179:745; Allam, J. P., et al., (2003) J. Allergy. Clin. Immunol. 112:141; Bieber T, et al., (1992) J Exp Med. 175:1285). This unique feature ensures that IgE-mediated allergen gene transfer specifically targeting DCs could be much more efficiently achieved for allergen vaccination in allergic patients. Thus, the current proposal is designed to overcome a major obstacle to the successful use of allergen gene vaccination in humans.

The object of this invention is to provide an improved vaccine for focusing and expressing DNA vaccinates in Antigen Presenting Cells (APCs) mediated through targeting IgE receptors (FcεRs) on APC and driving DNA expression through provision of an APC specific regulatory element. Such improved DNA vaccines will be useful in the management of IgE-mediated allergic diseases and other disorders, eg. autoimmune disorders, infectious diseases such are viral diseases and cancer where DNA vaccination is expected to have a beneficial effect.

### BRIEF SUMMARY OF THE INVENTION

The present invention provides for novel vaccines for focusing and expressing DNA in Antigen Presenting Cells (APCs) mediated through targeting IgE receptors (FcεRs) on APCs and driving DNA expression through provision of an APC specific regulatory element, as well as methods for using such compounds, and compositions and articles of manufacture comprising them. The invention also provides compositions and methods suitable for the prevention or treatment of immune-mediated diseases.

One aspect of the invention concerns a composition for delivering DNA vaccines to dendritic cells comprising an IgE peptide capable of binding to a native IgE receptor functionally linked to a nucleic acid binding agent.

Another aspect of the invention is directed to a composition comprising an IgE peptide capable of binding to an IgE receptor functionally linked to a nucleic acid binding agent which is directly or indirectly linked to a DNA vaccine.

Another aspect of the invention concerns a vaccine comprising a nucleic acid encoding an allergen functionally connected to an IgE fragment capable of binding a native Fce receptor. In one embodiment the nucleic acid is indirectly functionally connected to the IgE fragment.

In another embodiment, the IgE fragment or peptide sequence comprises preferably an amino acid sequence having at least 85% identity to the CH2-CH3-CH4 domain amino acid sequence of SEQ ID NO: 1, and more preferably, at least 90% identity, and more preferably still, at least 95% identity, and most preferably, at least 98% identity. In still other embodiments, the IgE fragment or peptide sequence comprises a least part of the CH2 and CH3 domains of a native human IgE constant region. Alternatively, the IgE fragment or peptide sequence comprises an amino acid sequence encoded by a nucleic acid hybridizing under stringent conditions to at least a portion of the complement of the IgE heavy chain constant region nucleotide sequence of SEQ ID NO: 1

In one aspect of the invention the nucleic acid is connected to the IgE fragment by a nucleic acid binding agent. In one embodiment the nucleic acid binding agent is selected from the group comprising repeated lysines, repeated lysines and arginines, spermine or spermidine, or polyethylimine polymer. In another aspect, the nucleic acid binding agent comprises poly-l-lysine. In another aspect, the nucleic acid binding agent comprises poly-l-lysine-arginine. In another aspect of the invention the IgE fragment is attached to the nucleic acid binding agent by a linkage selected from the group consisting of a covalent bond, a disulfide bond or an avidin/streptavidin linkage.

In another aspect the IgE fragment or peptide comprises the CH2-CH3-CH4 domains of IgE or the CH1-CH2-CH3-CH4 domains of IgE. In one embodiment the IgE fragment or peptide is human.

In all aspects, the DNA vaccine or nucleic acid encoding the allergen may be operably linked to a dendritic cell promoter. The dendritic cell promoter may be the fascin promoter. In one embodiment the nucleic acid comprises a vector.

In another embodiment the allergen DNA sequence encodes an allergen is selected from the group of allergens described in Table 1. In one embodiment the allergen DNA is Fel d1. In another embodiment the allergen DNA is that for Ara h1 from peanuts. In other embodiments, the DNAs comprise a mixture of those encoding the major peanut allergens (Ara h1-6).

In other preferred embodiments, the antigen nucleic acid sequence comprises at least 90% sequence identity with at least a portion of an antigen nucleic acid sequence. In still other preferred embodiments, the antigen nucleic acid sequence comprises an nucleic acid sequence which hybridizes under stringent conditions to at least a portion of the complement of a nucleic acid molecule encoding an antigen.

In another embodiment the DNA sequence is that for an immunogen derived from an infectious agent. In another embodiment the DNA sequence is that for an immunogen derived from a cancer cell. In another embodiment, the DNA sequence is that for an immunogen that is a self antigen, e.g. an autoantigen.

In another embodiment the autoantigen DNA sequence encodes an autoantigen is selected from the group of autoantigens described in Table 2. In some preferred embodiments, the autoantigen DNA sequence encodes an autoantigen sequence selected from the group consisting of rheumatoid arthritis autoantigen, multiple sclerosis autoantigen, or autoimmune type I diabetes mellitus autoantigen, and portions thereof. In other preferred embodiments, the autoantigen DNA sequence encodes an autoantigen is selected from the group consisting of myelin basic protein (MBP), proteolipid protein, myelin oligodendrocyte glycoprotein, αβ-crystallin, myelin-associated glycoprotein, Po glycoprotein, PMP22, 2′,3′-cyclic nucleotide 3′-phosphohydrolase (CNPase), glutamic acid decarboxylase (GAD), insulin, 64 kD islet cell antigen (IA-2, also termed ICA512), phogrin (IA-2β), type II collagen, human cartilage gp39 (1-ICgp39), and gp130-RAPS, and portions thereof.

In other preferred embodiments, the autoantigen nucleic acid sequence comprises at least 90% sequence identity with at least a portion of an autoantigen nucleic acid sequence. In still other preferred embodiments, the autoantigen nucleic acid sequence comprises an nucleic acid sequence which hybridizes under stringent conditions to at least a portion of the complement of a nucleic acid molecule encoding an autoantigen.

Another aspect of the invention is a pharmaceutical composition comprising a vaccine of the invention in admixture with a pharmaceutically acceptable ingredient.

Another aspect of the invention is an article of manufacture comprising a container, the vaccine of the invention within the container, and a label or package insert on or associated with the container. In one embodiment the label or package insert comprises instructions for the treatment of an IgE-mediated biological response. In one embodiment the biological response is an IgE-mediated hypersensitivity reaction. In one embodiment the label or package insert comprises instruction for the treatment of a condition selected from the group consisting of asthma, allergic rhinitis, atopic dermatitis, severe food allergies, chronic urticaria, angioedema, and anaphylactic shock.

Another aspect of the invention is a method for the prevention or treatment of a condition associated with an IgE-mediated biological response, comprising administering an effective amount of a vaccine of the invention to a subject in need. In one embodiment the subject is a human patient. In one embodiment the condition is IgE-mediated hypersensitivity reaction. In one embodiment the condition is selected from the group consisting of asthma, allergic rhinitis, atopic dermatitis, severe food allergies, chronic urticaria, angioedema, and anaphylactic shock.

In another aspect, the invention provides a method for the treatment or prevention of symptoms resulting from a type I hypersensitivity reaction in a subject comprising administering at least one vaccine of the present invention to the subject. In another embodiment, the type I hypersensitivity reaction is an anaphylactic response. In another embodiment of this method, the type I hypersensitivity symptoms being prevented comprise an anaphylactic response.

In various embodiments of this method, the vaccine is administered to the subject prior to the onset of the biological response or during the biological response.

It is contemplated that vaccine of this invention may be administered with other vaccines or treatments such as local or systemic use of biological response modifiers.

These and other aspects of the invention will become more evident upon reference to the following detailed description and attached drawings. It is to be understood however that various changes, alterations and substitutions may be made to the specific embodiments disclosed herein without departing from their essential spirit and scope. In addition, it is further understood that the drawings are intended to be illustrative and symbolic representations of an exemplary embodiment of the present invention and that other non-illustrated embodiments are within the scope of the invention.

### BRIEF DESCRIPTION OF THE DRAWINGS

FIG. 1 shows a diagram of the IgE-mediated gene targeting to FcεRI expressing antigen presenting cells (APCs).

FIG. 2A is a diagram of the experimental schedule for Example 2.

FIG. 2B is a diagram of the experimental schedule for Example 3.

FIG. 3 are diagrams of the construction of the IgE-PLL and IgE-PRL fusion genes. The PLL DNA (SEQ ID NO:2) encodes 60 repeated lysines and the PRL DNA (SEQ ID NO:3) encodes 60 alternating lysines and arginines. The underlined sequences are the restriction sites used for cloning.

FIG. 4 is the construction, expression and characterization of EPL fusion protein. FIG. 4A is a diagram of construction of the EPL fusion protein. FIG. 4B is a Western blot of the EPL fusion protein under native (non-reduced) and reduced conditions. FIG. 4C is a picture of the gel retardation analysis of various proteins to plasmid. FIG. 1D is a gel retardation analysis of the effect of protein concentration on the binding of plasmid by EPL. FIG. 4E is a gel retardation analysis of the effect of nucleic acid concentration on the binding of plasmid by EPL. FIG. 4F is a graph of the FACS analysis of the binding of EPL-DNA to FcεR1 expressed on 3D10 cells. FIG. 4G is a graph of the FACS analysis of the binding of EPL-DNA to FcεR1 expressed on Ku812 cells. FIG. 4H is a picture of transgenic mice skin after passive curaneous anaphylaxis assay with EPL:DNA complex.

FIG. 5A is a picture of transgenic mice skin after administration of serum from human peanut allergic patients to mice and challenge with purified Ara h1 antigen. FIG. 5B is a picture of transgenic mice skin after administration of commercial serum from human peanut allergic patients to mice and challenge with purified Ara h1 antigen.

FIG. 6 is a diagram of the structure of the allergen gene vaccination plasmids using Ara h1 as an example.

FIG. 7 is a diagram of the modified EPLs structure with tat (SEQ ID NO:4), the NLS peptide (SEQ ID NO:5) or tat-NLS peptide (SEQ ID NO:6) sequences incorporated.

FIG. 8 is a schematic representation of the experimental design for testing EPL:allergen DNA plasmid polyplex effects in vivo.

FIG. 9 is a schematic diagram of the protocol for Example 4.

FIG. 10 is a schematic diagram of the protocol for Example 5.

FIG. 11 is a schematic diagram of the protocol for Example 6.

FIG. 12 is a schematic diagram of the protocol for combined Ara h1 gene vaccination as described in Example 6.

FIG. 13 shows the amino acid sequence encoding the human IgE heavy chain constant region (SEQ ID NO: 1).

FIG. 14 shows the nucleotide sequence of the human IgE heavy chain constant region (SEQ ID NO: 7).

FIG. 15 shows the amino acid sequence of the CH2-CH3-CH4 portion of the human IgE heavy chain constant region (SEQ ID NO: 8)

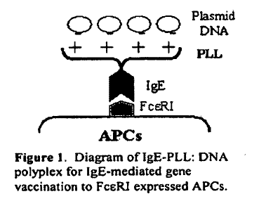

### DETAILED DESCRIPTION OF THE INVENTION

Unless defined otherwise, technical and scientific terms used herein have the same meaning as commonly understood by one of skill in the art to which this invention belongs.

One skilled in the art will recognize many methods and materials similar or equivalent to those described herein, which could be used in the practice of this invention. Indeed the present invention is no way limited to the methods and materials described herein. For purposes of the present invention the following terms are defined.

### DEFINITIONS

The term “functionally connected” with reference to the nucleic acid and the IgE fragment included in the vaccines herein, is used to indicate that the nucleic acid retains the ability to be transcribed and the IgE fragment retains the ability to bind to its receptor. Thus, after being connected to a nucleic acid sequence, the IgE fragment retains the ability of specific binding to a native high-affinity IgE receptor, e.g. native human FcεRI, or a native low-affinity IgE receptor, e.g. FcεRII, also known as CD23.

The binding is “specific” when the binding affinity of a molecule for a binding target, e.g. an IgG or IgE receptor, is significantly higher (at least about 2-times, at least about 4-times, or at least about 6-times higher) than the binding affinity of that molecule to any other known native polypeptide.

The term “native” or “native sequence” refers to a nucleic acid sequence or a polypeptide having the same nucleic acid sequence or amino acid sequence as a nucleic acid sequence or polypeptide that occurs in nature. A nucleic acid or polypeptide is considered to be “native” in accordance with the present invention regardless of its mode of preparation. Thus, such native sequence nucleic acid or polypeptide can be isolated from nature or can be produced by recombinant and/or synthetic means. The terms “native” and “native sequence” specifically encompass naturally-occurring truncated or secreted forms (e.g., an extracellular domain sequence), naturally-occurring variant forms (e.g., alternatively spliced forms) and naturally-occurring allelic variants of a polypeptide.

The terms “native FcεRI,” “native sequence FcεRI,” “native high-affinity IgE receptor FcεRI,” and “native sequence high-affinity IgE receptor FcεRI” are used interchangeably and refer to FcεRI receptors of any species, including any mammalian species, that occurs in nature. FcεRI is a member of the multi-subunit immune response receptor (MIRR) family of cell surface receptors that lack intrinsic enzymatic activity but transduce intracellular signals through association with cytoplasmic tyrosine kinases. For further details see, for example, Kinet, J. P., Annu. Rev. Immunol. 17:931-972 (1999) and Ott and Cambier, J. Allergy Clin. Immunol., 106:429-440 (2000).

The terms “native FcεRII”, “native sequence FcεRII”, native low-affinity IgE receptor FcεRII,” “native sequence low-affinity IgE receptor FcεRII” and “CD23” are used interchangeably and refer to FcεRII receptors of any species, including any mammalian species, that occur in nature. Several groups have cloned and expressed low-affinity IgE receptors of various species. The cloning and expression of a human low-affinity IgE receptor is reported, for example, by Kikutani et al., Cell 47:657-665 (1986), and Ludin et al., EMBO J. 6:109-114 (1987). The cloning and expression of corresponding mouse receptors is disclosed, for example, by Gollnick et al., J. Immunol. 144:1974-82 (1990), and Kondo et al., Int. Arch. Allergy Immunol 105:38-48 (1994). The molecular cloning and sequencing of CD23 for horse and cattle has been recently reported by Watson et al., Vet. Immunol. Immunopathol. 73:323-9 (2000). For an earlier review of the low-affinity IgE receptor see also Delespesse et al., Immunol. Rev. 125:77-97 (1992).

The term “immunoglobulin” (Ig) is used to refer to the immunity-conferring portion of the globulin proteins of serum, and to other glycoproteins, which may not occur in nature but have the same functional characteristics. The term “immunoglobulin” or “Ig” specifically includes “antibodies” (Abs). While antibodies exhibit binding specificity to a specific antigen, immunoglobulins include both antibodies and other antibody-like molecules that lack antigen specificity. Native immunoglobulins are secreted by differentiated B cells termed plasma cells, and immunoglobulins without any known antigen specificity are produced at low levels by the immune system and at increased levels by myelomas. As used herein, the terms “immunoglobulin,” “Ig,” and grammatical variants thereof are used to include antibodies, and Ig molecules without known antigen specificity, or without antigen binding regions.

Native immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{I}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The main mammalian Ig isotypes (classes) found in serum, and the corresponding Ig heavy chains, shown in parentheses, are listed below:

IgG (γ chain): the principal Ig in serum, the main antibody raised in response to an antigen, has four major subtypes, several of which cross the placenta;

IgE (ε chain): this Ig binds tightly to mast cells and basophils, and when additionally bound to antigen, causes release of histamine and other mediators of immediate hypersensitivity; plays a primary role in allergic reactions, including hay fever, asthma and anaphylaxis; may serve a protective role against parasites and may play an important role in antigen presentation;

IgA (α chain): this Ig is present in serum and particularly abundant in external secretions, such as saliva, tears, mucous, and colostrum;

IgM (μ chain): the Ig first induced in response to an antigen; it has lower affinity than antibodies produced later, is pentameric and primarily localized in the circulation; and

IgD (δ chain): this Ig is found in relatively high concentrations in umbilical cord blood, serves primarily as an early cell receptor for antigens and primarily functions as a lymphocyte cell surface molecule.

Antibodies of the IgG, IgE, IgA, IgM, and IgD isotypes may have the same variable regions, i.e. the same antigen binding cavities, even though they differ in the constant region of their heavy chains. The constant regions of an immunoglobulin, e.g. antibody are not involved directly in binding the antibody to an antigen, but correlate with the different effector functions mediated by antibodies, such as complement activation or binding to one or more of the antibody Fc receptors expressed on basophils, mast cells, lymphocytes, monocytes and granulocytes.

Some of the main human antibody isotypes (classes) are divided into further sub-classes. IgG has four known subclasses: IgG₁ (γ₁), IgG₂ (γ₂), IgG₃ (γ₃), and IgG4 (γ₄), while IgA has two known sub-classes: IgA₁ (α₁) and IgA₂ (α2).

A light chain of an Ig molecule is either a κ or a λ chain.

The constant region of an immunoglobulin heavy chain is further divided into globular, structurally discrete domains, termed heavy chain constant domains. For example, the IgE immunoglobulin heavy chain comprises four constant domains: CH1, CH2, CH3 and CH4 and does not have a hinge region.

Immunoglobulin sequences, including sequences of immunoglobulin heavy chain constant regions are well known in the art and are disclosed, for example, in Kabat et al., Sequences of Proteins of Immunological Interest, 5th Ed. Public Health Service, National Institute of Health, Bethesda, Md. (1991). For a discussion of the human IgG₁ heavy chain constant region (γ₁), see also Ellison et al., Nucl. Acid Res. 10:4071-4079 (1982); and Takahashi et al., Cell 29:671-679 (1982). For a discussion of the human IgE heavy chain constant region (c), see also Max et al., Cell 29:691-699 (1982). IgE isoforms are described in Saxon et al., J. Immunol. 147:4000 (1991); Peng et al., J. Immunol. 148:129-136 (1992); Zhang et al., J. Exp. Med. 176:233-243 (1992); and Hellman, Eur. J. Immunol. 23:159-167 (1992).

The terms “native IgE and “native sequence IgE”, are used interchangeably and refer to the IgE sequence of any species including any mammalian species, as occurring in nature. In one embodiment the animal is human.

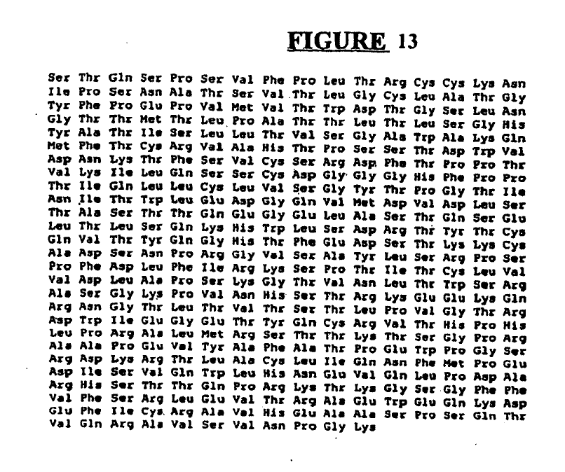

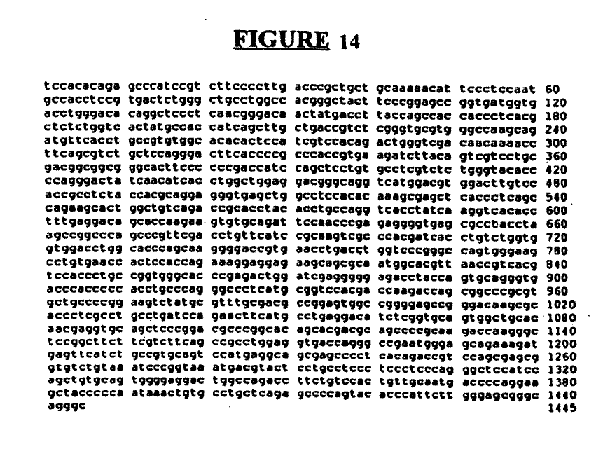

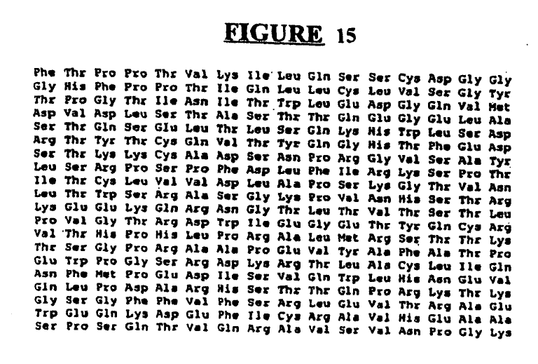

In another embodiment, the IgE fragment comprises an amino acid sequence having the CH2-CH3-CH4 domain amino acid sequence of the native IgE. Alternatively, the IgE fragment comprises at least part of the CH2, CH3 and CH4 domains of a native human IgE heavy chain constant region in the absence of a functional CH1 region. The IgE sequence includes variants of the IgE sequence which retain the biological activity of the IgE, including but not limited to the ability to bind to a native FcεRI and/or FcεRII receptor. In one embodiment the amino acid sequence of the constant region of the IgE is the sequence in FIG. 13 (SEQ ID NO:1).

The term “peptide”, “polypeptide”, or “protein” in singular or plural, is used herein to refer to any peptide or protein comprising two or more amino acids joined to each other in a linear chain by peptide bonds. As used herein, the term refers to both short chains, which also commonly are referred to in the art as peptides, oligopeptides and oligomers, and to longer chains, commonly referred to in the art as proteins. Polypeptides, as defined herein, may contain amino acids other than the 20 naturally occurring amino acids, and may include modified amino acids. The modification can be anywhere within the polypeptide molecule, such as, for example, at the terminal amino acids, and may be due to natural processes, such as processing and other post-translational modifications, or may result from chemical and/or enzymatic modification techniques which are well known to the art. The known modifications include, without limitation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature, such as, for instance, Creighton, T. E., Proteins—Structure And Molecular Properties, 2nd Ed., W. H. Freeman and Company, New York (1993); Wold, F., “Posttranslational Protein Modifications: Perspectives and Prospects,” in Posttranslational Covalent Modification of Proteins, Johnson, B. C., ed., Academic Press, New York (1983), pp. 1-12; Seifter et al., Meth. Enzymol. 182:626-646 (1990), and Rattan et al., Ann. N.Y Acad. Sci. 663:48-62 (1992).

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine. Accordingly, when glycosylation is desired, a polypeptide is expressed in a glycosylating host, generally eukaryotic host cells. Insect cells often carry out the same post-translational glycosylations as mammalian cells and, for this reason, insect cell expression systems have been developed to express efficiently mammalian proteins having native patterns of glycosylation.

It will be appreciated that polypeptides are not always entirely linear. For instance, polypeptides may be branched as a result of ubiquitination, and they may be circular, with or without branching, generally as a result of post-translational events, including natural processing and events brought about by human manipulation which do not occur naturally. Circular, branched and branched circular polypeptides may be synthesized by non-translation natural process and by entirely synthetic methods, as well. Such structures are within the scope of the polypeptides as defined herein.

Amino acids are represented by their common one- or three-letter codes, as is common practice in the art. Accordingly, the designations of the twenty naturally occurring amino acids are as follows: Alanine=Ala (A); Arginine=Arg (R); Aspartic Acid=Asp (D); Asparagine=Asn (N); Cysteine=Cys (C); Glutamic Acid=Glu (E); Glutamine=Gln (O); Glycine=Gly (G); Histidine=His (H); Isoleucine=Ile (I); Leucine=Leu (L); Lysine=Lys (K); Methionine=Met (M); Phenylalanine=Phe (F); Proline—Pro (P); Serine=Ser (S); Threonine=Thr (T); Tryptophan=Trp (W); Tyrosine=Tyr (Y); Valine=Val (V). The polypeptides herein may include all L-amino acids, all D-amino acids or a mixture thereof. The polypeptides comprised entirely of D-amino acids may be advantageous in that they are expected to be resistant to proteases naturally found within the human body, and may have longer half-lives.

The term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have at least one amino acid deleted in a particular region of the molecule.

The terms “fragment,” “portion” and “part,” as used interchangeably herein, refer to any composition of matter that is smaller than the whole of the composition of matter from which it is derived. For example, a portion of a polypeptide may range in size from two amino acid residues to the entire amino acid sequence minus one amino acid. However, in most cases, it is desirable for a “portion” or “fragment” to retain an activity or quality which is essential for its intended use. For example, useful portions of an antigen are those portions that retain an epitope determinant

The term “at least a portion,” as used herein, is intended to encompass portions as well as the whole of the composition of matter.

“Sequence identity” is defined as the percentage of amino acid residues in a candidate sequence that are identical with the amino acid residues in a reference polypeptide sequence (e.g., a native polypeptide sequence), after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1.

The term “sequence similarity” as used herein, is the measure of nucleic acid sequence identity, as described above, and in addition also incorporates conservative amino acid substitutions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions see Ausubel et al., Current Protocols in Molecular Biology, Wiley Interscience Publishers 1995).

“Stringent” hybridization conditions are sequence dependent and will be different with different environmental parameters (e.g., salt concentrations, and presence of organics). Generally, stringent conditions are selected to be about 5° C. to 20° C. lower than the thermal melting point (Tₘ) for the specific nucleic acid sequence at a defined ionic strength and pH. Stringent conditions are about 5° C. to 10° C. lower than the thermal melting point for a specific nucleic acid bound to a perfectly complementary nucleic acid. The Tₘ is the temperature (under defined ionic strength and pH) at which 50% of a nucleic acid (e.g., tag nucleic acid) hybridizes to a perfectly matched probe. “Stringent” wash conditions are ordinarily determined empirically for hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal to noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 400 mM, typically less than about 300 mM, less than about 200 mM, or less than about 150 mM. However, the combination of parameters is more important than the measure of any single parameter. See, e.g., Wetmur et al, J. Mol. Biol. 31:349-70 (1966), and Wetmur, Critical Reviews in Biochemistry and Molecular Biology 26(34):227-59 (1991).

In one embodiment, “stringent conditions” or “high stringency conditions,” as defined herein, may be hybridization in 50% formamide, 6×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (100 μg/ml), 0.5% SDS, and 10% dextran sulfate at 42° C., with washes at 42° C. in 2×SSC (sodium chloride/sodium citrate) and 0.1% SDS at 55° C., followed by a high-stringency wash consisting of 0.2×SSC containing 0.1% SDS at 42° C.

The terms “complement,” “complementarity” or “complementary,” as used herein, are used to describe single-stranded polynucleotides related by the rules of antiparallel base-pairing. For example, the sequence 5′-CTAGT-3′ is completely complementary to the sequence 5′-ACTAG-3′. Complementarity may be “partial,” where the base pairing is less than 100%, or complementarity may be “complete” or “total,” implying perfect 100% antiparallel complementation between the two polynucleotides. By convention in the art, single-stranded nucleic acid molecules are written with their 5′ ends to the left, and their 3′ ends to the right.

The term “DNA vaccine” means a DNA sequence which encodes a peptide. Upon entry into a mammalian cell, the DNA sequence will be translated into the peptide. It is contemplated that the DNA vaccine may comprise a DNA which encodes a fragment or portion of an allergen, a fragment or portion of an autoantigen or a fragment or portion of a virus.

The term “virus” refers to an infectious agent which infects mammalian cells. Examples of viruses include but are not limited to the HIV virus, herpes viruses, papillomavirus, hepatitis virus, varicellovirus, cytomegalovirus, paramyxovirus, mumps virus, rubella virus, pneumonia virus, rhinovirus etc.

The term “allergen,” and grammatical variants thereof, are used to refer to special antigens that are capable of inducing IgE-mediated allergies. An allergen can be almost anything that acts as an antigen and stimulates an IgE-mediated allergic reaction. Common allergens can be found, for example, in food, pollen, mold, house dust which may contain mites as well as dander from house pets, venom from insects such as bees, wasps and mosquitoes. Common allergens are listed in Table 1. In one embodiment the allergen is Fel d1. In another embodiment that allergen is the peanut allergen Ara h or Arachis hypogea or egg allergen ovomucoid (Gal d1) or milk allergen acasein.

The term “antigen,” as used herein, refers to any agent that is recognized by an antibody, while the term “immunogen” refers to any agent that can elicit an immunological response in a subject. The terms “antigen” and “immunogen” both encompass, but are not limited to, polypeptides. In most, but not all cases, antigens are also immunogens.

The terms “autoantigen” and “self antigen” and grammatical equivalents, as used herein, refer to an antigen endogenous to an individual's physiology, that is recognized by either the cellular component (T-cell receptors) or humoral component (antibodies) of that individual's immune system. The presence of autoantigens, and consequently autoantibodies and/or self-reactive T-cells, is frequently, but not absolutely, associated with disease states. Autoantibodies may be detected in disease-free individuals. Autoantigens are frequently, but not exclusively, polypeptides. An understanding of the mechanisms underlying the recognition of autoantigens, the loss of normal self-recognition, or the mechanisms inducing autoimmunity are not necessary to make or use the present invention.

The term “autoantibody,” as used herein, is intended to refer to any antibody produced by a host organism that binds specifically to an autoantigen, as defined above. The presence of autoantibodies and/or self-reactive T-cells is referred to herein as “autoimmunity.” The presence of autoantibodies or self-reactive T-cells in a subject is frequently, but not absolutely, associated with disease (i.e., autoimmune disease).

The terms “epitope” or “antigenic determinant” as used herein, refer to that portion of an antigen that forms the region that reactions with a particular antibody variable region, and thus imparts specificity to the antigen/antibody binding. A single antigen may have more than one epitope. An immunodominant epitope is an epitope on an antigen that is preferentially recognized by antibodies to the antigen. In some cases, where the antigen is a protein, the epitope can be “mapped,” and an “antigenic peptide” produced corresponding approximately to just those amino acids in the protein that are responsible for the antibody/antigen specificity. Such “antigenic peptides” find use in peptide immunotherapies.

The terms “autoimmune disease,” “autoimmune condition” or “autoimmune disorder,” as used interchangeably herein, refer to a set of sustained organ-specific or systemic clinical symptoms and signs associated with altered immune homeostasis that is manifested by qualitative and/or quantitative defects of expressed autoimmune repertoires. Autoimmune disease pathology is manifested as a result of either structural or functional damage induced by the autoimmune response. Autoimmune diseases are characterized by humoral (e.g., antibody-mediated), cellular (e.g., cytotoxic T lymphocyte-mediated), or a combination of both types of immune responses to epitopes on self-antigens. The immune system of the affected individual activates inflammatory cascades aimed at cells and tissues presenting those specific self-antigens. The destruction of the antigen, tissue, cell type or organ attacked gives rise to the symptoms of the disease. The autoantigens are known for some, but not all, autoimmune diseases.

The terms “immunotherapy,” “desensitisation therapy,” “hyposensitisation therapy,” “tolerance therapy” and the like, as used herein, describe methods for the treatment of various hypersensitivity disorders, where the avoidance of an allergen or autoantigen is not possible or is impractical. As used herein, these terms are used largely interchangeably. These methods generally entail the delivery to a subject of the antigenic material in a controlled manner to induce tolerance to the antigen and/or downregulate an immune response that occurs upon environmental exposure to the antigen. These therapies typically entail injections of the antigen (e.g., an allergen or autoantigen) over an extended period of time (months or years) in gradually increasing doses. The antigen used in the immunotherapies is typically, but not exclusively, polypeptides. For example, hay fever desensitisation therapy downregulates allergic response to airborn pollen, where the subject is injected with a pollen extract. From a clinical perspective, these treatments are suboptimal, as the injections are often uncomfortable, as well as inconvenient. Furthermore, a significant risk of potentially life-threatening anaphylactic responses during the therapies exists. Adapting immunotherapy techniques for the treatment of various autoimmune disorders has been proposed, where the autoantigen is administered to a subject in the hope of inducing tolerance to the autoantigen, and thereby eliminating the immune destruction of the endogenous autoantigen or autoantigenic tissue. For example, insulin and myelin-basic-protein have been delivered to animal models and humans for the purpose of downregulating autoimmune type-I diabetes mellitus and multiple sclerosis, respectively.

The terms “peptide therapy” and “peptide immunotherapy,” and the like, as used herein, describe methods of immunotherapy, wherein the antigen (e.g., an allergen or autoantigen) delivered to a subject is a short polypeptide (i.e., a peptide). Furthermore, the peptide delivered during peptide therapy may contain only those amino acids defining an immunodominant epitope (e.g., the myelin-basic-protein epitope (MBP).

The terms “vaccine therapy,” “vaccination” and “vaccination therapy,” as used interchangeably herein, refer in general to any method resulting in immunological prophylaxis. In one aspect, vaccine therapy induces an immune response, and thus long-acting immunity, to a specific antigen. These methods generally entail the delivery to a subject of an immunogenic material to induce immunity. In another aspect, the “vaccine therapy” refers to a method for the downregulation of an immune potential to a particular antigen (e.g., to suppress an allergic response). This type of vaccine therapy is also referred to as “tolerance therapy.”

A “Type I” allergic reaction or “immediate hypersensitivity” or “atopic allergy” occurs when an antigen entering the body encounters mast cells or basophils that have been sensitized by IgE attached to its high-affinity receptor, FcεRI on these cells. When an allergen reaches the sensitized mast cell or basophil, it cross-links surface-bound IgE, causing an increase in intracellular calcium (Ca²⁺) that triggers the release of pre-formed mediators, such as histamine and proteases, and newly synthesized, lipid-derived mediators such as leukotrienes and prostaglandins. These autocoids produce the clinical symptoms of allergy. In addition, cytokines, e.g. IL-4, TNF-alpha, are released from degranulating basophils and mast cells, and serve to augment the inflammatory response that accompanies an IgE reaction (see, e.g. Immunology, Fifth Edition, Roitt et al., eds., 1998, pp. 302-317).

Symptoms and signs associated with type I hypersensitivity responses are extremely varied due to the wide range of tissues and organs that can be involved. These symptoms and signs can include, but are not limited to: itching of the skin, eyes, and throat, swelling and rashes of the skin (angioedema and urticaria/hives), hoarseness and difficulty breathing due to swelling of the vocal cord area, a persistent bumpy red rash that may occur anywhere on the body, shortness of breath and wheezing (from tightening of the muscles in the airways and plugging of the airways, i.e., bronchoconstriction) in addition to increased mucus and fluid production, chest tightness and pain due to construction of the airway muscles, nausea, vomiting diarrhea, dizziness and fainting from low blood pressure, a rapid or irregular heartbeat and even death as a result of airway and/or cardiac compromise.

Examples of disease states that result from allergic reactions, and demonstrating hypersensitivity symptoms and/or signs include, but are not limited to, allergic rhinitis, allergic conjunctivitis, atopic dermatitis, allergic [extrinsic] asthma, some cases of urticaria and angioedema, food allergy, and anaphylactic shock in which there is systemic generalized reactivity and loss of blood pressure that may be fatal.

The terms “anaphylaxis,” “anaphylactic response,” “anaphylactic reaction,” “anaphylactic shock,” and the like, as used interchangeably herein, describe the acute, often explosive, IgE-mediated systemic physiological reaction that occurs in a previously sensitized subject who receives the sensitizing antigen. Anaphylaxis occurs when the previously sensitizing antigen reaches the circulation. When the antigen reacts with IgE on basophils and mast cells, histamine, leukotrienes, and other inflammatory mediators are released. These mediators cause the smooth muscle contraction (responsible for wheezing and gastrointestinal symptoms) and vascular dilation (responsible for the low blood pressure) that characterize anaphylaxis. Vasodilation and escape of plasma into the tissues causes urticaria and angioedema and results in a decrease in effective plasma volume, which is the major cause of shock. Fluid escapes into the lung alveoli and may produce pulmonary edema. Obstructive angioedema of the upper airway may also occur. Arrhythmias and cardiogenic shock may develop if the reaction is prolonged. The term “anaphylactoid reaction” refers to a physiological response that displays characteristics of an anaphylactic response.

Symptoms of an anaphylactic reaction vary considerably among patients. Typically, in about 1 to 15 minutes (but rarely after as long as 2 hours), symptoms can include agitation and flushing, palpitations, paresthesias, pruritus, throbbing in the ears, coughing, sneezing, urticaria and angioedema, vasodilation, and difficulty breathing owing to laryngeal edema or bronchospasm. Nausea, vomiting, abdominal pain, and diarrhea are also sometimes observed. Shock may develop within another 1 or 2 minutes, and the patient may convulse, become incontinent, unresponsive, and succumb to cardiac arrest, massive angioedema, hypovolemia, severe hypotension and vasomotor collapse and primary cardiovascular collapse. Death may ensue at this point if the antagonist epinephrine is not immediately available. Mild forms of anaphylactic response result in various symptoms including generalized pruritus, urticaria, angioedema, mild wheezing, nausea and vomiting. Patients with the greatest risk of anaphylaxis are those who have reacted previously to a particular drug or antigen.

The term “nucleic acid binding agent” means an agent which binds to the nucleic acid. Such agents include a retroviral coat, an adenovirus coat, another viral or viral-like form (such as herpes simplex, and adeno-associated virus (AAV) coat), liposomes, poly-lysine, Poly-l-lysine (PLL), poly-arginine-lysine, poly-l-arginine-lysine (PRL), synthetic polycationic molecules, polyethylene glycol (PEG), spermine or spermidine.

The terms “vector”, “polynucleotide vector”, “construct” and “polynucleotide construct” are used interchangeably herein. A polynucleotide vector of this invention may be in any of several forms, including, but not limited to, RNA, DNA. In one embodiment the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “host cell” includes an individual cell or cell culture which can be or has been a recipient of any vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a nucleic acid of the present invention.

The term “promoter” means a nucleotide sequence that, when operably linked to a DNA sequence of interest, promotes transcription of that DNA sequence. It is contemplated that the promoter will be a “dendritic cell promoter” which means that the promoter is active in dendritic cells. It is further contemplated that the “dendritic cell promoter will have reduced activity or no activity in other cells expressing the IgE receptors. It is contemplated the “dendritic cell promoter” will be the Fascin promoter (Sudowe, S., et al., 2006. “Prophylactic and therapeutic intervention in IgE responses by biolistic DNA vaccination primarily targeting dendritic cells”. J Allergy Clin Immunol. 117:196-203),

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The term “IgE-mediated biological response” is used to refer to a condition or disease which is characterized by signal transduction through an IgE receptor, including the high-affinity IgE receptor, FcεRII, and the low-affinity IgE receptor FcεRII. The definition includes, without limitation, conditions associated with anaphylactic hypersensitivity and atopic allergies, such as, for example, asthma, allergic rhinitis, atopic dermatitis, food allergies, chronic urticaria and angioedema, as well as the serious physiological condition of anaphylactic shock, usually caused by bee stings or medications such as penicillin.

The terms “treat” or “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain a desired effect or level of agent(s) for an extended period of time.

“Intermittent” administration is treatment that is not consecutively done without interruption, but rather is periodic in nature.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

“Carriers” or “pharmaceutically acceptable ingredients” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “mammal” or “mammalian species” refers to any animal classified as a mammal, including humans, domestic and farm animals, and zoo, sports, or pet animals, such as dogs, cats, cattle, horses, sheep, pigs, goats, rabbits, as well as rodents such as mice and rats, etc. In one embodiment the mammal is human.

The terms “subject” or “patient,” as used herein, are used interchangeably, and can refer to any animal, and in one embodiment a mammal, that is the subject of an examination, treatment, analysis, test or diagnosis. In one embodiment, humans are the subject. A subject or patient may or may not have a disease or other pathological condition.

The terms “disease,” “disorder” and “condition” are used interchangeably herein, and refer to any disruption of normal body function, or the appearance of any type of pathology. The etiological agent causing the disruption of normal physiology may or may not be known. Furthermore, although two patients may be diagnosed with the same disorder, the particular symptoms displayed by those individuals may or may not be identical.

### II. DETAILED DESCRIPTION

The invention concerns an novel approach for focusing and expressing DNA vaccines in Antigen Presenting Cells (APCs) mediated through targeting IgE receptors (FcεRs) on APC and driving DNA expression through provision of an APC specific regulatory element. Such improved DNA vaccines will be useful in the management of IgE-mediated allergic diseases and other disorders, e.g. autoimmune disorders, infectious diseases such as viral diseases and cancer were DNA vaccination may have a beneficial effect.

A large proportion of asthma cases, and particularly those seen in infants, children and young adults, are related to allergic responses to environmental allergens. This is directed toward the development of a new form of gene transfer intervention designed to lead to induction of long-term remission (allergic tolerance) in human allergic asthma. The vaccine comprises a human IgE and a highly human relevant allergen, cat Fel d1. Cat allergen is a major allergen for humans and because of its size and highly buoyant nature, it is widely distributed, including being found in public buildings such as schools. Indeed, nearly half of all homes without a cat in residence have enough cat allergen present to potentially cause symptoms in cat allergic subjects (Arbes S J Jr, Cohn R D, Yin M, Muilenberg M L, Friedman W, Zeldin D C. (2004). J Allergy Clin Immunol. 114:111). While various new and important drug treatments have been developed for the short and long term treatment of asthma, (e.g. better topical steroids, leukotriene inhibitors and anti-IgE), treatment of asthma remains problematic and there continues to be a worldwide an epidemic of increased asthma incidence and severity. New treatments aimed at long-term disease remission such as we are proposing deserve to be aggressively investigated.

Allergen gene vaccination represents a promising alternative to the protein-based immunotherapy approach for allergen-specific immunotherapy to treat allergic asthma and other allergic conditions. This approach has been shown to effectively inhibit allergen-specific IgE production, suppress Th2 response, and reciprocally enhance Th1 response. However, the development of allergen DNA vaccination has been limited by the inefficiency of the gene delivery methods for the delivery of the DNA to professional antigen presenting cells (APCs) and particularly dendritic cells (DCs). We will take advantage of the fact that human APCs, particularly DCs and Langerhans cells (LC), express high affinity receptors for IgE (FcεRI) and thereby focus the gene of interest on these cells by constructing and administering a combined allergen encoding DNA-human IgE molecular complex called a “polyplex”. This polyplex will be delivered to APCs through the IgE-FcεRI interaction, which has an extraordinarily high affinity with a Kd between 10⁻¹⁰ to 10⁻¹¹ L/M, an affinity 2 to 3 logs higher than most physiological ligand-receptor (antigen-antibody) interactions. This novel approach will provide a highly efficient IgE-mediated DNA vaccine delivery to FcεRI expressing APCs for allergen immunotherapy.

This high affinity IgE-FcεRI interaction can be utilized to facilitate the allergen gene vaccination by specifically targeting the gene of interest to human FcεRI (h FcεRI) expressing DCs and LCs. Mice carrying a transgene (Tg) for the human FcεRIα chain that model the high level of FcεRI expression by APCs of allergic patients will be used as the model system target for effective allergen DNA vaccination so as to modulate allergen-specific responses and treat allergic diseases, including allergic asthma. Expression of the allergen gene in targeted FcεRI expressing DCs rather than other FcεRI bearing cells, e.g. mast cells and basophils, will be accomplished by employing the actin-bundling protein fascin gene promoter in the construct, as it is specifically activated in DCs and not other FcεRI cells. Due to the very high affinity of the IgE-FcεRI interaction and the predicted efficiency of the IgE-mediated allergen gene transfer, the dose and frequency of DNA vaccinations required for efficient immunotherapy using our approach should be significantly lower compared to that of other immunization protocols and potential side effects/toxicity are likewise expected to be fewer.

It is required that IgE fragment retain the ability to bind to the corresponding native receptor, such as a native high-affinity IgE receptor (e.g. FcεRI) or native low-affinity IgE receptor (FcεRII, CD23). The receptor binding domains within the native IgE heavy chain constant region sequences have been identified. Based on FcεRI binding studies, Presta et al., J. Biol. Chem. 269:26368-26373 (1994) proposed that six amino acid residues (Arg-408, Ser-411, Lys-415, Glu-452, Arg-465, and Met-469) located in three loops, C-D, E-F, and F-G, computed to form the outer ridge on the most exposed side of the human IgE heavy chain CH3 domain, are involved in binding to the high-affinity receptor FcεRI, mostly by electrostatic interactions. Helm et al., J. Cell Biol. 271(13):7494-7500 (1996), reported that the high-affinity receptor binding site in the IgE molecule includes the Pro343-Ser353 peptide sequence within the CH3 domain of the IgE heavy chain, but sequences N— or C-terminal to this core peptide are also necessary to provide structural scaffolding for the maintenance of a receptor binding conformation. In particular, they found that residues, including His, in the C-terminal region of the ε-chain make an important contribution toward the maintenance of the high-affinity of interaction between IgE and FcεRII. The FCE polypeptide sequence are designed to bind to residues within such binding regions.

Based on this knowledge, the amino acid sequence variants may be designed to retain the native amino acid residues essential for receptor binding, or to perform only conservative amino acid alterations (e.g. substitutions) at such residues.

In making amino acid sequence variants that retain the required binding properties of the corresponding native sequences, the hydropathic index of amino acids may be considered. For example, it is known that certain amino acids may be substituted for other amino acids having a similar hydropathic index or score without significant change in biological activity. Thus, isoleucine, which has a hydrophatic index of +4.5, can generally be substituted for valine (+4.2) or leucine (+3.8), without significant impact on the biological activity of the polypeptide in which the substitution is made. Similarly, usually lysine (−3.9) can be substituted for arginine (−4.5), without the expectation of any significant change in the biological properties of the underlying polypeptide.

Other considerations for choosing amino acid substitutions include the similarity of the side-chain substituents, for example, size, electrophilic character, charge in various amino acids. In general, alanine, glycine and serine; arginine and lysine; glutamate and aspartate; serine and threonine; and valine, leucine and isoleucine are interchangeable, without the expectation of any significant change in biological properties. Such substitutions are generally referred to as conservative amino acid substitutions, and, as noted above, are one type of substitutions within the polypeptides of the present invention.

Alternatively or in addition, the amino acid alterations may serve to enhance the receptor binding properties of the IgE molecules of the invention. Variants with improved receptor binding and, as a result, superior biological properties can be readily designed using standard mutagenesis techniques, such as alanine-scanning mutagenesis, PCR mutagenesis or other mutagenesis techniques, coupled with receptor binding assays, such as the assay discussed below or described in the Example.

Receptor binding can be tested using any known assay method, such as competitive binding assays, direct and indirect sandwich assays. Thus, the binding of IgE polypeptide included herein to a high-affinity or low-affinity IgE receptor can be tested using conventional binding assays, such as competitive binding assays, including RIAs and ELISAs. Ligand/receptor complexes can be identified using traditional separation methods as filtration, centrifugation, flow cytometry, and the results from the binding assays can be analyzed using any conventional graphical representation of the binding data, such as Scatchard analysis. The assays may be performed, for example, using a purified receptor, or intact cells expressing the receptor. One or both of the binding partners may be immobilized and/or labeled. A particular cell-based binding assay is described in the Example below.

The polyplex comprises a combined allergen encoding DNA attached to an IgE molecular complex. In one embodiment the IgE molecular complex comprises an IgE fragment attached to a nucleic acid binding agent. The nucleic acid binding agent may comprise an amino acid chain, for example, poly-lysine or polyarginine-lysine. In one embodiment the poly-lysine is poly-l-lysine (PLL). It is contemplated that the poly-l-lysine may contain at least about 10 lysine residues, at least about 20 lysine residues, at least about 30 lysine residues, at least about 60 lysine residues. In another embodiment the poly-arginine-lysine is poly-l-arginine lysine (PRL) comprising alternating residues of arginine and lysine. It is contemplated that the poly-l-arginine-lysine may contain at least about 10 amino acid residues, at least about 20 residues, at least about 30 residues, at least about 60 residues, at least about 80 residues. It is further contemplated that the arginine and lysine residues may not alternate but may be in a random order such as ARG-ARG-ARG-LYS-LYS-ARG etc.

In another embodiment, the IgE and nucleic acid binding agent may be connected by a polypeptide linker. The polypeptide linker functions as a “spacer”. The polypeptide linker usually comprises between about 1 and about 25 residues or from about 2 to about 25 residues. The polypeptide linker may contain at least about 10, or at least about 15 amino acids. The polypeptide linker may be composed of amino acid residues which together provide a hydrophilic, relatively unstructured region. Linking amino acid sequences with little or no secondary structure work well. The specific amino acids in the spacer can vary, however, cysteines should be avoided. Suitable polypeptide linkers are, for example, disclosed in WO 88/09344 (published on Dec. 1, 1988), as are methods for the production of multifunctional proteins comprising such linkers.

It is contemplated that the polyplex of the IgE fragment and the nucleic acid binding agent may further include a cellular uptake sequence. Such a cellular uptake sequence would enhance the cellular uptake of the polyplex and the expression of the allergen vaccine. In one embodiment that cellular uptake sequence may be the HIV tat peptide sequence and/or a nuclear localization signal (NLS) peptide sequence. The cellular uptake sequence may be placed between the IgE fragment and the PLL peptide sequence. The HIV tat peptide sequence may be GRKKRRQRRR (SEQ ID NO:4). The NLS peptide sequence may be PKKKRKV (SEQ ID NO: 5).

It is contemplated that a recombinant DNA technique may be used to generate the IgE sequence and the nucleic acid binding agent amino acid sequence. A fusion gene comprising the DNA sequence for the human IgE heavy chain (CHε2-CHε3-CHε4) linked with DNA encoding the nucleic acid binding agent amino acid sequence is generated. This approach would ensure that each IgE molecule is associated with nucleic acid binding agent, and the quality of the product would be the same for all the experiments performed at different times. In one embodiment, the nucleic acid binding agent would be encoded by 180 by DNA coding for 60 repeated lysines.

The IgE sequence and the nucleic acid binding agent may be connected by a non-polypeptide linker. Such linkers may, for example, be residues of covalent bifunctional cross-linking agents capable of linking the two sequences without the impairment of the receptor (antibody) binding function. The bifunctional cross-linking reagents can be divided according to the specificity of their functional groups, e.g. amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group (for review, see Ji, T. H. “Bifunctional Reagents” in: Meth. Enzymol. 91:580-609 (1983)).

The vaccine of the present invention can be used to inhibit FcεR mediated biological responses. Such biological responses are the mediation of an allergic reactions or autoimmune reactions via FcεR, including, without limitation, conditions associated with IgE mediated reactions, such as, for example, asthma, allergic rhinitis, food allergies, chronic urticaria and angioedema, allergic reactions to hymenophthera (e.g. bee and yellow jacket) stings or medications such as penicillin up to and including the severe physiological reaction of anaphylactic shock.

In one embodiment, the allergen DNA sequence encodes allergens selected from the allergen sequences listed in Table 1 below.

TABLE 1

| | | SWISS- | | |
|---|---|---|---|---|
| | | PROT | | |
| | SWISS-PROT | Accession | | |
| Allergen | Entry | No. | Protein Name | Source |
| Aln g 1 | MPAG_ALNGL | P38948 | Major Pollen Allergen | Pollen of Alnus |
| | | | Aln g 1 | glutinosa (Alder) |
| Alt a 6 | RLA2_ALTAL | P42037 | 60S Acidic Ribosomal | Alternaria alternata |
| | | | Protein P2 | |
| Alt a 7 | ALA7_ALTAL | P42058 | Minor Allergen Alt a 7 | Alternaria alternata |
| Alt a 10 | DHAL_ALTAL | P42041 | Aldehyde | Alternaria alternata |
| | | | Dehydrogenase | |
| Alt a 12 | RLA1_ALTAL | P49148 | 60S Acidic Ribosomal | Alternaria alternata |
| | | | Protein P1 | |
| Amb a 1 | MP11_AMBAR | P27759 | Pollen Allergen Amb | Ambrosia |
| | | | a 1.1 [Precursor] | artemisiifolia (Short |
| | | | | ragweed) |
| Amb a 1 | MP12_AMBAR | P27760 | Pollen Allergen Amb | Ambrosia |
| | | | a 1.2 [Precursor] | artemisiifolia (Short |
| | | | | ragweed) |
| Amb a 1 | MP13_AMBAR | P27761 | Pollen Allergen Amb | Ambrosia |
| | | | a 1.3 [Precursor] | artemisiifolia (Short |
| | | | | ragweed) |
| Amb a 1 | MP14_AMBAR | P28744 | Pollen Allergen Amb | Ambrosia |
| | | | a 1.4 [Precursor] | artemisiifolia (Short |
| | | | | ragweed) |
| Amb a 2 | MPA2_AMBAR | P27762 | Pollen Allergen Amb | Ambrosia |
| | | | a 2 [Precursor] | artemisiifolia (Short |
| | | | | ragweed) |
| Amb a 3 | MPA3_AMBEL | P00304 | Pollen Allergen Amb | Ambrosia |
| | | | a 3 | artemisiifolia var. |
| | | | | elatior (Short |
| | | | | ragweed) |
| Amb a 5 | MPA5_AMBEL | P02878 | Pollen Allergen Amb | Ambrosia |
| | | | a 5 | artemisiifolia var. |
| | | | | elatior (Short |
| | | | | ragweed) |
| Amb p 5 | MPA5_AMBPS | P43174 | Pollen Allergen Amb | Ambrosia |
| | | | p 5-a [Precursor] | psilostachya (Western |
| | | | | ragweed) |
| Amb p 5 | MP5B_AMBPS | P43175 | Pollen Allergen Amb | Ambrosia |
| | | | p 5b [Precursor] | psilostachya (Western |
| | | | | ragweed) |
| Amb t 5 | MPT5_AMBTR | P10414 | Pollen Allergen Amb | Ambrosia trifida |
| | | | t 5 [Precursor] | (Giant ragweed) |
| Api g 1 | MPAG_APIGR | P49372 | Major Allergen Api g 1 | Apium grayeolens |
| | | | | (Celery) |
| Api m 1 | PA2_APIME | P00630 | Phospholipase A2 | Apis mellifera |
| | | | [Precursor] | (Honeybee) |
| | | | [Fragment] | |
| Api m 2 | HUGA_APIME | Q08169 | Hyaluronoglucosaminidase | Apis mellifera |
| | | | [Precursor] | (Honeybee) |
| Api m 3 | MEL_APIME | P01501 | Melittin [Precursor] | Apis mellifera |
| | | | | (Honeybee) Apis |
| | | | | cerana (Indian |
| | | | | honeybee) |
| Ara h 1 | AH11_ARAHY | P43237 | Allergen Ara h 1, | Arachis hypogaea |
| | | | Clone P17 | (Peanut) |
| Ara h 1 | AH12_ARAHY | P43238 | Allergen Ara h 1, | Arachis hypogaea |
| | | | Clone P41b | (Peanut) |
| Ara t 8 | PRO1_ARATH | Q42449 | Profilin 1 | Arabidopsis thaliana |
| | | | | (Mouse-ear cress) |
| Asp f 1 | RNMG_ASPRE | P04389 | Ribonuclease | Aspergillus restrictus; |
| | | | Mitogillin [Precursor] | Aspergillus fumigatus |
| | | | | (Sartorya fumigata) |
| Asp f 2 | MAF2_ASPFU | P79017 | Major Allergen Asp f | Aspergillus fumigatus |
| | | | 2 [Precursor] | (Sartorya fumigata) |
| Asp f 3 | PM20_ASPFU | O43099 | Probable Peroxisomal | Aspergillus fumigatus |
| | | | Membrane Protein | (Sartorya fumigata) |
| | | | PMP20 | |
| Asp f 13 | AF13_ASPFU | O60022 | Allergen Asp f | Aspergillus fumigatus |
| | | | 13 [Precursor] | (Sartorya fumigata) |
| Bet v 1 | BV1A_BETVE | P15494 | Major Pollen Allergen | Betula verrucosa |
| | | | Bet v 1-a | (White birch) (Betula |
| | | | | pendula) |
| Bet v 1 | BV1C_BETVE | P43176 | Major Pollen Allergen | Betula verrucosa |
| | | | Bet v 1-c | (White birch) (Betula |
| | | | | pendula) |
| Bet v 1 | BV1D_BETVE | P43177 | Major Pollen Allergen | Betula verrucosa |
| | | | Bet v 1-d/h | (White birch) (Betula |
| | | | | pendula) |
| Bet v 1 | BV1E_BETVE | P43178 | Major Pollen Allergen | Betula verrucosa |
| | | | Bet v 1-e | (White birch) (Betula |
| | | | | pendula) |
| Bet v 1 | BV1F_BETVE | P43179 | Major Pollen Allergen | Betula verrucosa |
| | | | Bet v 1-f/i | (White birch) (Betula |
| | | | | pendula) |
| Bet v 1 | BV1G_BETVE | P43180 | Major Pollen Allergen | Betula verrucosa |
| | | | Bet v 1-g | (White birch) (Betula |
| | | | | pendula) |
| Bet v 1 | BV1J_BETVE | P43183 | Major Pollen Allergen | Betula verrucosa |
| | | | Bet v 1-j | (White birch) (Betula |
| | | | | pendula) |
| Bet v 1 | BV1K_BETVE | P43184 | Major Pollen Allergen | Betula verrucosa |
| | | | Bet v 1-k | (White birch) (Betula |
| | | | | pendula) |
| Bet v 1 | BV1L_BETVE | P43185 | Major Pollen Allergen | Betula verrucosa |
| | | | Bet v 1-l | (White birch) (Betula |
| | | | | pendula) |
| Bet v 1 | BV1M_BETVE | P43186 | Major Pollen Allergen | Betula verrucosa |
| | | | Bet v 1-m/n | (White birch) (Betula |
| | | | | pendula) |
| Bet v 2 | PROF-BETVE | P25816 | Profilin | Betula verrucosa |
| | | | | (White birch) (Betula |
| | | | | pendula) |
| Bet v 3 | BTV3_BETVE | P43187 | Allergen Bet v 3 | Betula verrucosa |
| | | | | (White birch) (Betula |
| | | | | pendula) |
| Bla g 2 | ASP2_BLAGE | P54958 | Aspartic Protease Bla | Blattella germanica |
| | | | g 2 [Precursor] | (German cockroach) |
| Bla g 4 | BLG4_BLAGE | P54962 | Allergen Bla g 4 | Blattella germanica |
| | | | [Precursor] | (German cockroach) |
| | | | [Fragment] | |
| Bla g 5 | GTS1_BLAGE | O18598 | Glutathione-S- | Blattella germanica |
| | | | transferase | (German cockroach) |
| Blo t 12 | BT12_BLOTA | Q17282 | Allergen Blo t 12 | Blomia tropicalis |
| | | | [Precursor] | (Mite) |
| Bos d 2 | ALL2_BOVIN | Q28133 | Allergen Bos d 2 | Bos taurus (Bovine) |
| | | | [Precursor] | |
| Bos d 5 | LACB_BOVIN | P02754 | Beta-lactoglobulin | Bos taurus (Bovine) |
| | | | [Precursor] | |
| Bra j 1 | ALL1_BRAJU | P80207 | Allergen Bra j 1-e, | Brassica juncea (Leaf |
| | | | Small and Large | mustard) (Indian |
| | | | Chains | mustard) |
| Can a 1 | ADH1_CANAL | P43067 | Alcohol | Candida albicans |
| | | | Dehydrogenase 1 | (Yeast) |
| Can f 1 | ALL1_CANFA | O18873 | Major Allergen Can f | Canis famiiaris (Dog) |
| | | | 1 [Precursor] | |
| Can f 2 | ALL2_CANFA | O18874 | Minor Allergen Can f | Canis familiaris (Dog) |
| | | | 2 [Precursor] | |
| Car b 1 | MPA1_CARBE | P38949 | Major Pollen Allergen | Carpinus betulus |
| | | | Car b 1, Isoforms 1A | (Hornbeam) |
| | | | and 1B | |
| Car b 1 | MPA2_CARBE | P38950 | Major Pollen Allergen | Carpinus betulus |
| | | | Car b 1, Isoform 2 | (Hornbeam) |
| Cha o 1 | MPA1_CHAOB | Q96385 | Major Pollen Allergen | Chamaecyparis obtusa |
| | | | Cha o 1 [Precursor] | (Japanese cypress) |
| Cla h 3 | DHAL_CLAHE | P40108 | Aldehyde | Cladosporium |
| | | | Dehydrogenase | herbarum |
| Cla h 3 | RLA3_CLAHE | P42038 | 60S Acidic Ribosomal | Cladosporium |
| | | | Protein P2 | herbarum |
| Cla h 4 | HS70_CLAHE | P40918 | Heat Shock 70 KDa | Cladosporium |
| | | | Protein | herbarum |
| Cla h 4 | RLA4_CLAHE | P42039 | 60S Acidic Ribosomal | Cladosporium |
| | | | Protein P2 | herbarum |
| Cla h 5 | CLH5_CLAHE | P42059 | Minor Allergen Cla h 5 | Cladosporium |
| | | | | herbarum |
| Cla h 6 | ENO_CLAHE | P42040 | Enolase | Cladosporium |
| | | | | herbarum |
| Cla h 12 | RLA1_CLAHE | P50344 | 60S Acidic Ribosomal | Cladosporium |
| | | | Protein P1 | herbarum |
| Cop c 2 | THIO_CAPCM | | | |
| Cor a 1 | MPAA_CORAV | Q08407 | Major Pollen Allergen | Corylus avellana |
| | | | Cor a 1, Isoforms 5, 6, | (European hazel) |
| | | | 11 and 16 | |
| Cup a 1 | MPA1_CUPAR | Q9SCG9 | Major Pollen Allergen | Cupressus arizonica |
| | | | Cup a 1 | |
| Cry j 1 | SBP_CRYJA | P18632 | Sugi Basic Protein | Cryptomeria japonica |
| | | | [Precursor] | (Japanese cedar) |
| Cry j 2 | MPA2_CRYJA | P43212 | Possible | Cryptomeria japonica |
| | | | Polygalacturonase | (Japanese cedar) |
| Cyn d 12 | PROF_CYNDA | O04725 | Profilin | Cynodon dactylon |
| | | | | (Bermuda grass) |
| Dac g 2 | MPG2_DACGL | Q41183 | Pollen Allergen Dac g | Dactylis glomerata |
| | | | 2 [Fragment] | (Orchard grass) |
| | | | | (Cocksfoot grass) |
| Dau c 1 | DAU1_DAUCA | O04298 | Major Allergen Dau c 1 | Daucus carota |
| | | | | (Carrot) |
| Der f 1 | MMAL_DERFA | P16311 | Major Mite Fecal | Dermatophagoides |
| | | | Allergen Der f 1 | farinae (House-dust |
| | | | [Precursor] | mite) |
| Der f 2 | DEF2_DERFA | Q00855 | Mite Allergen Der f 2 | Dermatophagoides |
| | | | [Precursor] | ferinae (House-dust |
| | | | | mite) |
| Der f 3 | DEF3_DERFA | P49275 | Mite Allergen Der f 3 | Dermatophagoides |
| | | | [Precursor] | ferinae (House-dust |
| | | | | mite) |
| Der f 6 | DEF6_DERFA | P49276 | Mite Allergen Der f 6 | Dermatophagoides |
| | | | [Fragment] | ferinae (House-dust |
| | | | | mite) |
| Der f 7 | DEF7_DERFA | Q26456 | Mite Allergen Der f 7 | Dermatophagoides |
| | | | [Precursor] | ferinae (House-dust |
| | | | | mite) |
| Der m 1 | MMAL_DERMI | P16312 | Major Mite Fecal | Dermatophagoides |
| | | | Allergen Der m 1 | microceras (House- |
| | | | [Fragment] | dust mite) |
| Der p 1 | MMAL_DERPT | P08176 | Major Mite Fecal | Dermatophagoides |
| | | | Allergen Der p 1 | pteronyssinus (House- |
| | | | [Precursor] | dust mite) |
| Der p 2 | DER2_DERPT | P49278 | Mite Allergen Der p 2 | Dermatophagoides |
| | | | [Precursor] | pteronyssinus (House- |
| | | | | dust mite) |
| Der p 3 | DER3_DERPT | P39675 | Mite Allergen Der p 3 | Dermatophagoides |
| | | | [Precursor] | pteronyssinus (House- |
| | | | | dust mite) |
| Der p 4 | AMY_DERPT | P49274 | Alpha-Amylase | Dermatophagoides |
| | | | [Fragment] | pteronyssinus (House- |
| | | | | dust mite) |
| Der p 5 | DER5_DERPT | P14004 | Mite Allergen Der p 5 | Dermatophagoides |
| | | | | pteronyssinus (House- |
| | | | | dust mite) |
| Der p 6 | DER6_DERPT | P49277 | Mite Allergen Der p 6 | Dermatophagoides |
| | | | [Fragment] | pteronyssinus (House- |
| | | | | dust mite) |
| Der p 7 | DER7_DERPT | P49273 | Mite Allergen Der p 7 | Dermatophagoides |
| | | | [Precursor] | pteronyssinus (House- |
| | | | | dust mite) |
| Dol a 5 | VA5_DOLAR | Q05108 | Venom Allergen 5 | Dolichovespula |
| | | | | arenaria (Yellow |
| | | | | hornet) |
| Dol m 1 | PA11_DOLMA | Q06478 | Phospholipase A1 1 | Dolichovespula |
| | | | [Precursor] | maculata (White-face |
| | | | [Fragment] | hornet) (Bald-faced |
| | | | | hornet) |
| Dol m 1 | PA12_DOLMA | P53357 | Phospholipase A1 2 | Dolichovespula |
| | | | | maculata (White-face |
| | | | | hornet) (Bald-faced |
| | | | | hornet) |
| Dol m 2 | HUGA_DOLMA | P49371 | Hyaluronoglucosaminidase | Dolichovespula |
| | | | | maculata (White-face |
| | | | | hornet) (Bald-faced |
| | | | | hornet) |
| Dol m 5 | VA52_DOLMA | P10736 | Venom Allergen 5.01 | Dolichovespula |
| | | | [Precursor] | maculata (White-face |
| | | | | hornet) (Bald-faced |
| | | | | hornet) |
| Dol m 5 | VA53_DOLMA | P10737 | Venom Allergen 5.02 | Dolichovespula |
| | | | [Precursor] | maculata (White-face |
| | | | [Fragment] | hornet) (Bald-faced |
| | | | | hornet) |
| Equ c 1 | ALL1_HORSE | Q95182 | Major Allergen Equ c | Equus caballus |
| | | | 1 [Precursor] | (Horse) |
| Equ c 2 | AL21_HORSE | P81216 | Dander major | Equus caballus |
| | | | Allergen Equ c 2.0101 | (Horse) |
| | | | [Fragment] | |
| Equ c 2 | AL22_HORSE | P81217 | Dander Major | Equus caballus |
| | | | Allergen Equ c 2.0102 | (Horse) |
| | | | [Fragment] | |
| Eur m 1 | EUM1_EURMA | P25780 | Mite Group I Allergen | Euroglyphus maynei |
| | | | Eur m 1 [Fragment] | (House-dust mite) |
| Fel d 1 | FELA_FELCA | P30438 | Major Allergen I | Felis silvestris catus |
| | | | Polypeptide Chain 1 | (Cat) |
| | | | Major Form | |
| | | | [Precursor] | |
| Fel d 1 | FELB_FELCA | P30439 | Major Allergen I | Felis silvestris catus |
| | | | Polypeptide Chain 1 | (Cat) |
| | | | Minor Form | |
| | | | [Precursor] | |
| Fel d 1 | FEL2_FELCA | P30440 | Major Allergen I | Felis silvestris catus |
| | | | Polypeptide Chain 2 | (Cat) |
| | | | [Precursor] | |
| Gad c 1 | PRVB_GADCA | P02622 | Parvalbumin Beta | Gadus callarias |
| | | | | (Baltic cod) |
| Gal d 1 | IOVO_CHICK | P01005 | Ovomucoid | Gallus gallus |
| | | | [Precursor] | (Chicken) |
| Gal d 2 | OVAL_CHICK | P01012 | Ovalbumin | Gallus gallus |
| | | | | (Chicken) |
| Gal d 3 | TRFE_CHICK | P02789 | Ovotransferrin | Gallus gallus |
| | | | [Precursor] | (Chicken) |
| Gal d 4 | LYC_CHICK | P00698 | Lysozyme C | Gallus gallus |
| | | | [Precursor] | (Chicken) |
| Hel a 2 | PROF_HELAN | O81982 | Profilin | Helianthus annuus |
| | | | | (Common sunflower) |
| Hev b 1 | REF_HEVBR | P15252 | Rubber Elongation | Hevea brasiliensis |
| | | | Factor Protein | (Para rubber tree) |
| Hev b 5 | HEV5_HEVBR | Q39967 | Major Latex Allergen | Hevea brasiliensis |
| | | | Hev b 5 | (Para rubber tree) |
| Hol l 1 | MPH1_HOLLA | P43216 | Major Pollen Allergen | Holcul lanatus (Velvet |
| | | | Hol l 1 [Precursor] | grass) |
| Hor v 1 | IAA1_HORVU | P16968 | Alpha-amylase | Hordeum vulgare |
| | | | Inhibitor Bmai-1 | (Barley) |
| | | | [Precursor] | |
| | | | [Fragment] | |
| Jun a 1 | MPA1_JUNAS | P81294 | Major Pollen Allergen | Juniperus ashei |
| | | | Jun a 1 [Precursor] | (Ozark white cedar) |
| Jun a 3 | PRR3_JUNAS | P81295 | Pathogenesis-Related | Juniperus ashei |
| | | | Protein [Precursor] | (Ozark white cedar) |
| Lep d 1 | LEP1_LEPDS | P80384 | Mite Allergen Lep d 1 | Lepidoglyphus |
| | | | [Precursor] | destructor (Storage |
| | | | | mite) |
| Lol p 1 | MPL1_LOLPR | P14946 | Pollen Allergen Lol p | Lolium perenne |
| | | | 1 [Precursor] | (Perennial ryegrass) |
| Lol p 2 | MPL2_LOLPR | P14947 | Pollen Allergen Lol p | Lolium perenne |
| | | | 2-a | (Perennial ryegrass) |
| Lol p 3 | MPL3_LOLPR | P14948 | Pollen Allergen Lol p 3 | Lolium perenne |
| | | | | (Perennial ryegrass) |
| Lol p 5 | MP5A_LOLPR | Q40240 | Major Pollen Allergen | Lolium perenne |
| | | | Lol p 5a [Precursor] | (Perennial ryegrass) |
| Lol p 5 | MP5B_LOLPR | Q40237 | Major Pollen Allergen | Lolium perenne |
| | | | Lol p 5b [Precursor] | (Perennial ryegrass) |
| Mal d 1 | MAL1_MALDO | P43211 | Major Allergen Mal d 1 | Malus domestica |
| | | | | (Apple) (Malus |
| | | | | sylvestris) |
| Mer a 1 | PROF_MERAN | O49894 | Profilin | Mercurialis annua |
| | | | | (Annual mercury) |
| Met e 1 | TPM1_METEN | Q25456 | Tropomyosin | Metapenaeus ensis |
| | | | | (Greasyback shrimp) |
| | | | | (Sand shrimp) |
| Mus m 1 | MUP6_MOUSE | P02762 | Major Urinary Protein | Mus musculus |
| | | | 6 [Precursor] | (Mouse) |
| Myr p 1 | MYR1_MYRPI | Q07932 | Major Allergen Myr p | Myrmecia pilosula |
| | | | 1 [Precursor] | (Bulldog ant) |
| | | | | (Australian jumper |
| | | | | ant) |
| Myr p 2 | MYR2_MYRPI | Q26464 | Allergen Myr p 2 | Myrmecia pilosula |
| | | | [Precursor] | (Bulldog ant) |
| | | | | (Australian jumper |
| | | | | ant) |
| Ole e 1 | ALL1_OLEEU | P19963 | Major Pollen Allergen | Olea europaea |
| | | | | (Common olive) |
| Ole e 4 | ALL4_OLEEU | P80741 | Major Pollen Allergen | Olea europaea |
| | | | Ole e 4 [Fragments] | (Common olive) |
| Ole e 5 | SODC_OLEEU | P80740 | Superoxide Dismutase | Olea europaea |
| | | | [CU-ZN] [Fragment] | (Common olive) |
| Ole e 7 | ALL7_OLEEU | P81430 | Pollen Allergen Ole e | Olea europaea |
| | | | 7 [Fragment] | (Common olive) |
| Ory s 1 | MPO1_ORYSA | Q40638 | Major Pollen Allergen | Oryza sativa (Rice) |
| | | | Ory s 1 [Precursor] | |
| Par j 1 | NL11_PARJU | P43217 | Probable Nonspecific | Parietaria judaica |
| | | | Lipid-Transfer Protein | |
| | | | [Fragment] | |
| Par j 1 | NL12_PARJU | O04404 | Probable Nonspecific | Parietaria judaica |
| | | | Lipid-Transfer Protein | |
| | | | 1 [Precursor] | |
| Par j 1 | NL13_PARJU | Q40905 | Probable Nonspecific | Parietaria judaica |
| | | | Lipid-Transfer Protein | |
| | | | 1 [Precursor] | |
| Par j 2 | NL21_PARJU | P55958 | Probable Nonspecific | Parietaria judaica |
| | | | Lipid-Transfer Protein | |
| | | | 2 [Precursor] | |
| Par j 2 | NL22_PARJU | O04403 | Probable Nonspecific | Parietaria judaica |
| | | | Lipid-Transfer Protein | |
| | | | 2 [Precursor] | |
| Pha a 1 | MPA1_PHAAQ | Q41260 | Major Pollen Allergen | Phalaris aquatica |
| | | | Pha a 1 [Precursor] | |
| Pha a 5 | MP51_PHAAQ | P56164 | Major Pollen Allergen | Phalaris aquatica |
| | | | Pha a 5.1 [Precursor] | |
| Pha a 5 | MP52_PHAAQ | P56165 | Major Pollen Allergen | Phalaris aquatica |
| | | | Pha a 5.2 [Precursor] | |
| Pha a 5 | MP53_PHAAQ | P56166 | Major Pollen Allergen | Phalaris aquatica |
| | | | Pha a 5.3 [Precursor] | |
| Pha a 5 | MP54_PHAAQ | P56167 | Major Pollen Allergen | Phalaris aquatica |
| | | | Pha a 5.4 [Fragment] | |
| Phl p 1 | MPP1_PHLPR | P43213 | Pollen Allergen Phl p | Phleum pratense |
| | | | 1 [Precursor] | (Common timothy) |
| Phl p 2 | MPP2_PHLPR | P43214 | Pollen Allergen Phl p | Phleum pratense |
| | | | 2 [Precursor] | (Common timothy) |
| Phl p 5 | MP5A_PHLPR | Q40962 | Pollen Allergen Phl p | Phleum pratense |
| | | | 5a [Fragment] | (Common timothy) |
| Phl p 5 | MP5B_PHLPR | Q40963 | Pollen Allergen Phl p | Phleum pratense |
| | | | 5b [Precursor] | (Common timothy) |
| | | | [Fragment] | |
| Phl p 6 | MPP6_PHLPR | P43215 | Pollen Allergen Phl p | Phleum pratense |
| | | | 6 [Precursor] | (Common timothy) |
| Phl p 11 | PRO1_PHLPR | P35079 | Profilin 1 | Phleum pratense |
| | | | | (Common timothy) |
| Phl p 11 | PRO2_PHLPR | O24650 | Profilin 2/4 | Phleum pratense |
| | | | | (Common timothy) |
| Phl p 11 | PRO3_PHLPR | O24282 | Profilin 3 | Phleum pratense |
| | | | | (Common timothy) |
| Poa p 9 | MP91_POAPR | P22284 | Pollen Allergen Kbg | Poa pratensis |
| | | | 31 [Precursor] | (Kentucky bluegrass) |
| Poa p 9 | MP92_POAPR | P22285 | Pollen Allergen Kbg | Poa pratensis |
| | | | 41 [Precursor] | (Kentucky bluegrass) |
| Poa p 9 | MP93_POAPR | P22286 | Pollen Allergen Kbg | Poa pratensis |
| | | | 60 [Precursor] | (Kentucky bluegrass) |
| Pol a 5 | VA5_POLAN | Q05109 | Venom Allergen 5 | Polistes annularis |
| | | | [Precursor] | (Paper wasp) |
| | | | [Fragment] | |
| Pol d 5 | VA5_POLDO | P81656 | Venom Allergen 5 | Polistes dominulus |
| | | | | (European paper |
| | | | | wasp) |
| Pol e 5 | VA5_POLEX | P35759 | Venom Allergen 5 | Polistes exclamans |
| | | | | (Paper wasp) |
| Pol f 5 | VA5_POLFU | P35780 | Venom Allergen 5 | Polistes fuscatus |
| | | | | (Paper wasp) |
| Pru a 1 | PRU1_PRUAV | O24248 | Major Allergen Pru a 1 | Prunus avium |
| | | | | (Cherry) |
| Rat n 1 | MUP_RAT | P02761 | Major Urinary Protein | Rattus norvegicus |
| | | | [Precursor] | (Rat) |
| Sol i 2 | VA2_SOLIN | P35775 | Venom Allergen II | Solenopsis invicta |
| | | | [Precursor] | (Red imported fire |
| | | | | ant) |
| Sol i 3 | VA3_SOLIN | P35778 | Venom Allergen III | Solenopsis invicta |
| | | | | (Red imported fire |
| | | | | ant) |
| Sol i 4 | VA4_SOLIN | P35777 | Venom Allergen IV | Solenopsis invicta |
| | | | | (Red imported fire |
| | | | | ant) |
| Sol r 2 | VA2_SOLRI | P35776 | Venom Allergen II | Solenopsis richteri |
| | | | | (Black imported fire |
| | | | | ant) |
| Sol r 3 | VA3_SOLRI | P35779 | Venom Allergen III | Solenopsis richteri |
| | | | | (Black imported fire |
| | | | | ant) |
| Ves c 5 | VA51_VESCR | P35781 | Venom Allergen 5.01 | Vespa crabro |
| | | | | (European hornet) |
| Ves c 5 | VA52_VESCR | P35782 | Venom Allergen 5.02 | Vespa crabro |
| | | | | (European hornet) |
| Ves f 5 | VA5_VESFL | P35783 | Venom Allergen 5 | Vespula flavopilosa |
| | | | | (Yellow jacket) |
| | | | | (Wasp) |
| Ves g 5 | VA5_VESGE | P35784 | Venom Allergen 5 | Vespula germanica |
| | | | | (Yellow jacket) |
| | | | | (Wasp) |
| Ves m 1 | PA1_VESMC | P51528 | Phospholipase A1 | Vespula maculifrons |
| | | | | (Eastern yellow |
| | | | | jacket) (Wasp) |
| Ves m 5 | VA5_VESMC | P35760 | Venom Allergen 5 | Vespula maculifrons |
| | | | | (Eastern yellow |
| | | | | jacket) (Wasp) |
| Ves p 5 | VA5_VESPE | P35785 | Venom Allergen 5 | Vespula pensylvanica |
| | | | | (Western yellow |
| | | | | jacket) (Wasp) |
| Ves s 5 | VA5_VESSQ | P35786 | Venom Allergen 5 | Vespula squamosa |
| | | | | (Southern yellow |
| | | | | jacket) (Wasp) |
| Ves v 1 | PA1_VESVU | P49369 | Phospholipase A1 | Vespula vulgaris |
| | | | [Precursor] | (Yellow jacket) |
| | | | | (Wasp) |
| Ves v 2 | HUGA_VESVU | P49370 | Hyaluronoglucosaminidase | Vespula vulgaris |
| | | | | (Yellow jacket) |
| | | | | (Wasp) |
| Ves v 5 | VA5_VESVU | Q05110 | Venom Allergen 5 | Vespula vulgaris |
| | | | [Precursor] | (Yellow jacket) |
| | | | | (Wasp) |
| Ves vi 5 | VA5_VESVI | P35787 | Venom Allergen 5 | Vespula vidua |
| | | | | (Yellow jacket) |
| | | | | (Wasp) |
| Vesp m 5 | VA5_VESMA | P81657 | Venom Allergen 5 | Vespa mandarinia |
| | | | | (Hornet) |
| Zea m 1 | MPZ1_MAIZE | Q07154 | Pollen Allergen Zea | Zea mays (Maize) |
| | | | m 1 | |

In other embodiments, the amino acid sequence of the second polypeptide of the fusion molecule is defined with reference to an autoantigen sequence.

Examples of autoantigen sequences are listed in Table 2 below. Portions of the autoantigens listed in Table 2 are also suitable for use in the fusion polypeptides, wherein the portion retains at least one autoantigen epitope, and retains the ability to specifically bind the autoantibody or autoreactive T-cell receptor. For example, useful portions of the multiple sclerosis autoantigens myelin-basic-protein (amino acids 83-99), proteolipid protein (amino acids 139-151) and myelin oligodendrocyte glycoprotein (amino acids 92-106) are known, where the portions retain at least one autoantigenic epitope.

TABLE 2

| | Autoimmune | Reference and/or GenBank Accession |
|---|---|---|
| Auto-antigen | Disease(s) | No. |
| acetylcholine receptor (AChR) | myasthenia gravis | Patrick and Lindstrom, Science 180: 871-872 |
| | | (1973); Lindstrom et al., Neurology 26: 1054-1059 |
| | | (1976); Protti et al., Immunol. Today, |
| | | 15(1): 41-42 (1994); Q04844; P02708; |
| | | ACHUA1; AAD14247 |
| gravin | | Nauert et al., Curr. Biol., 7(1): 52-62 (1997); |
| | | Q02952; AAB58938 |
| titin (connectin) | | Gautel et al., Neurology 43: 1581-1585 (1993); |
| | | Yamamoto et al., Arch. Neurol., 58(6): 869-870 |
| | | (2001); AAB28119 |
| neuronal voltage-gated | Lambert-Eaton myasthenic | Rosenfeld et al., Ann. Neurol., 33(1): 113-120 |
| calcium channel | syndrome | (1993); A48895 |
| CNS myelin-basic-protein | multiple sclerosis | Warren et al., Proc. Natl. Acad. Sci. USA |
| (MBP), MBP₈₃₋₉₉ epitope | | 92: 11061-11065 [1995]; Wucherpfennig et |
| | | al., J. Clin. Invest., 100(5): 1114-1122 [1997]; |
| | | Critchfield et al., Science 263: 1139-1143 |
| | | [1994]; Racke et al., Ann. Neurol., 39(1): 46-56 |
| | | [1996]; XP_040888; AAH08749; P02686 |
| proteolipid protein (PLP), | | XP_010407 |
| PLP₁₃₉₋₁₅₁ epitope | | |
| PLP₁₇₈₋₁₉₁ epitope | | |
| myelin oligodendrocyte | | XP_041592 |
| glycoprotein (MOG), | | |
| MOG₉₂₋₁₀₆ epitope | | |
| αβ-crystallin | | Van Noort et al., Nature 375: 798 (1995); Van |
| | | Sechel et al., J. Immunol., 162: 129-135 |
| | | (1999); CYHUAB |
| myelin-associated | | Latov, Ann. Neurol., 37(Suppl. 1): S32-S42 |
| glycoprotein (MAG), Po | | (1995); Griffin, Prog. Brain Res., 101: 313-323 |
| glycoprotein and PMP22 | | (1994); Rose and MacKay (Eds.), The |
| | | Autoimmune Diseases, Third Edition, |
| | | Academic Press, p. 586-602 [1998]; |
| | | XP_012878; P20916 |
| 2′,3′-cyclic nucleotide 3′- | | P09543; JC1517 |
| phosphohydrolase (CNPase) | | |
| glutamic acid decarboxylase | type-I (insulin dependent) | Yoon et al., Science 284: 1183-1187 [1999]; |
| (GAD), and various isoforms | diabetes mellitus, also Stiff-Man | Nepom et al., Proc. Natl. Acad. Sci. USA |
| (e.g., 65 and 67 kDa isoforms) | Syndrome (GAD) and other | 98(4): 1763-1768 [2001]; Lernmark, J. Intern. |
| | diseases (GAD) | Med., 240: 259-277 [1996]; B41935; A41292; |
| | | P18088; Q05329 |
| insulin | | Wong et al., Nature Med., 5: 1026-1031 |
| | | [1999]; Castano et al., Diabetes 42: 1202-1209 |
| | | (1993) |
| 64 kD islet cell antigen/ | | Rabin et al., Diabetes 41: 183-186 (1992); |
| tyrosine phosphatase-like islet | | Rabin et al., J. Immunol., 152: 3183-3187 |
| cell antigen-2 (IA-2, also | | (1994); Lan et al., DNA Cell Biol., 13: 505-514 |
| termed ICA512) | | (1994) |
| phogrin (IA-2β) | | Wasmeier and Hutton, J. Biol. Chem., |
| | | 271: 18161-18170 (1996); Q92932 |
| type II collagen | rheumatoid arthritis | Cook et al., J. Rheumatol., 21: 1186-1191 |
| | | (1994); and Terato et al., Arthritis |
| | | Rheumatol., 33: 1493-1500 (1990) |
| human cartilage gp39 | | P29965; XP_042961 |
| (HCgp39) | | |
| gp130-RAPS | | P40189; BAA78112 |
| scl-70 antigen/topoisomerase-I | scleroderma (systemic sclerosis), | Douvas et al., J. Biol. Chem., 254: 10514-10522 |
| | various connective tissue | (1979); Shero et al., Science 231: 737-740 |
| | diseases | (1986); P11387 |
| topoisomerase II (α/β) | | Meliconi et al., Clin. Exp. Immunol., |
| | | 76(2): 184-189(1989); XP_008649; |
| | | NP_001059; Q02880 |
| type I collagen | | Riente et al., Clin. Exp. Immunol., |
| | | 102(2): 354-359 (1995); XP_037912 |
| fibrillarin, U3-small nuclear | | Arnett et al., Arthritis Rheum., 39: 151-160 |
| protein (snoRNP) | | (1996) |
| Jo-1 antigen/aminoacyl | polymyositis, dermatomyositis, | Mathews and Bernstein, Nature 304: 177-179 |
| histidyl-tRNA synthetase | interstitial lung disease, | (1983); Bernstein, Bailliere's Clin. Neurol., |
| PL-7 antigen/threonyl tRNA | Raynaud's phenomenon, also | 2: 599-616 (1993); Targoff, J. Immunol., |
| synthetase | scleroderma (PM-scl) | 144(5): 1737-1743 (1990); Targoff, J. Invest. |
| PL-12 antigen/alanyl tRNA | | Dermatol., 100: 116S-123S (1995); Rider and |
| synthetase | | Miller, Clin. Diag. Lab. Immunol., 2: 1-9 |
| EJ antigen/glycyl-tRNA | | (1995); Targoff, J. Invest. Dermatol., |
| synthetase | | 100: 116S-123S (1995); von Muhlen and Tan, |
| OJ antigen/NJ antigen | | Semin. Arthritis Rheum., 24: 323-358 (1995); |
| isoleucyl-tRNA synthetase | | Targoff et al., J. Clin. Invest., 84: 162-172 |
| signal recognition particle | | (1989) |
| (SRP) | | |
| Mi-2 helicase | | |
| PM-scl proteins (75 kDa, 100 kDa) | | |
| KJ antigen | | |
| Fer antigen/ | | |
| elongation fractor 1α | | |
| Mas antigen/ | | |
| tRNA^{Ser} | | |
| type IV collagen α3 chain | Goodpasture syndrome | Hellmark et al., Kidney Int., 46: 823-829 |
| | | (1994); Q01955 |
| Smith (Sm) antigens and | systemic lupus erythematosus, | Lerner and Steitz, Proc. Natl. Acad. Sci. USA |
| snRNP's, including snRNPs | mixed connective tissue disease | 76: 5495-5499 (1979); Reuter et al., Eur. J. |
| D1, D2, D3, B, B′, B3 (N), E, | (MCTD), progressive systemic | Immunol., 20: 437-440 (1990); Petersson et al., |
| F, and G, as found in RNP | sclerosis, rheumatoid arthritis, | J. Biol. Chem., 259: 5907-5914 (1984) |
| complexes U1, U2, U4/6, and | discoid lupus erythematosus, | |
| U5. | Sjögren's syndrome | |
| nRNP U1-snRNP complex, | | Klein et al., Clin. Exp. Rheumatol., 15: 549-560 |
| including subunits U1-70 kD, | | (1997) |
| A and C. | | |
| deoxyribonucleic acid (DNA), | systemic lupus erythematosus | Pisetsky, Curr. Top. Microl. Immunol., |
| double-stranded B-form | | 247: 143-155 (2000); Radic et al., Crit. Rev. |
| deoxyribonucleic acid (DNA), | | Immunol., 19(2): 117-126 (1999) |
| denatured/single-stranded | | |
| Cyclin A | autoimmune hepatic disease, and | Strassburg et al., Gastroenterology 111: 1582-1592 |
| | other diseases | (1996); Strassburg et al., J. Hepatol., |
| | | 25(6): 859-866 (1996) |
| Ro (SS-A) antigens | Sjögren's syndrome, systemic | Tan, Adv. Immunol., 44: 93-(1989); |
| 52 kDa | and cutaneous lupus | McCauliffe and Sontheimer, J. Invest. |
| 60 kDa | erythematosis, rheumatoid | Dermatol., 100: 73S-79S (1993); Wolin and |
| | arthritis, neonatal lupus | Steitz, Proc. Natl. Acad. Sci. USA 81: 1996-2000 |
| | syndrome, polymyositis, | (1984); Slobbe et al., Ann. Med. |
| | progressive systemic sclerosis, | Interne., 142: 592-600 (1991); AAB87094; |
| | primary biliary cirrhosis | U01882; P10155 |
| La (SS-B) antigen | Sjögren's syndrome, neonatal | Manoussakis et al., Scan. J. Rheumatol., |
| | lupus syndrome, systemic lupus | 61: 89-92 (1986); Harley et al., Arthritis |
| | erythematosis | Rheum., 29: 196-206 (1986); Slobbe et al., |
| | | Ann. Med. Interne., 142: 592-600 (1991); |
| | | P05455 |
| proteinase-3 (serine | Wegener's granulomatosis, | Ledemann et al., J. Exp. Med., 171: 357-362 |
| proteinase)/cytoplasmic | systemic vasculitis, microscopic | (1990); Jenne et al., Nature 346: 520 (1990); |
| neutrophil antigen (cANCA)/ | polyangiitis, idiopathic crescentic | Gupta et al., Blood 76: 2162 (1990); P24158 |
| myeloblastin | glomerulonephritis, Churg- | |
| | Strauss syndrome, polyarteritis | |
| | nodosa | |
| myeloperoxidase/nuclear or | systemic lupus erythrematosus/ | Lee et al., Clin. Exp. Immunol., 79: 41-46 |
| perinuclear neutrophil antigen | antiphospholipid syndrome | (1990); Cohen Tervaert et al., Arthr. Rheum., |
| (pANCA) | (APS)/thrombocytopenia/ | 33: 1264-1272 (1990); Gueirard et al., J. |
| | recurrent thromboembolic | Autoimmun., 4: 517-527 (1991); Ulmer et al., |
| | phenomenon | Clin. Nephrol., 37: 161-168 (1992); P05164 |
| β₂-glycoprotein-1 (aka | antiphospholipid/cofactor | McNeil et al., Proc. Natl. Acad. Sci. USA |
| apolipoprotein H) | syndromes, autoimmune | 87: 4120-4124 (1990) |
| cardiolipin, | gastritis/type A chronic atrophic | Alarcon-Segovia and Cabral, Lupus 5: 364-367 |
| phosphatidylcholine, and | gastritis/pernicious anaemia | (1996); and Alarcon-Segovia and Cabral, J. |
| various anionic phospholipids | | Rheumatol., 23: 1319-1322 (1996) |
| parietal cell antigen; H⁺/K⁻ | autoimmune gastritis, type A | Karlsson et al., J. Clin. Invest., 81(2): 475-479 |
| ATPase gastric proton pump α | chronic atrophic gastritis, | (1988); Burman et al., Gastroenterology |
| & β subunits | pernicious anaemia | 96(6): 1434-1438 (1989); Toh et al., Proc. |
| | | Natl. Acad. Sci. USA 87(16): 6418-6422 |
| | | (1990) |
| thyroglobulin (TG); TG₁₁₄₉₋₁₂₅₀ | Hashimoto's thyroidosis, primary | Malthiery and Lissitzky, Eur. J. Biochem., |
| | myxedema, subacute thyroiditis | 105: 491-498 (1987); Henry et al., Eur. J. |
| | | Immunol., 22: 315-319 (1992); Prentice et al., |
| | | J. Clin. Endocrinol. Metab., 80: 977-986 |
| | | (1995) |
| thyroid peroxidase (TPO); | | McLachlan and Rapoport, Endocr. Rev., |
| TPO₅₉₀₋₆₇₅ and TPO₆₅₁₋₇₅₀ | | 13: 192-206 (1992); McLachlan and Rapoport, |
| | | Clin. Exp. Immunol., 101: 200-206 (1995); |
| | | Tonacchera et al., Eur. J. Endocrinol., |
| | | 132: 53-61 (1995) |
| thyroid-stimulating hormone | Graves' disease (thyrotoxicosis) | Weetman and McGregor, Endocr. Rev., |
| receptor (TSH-R, also termed | and myxedema, hyperactive | 15: 788-830 (1994) |
| thyrotropin) | thyroid disease, Hashimoto's | |
| | thyroiditis | |
| desmosomal proteins; | pemphigus blistering disorders, | Korman et al., N. Engl. Jour. Med., 321: 631-635 |
| desmoglein-1 | and other cutaneous diseases | (1989); Amagi et al., Cell 67: 869-877 |
| desmoglein-3 | | (1991); Koulu et al., J. Exp. Med., 160: 1509-1518 |
| | | (1984); Stanley et al., J. Immunol., |
| | | 136: 1227-1230 (1986); Cozzani et al., Eur. J. |
| | | Dermatol., 10(4): 255-261 (2000) |
| hemidesmosome proteins | | Diaz et al., J. Clin. Invest., 86: 1088-1094 |
| BP180 (also known as BPAG2 | | (1990); Giudice et al., J. Invest. Dermatol., |
| and type XVII collagen) and | | 99: 243-250 (1992); Stanley et al., J. Clin. |
| BP230 (BPAG1) | | Invest., 82: 1864-1870 (1988) |
| type VII collagen | | Gammon et al., J. Invest. Dermatol., 84: 472-476 |
| | | (1985) |
| mitochondrial pyruvate | primary biliary cirrhosis, | Gershwin et al., J. Immunol., 138: 3525-3531 |
| dehydrogenase complex | autoimmune hepatitis, systemic | (1987); Moteki et al., Hepatology (Baltimore), |
| (PDC) E1α decarboxylase | sclerosis | 23: 436-444 (1996); Surh et al., Hepatology |
| mitochondrial E1β | | (Baltimore), 9: 63-68 (1989); and Yeaman et |
| decarboxylase | | al., Lancet 1: 1067-1070 (1988); Jones et al., |
| mitochondrial | | J. Clin. Pathol., 53(11): 813-821 (2000); |
| PDC-E2 acetyltransferase | | Mackay et al., Immunol. Rev., 174: 226-237 |
| mitochondrial protein X | | (2000) |
| mitochondrial branched chain | | |
| 2-oxo acid dehydrogenase | | |
| (BCOADC) E2 subunit | | |
| PDC-E2 (mitochondrial | | |
| pyruvate dehydrogenase | | |
| dehydrolipoamide | | |
| acetyltransferase) | | |
| 2-oxoglutarate dehydrogenase | | |
| (OGDC); E2 succinly | | |
| transferase | | |
| chromosomal centromere | systemic sclerosis | Earnshaw and Rothfield, Chromosoma 91(3-4): |
| proteins CENP-A, B, C and F | | 313-321 (1985) |
| coilin/p80 | autoimmune dermatological | Andrade et al., J. Exp. Med., 173(6): 1407-1419 |
| | disorders, and other diseases | (1991); Muro, J. Dermatol. Sci., |
| | | 25(3): 171-178 (2001); S50113 |
| HMG proteins | systemic lupus erythematosus, | Bustin et al., Science 215(4537): 1245-1247 |
| HMG-1 | drug induced lupus, scleroderma, | (1982); Vlachoyiannopoulos et al., J. |
| HMG-2 | autoimmune hepatitis | Autoimmun., 7(2): 193-201 (1994); Somajima |
| HMG-14 | | et al., Gut 44(6): 867-873 (1999); Ayer et al., |
| HMG-17 | | Arthritis Rheum., 37(1): 98-103 (1994) |
| Histone proteins H1, H2A, | systemic lupus erythrematosus, | Shen et al., Clin. Rev. Allergy Immunol., |
| H2B, H3 and H4 | drug induced lupus, rheumatoid | 16(3): 321-334 (1998); Burlingame and Rubin, |
| | arthritis, and other diseases | Mol. Biol. Rep., 23(3-4): 159-166 (1996) |
| Ku antigen (p70/p80) | systemic sclerosis, systemic | Yaneva et al., Clin. Exp. Immunol., 76: 366-372 |
| and | lupus erythrematosus, mixed | (1989); Mimori et al., J. Biol. Chem., |
| DNA-PK catayltic subunit | connective tissue diseases, | 261(5): 2274-2278 (1986); Tuteja and Tuteja, |
| | dermatomyositis, and other | Crit. Rev. Biochem. Mol. Biol., 35(1): 1-33 |
| | diseases | (2000); Satoh et al., Clin. Exp. Immunol., |
| | | 105(3): 460-467 (1996) |
| NOR-90/hUBF | systemic sclerosis | Dick et al., J. Rheumatol., 22: 67-72 (1995); |
| | | Rodriguez-Sanchez et al., J. Immunol., |
| | | 139(8): 2579-2584 (1987) |
| Proliferating cell nuclear | systemic lupus erythrematosus, | Takeuchi et al., Mol. Biol. Rep., 23(3-4): 243-246 |
| antigen (PCNA) | and other diseases | (1996); Fritzler et al., Arthritis Rheum., |
| | | 26(2): 140-145 (1983); P12004 |
| ribosomal RNP proteins (“P- | systemic lupus erythrematosus | Elkon et al., J. Exp. Med., 162(2): 459-471 |
| antigens”) P0, P1 and P2 | | (1985); Bonfa et al., J. Immunol., |
| | | 140(10): 3434-3437 (1988) |
| Ra33/hnRNP A2 | rheumatoid arthritis | Hassfeld et al., Arthritis Rheum., 32(12): 1515-1520 |
| | | (1989); Steiner et al., J. Clin. Invest., |
| | | 90(3): 1061-1066 (1992) |
| SP-100 | undifferentiated connective tissue | Szostecki et al., Clin. Exp. Immunol., |
| | diseases (UCTD), Sjogren's | 68(1): 108-116 (1987) |
| | syndrome, primary biliary | |
| | cirrhosis and other disorders | |
| S-antigen/interphotoreceptor | uveitis/uveoretinitis | Dua et al., Curr. Eye Res., 11: 59-65 (1992) |
| retinoid binding protein | | |
| (IRBP) | | |
| annexin XI | rheumatiod arthritis, systemic | Misaki et al., J. Biol. Chem., 269(6): 4240-4246 |
| (56K autoantigen) | lupus erythematosus, Sjögren's | (1994) |
| | syndrome | |
| hair follicle antigens | alopecia (e.g., alopecia areata) | McElwee et al., Exp. Dermatol., 8(5): 371-379 |
| | | (1999) |
| human tropomyosin isoform 5 | ulcerative colitis | Das et al., J. Immunol., 150(6): 2487-2493 |
| (hTM5) | | (1993) |
| cardiac myosin | myocarditis and cardiomyopathy | Caforia et al., Circulation 85: 1734-1742 |
| | and related diseases | (1992); Neumann et al., J. Am. Coll. Cardiol., |
| | | 16: 839-846 (1990) |
| laminin | | Wolff et al., Am. Heart Jour., 117: 1303-1309 |
| | | (1989) |
| β₁-adrenergic receptors | | Limas et al., Circ. Res., 64: 97-103 (1989) |
| mitochondrial adenine | | Schultheiss et al., Ann. NY Acad. Sci., 488: 44-64 |
| nucleotide translocator (ANT) | | (1986) |
| mitochondrial branched-chain | | Ansari et al., J. Immunol., 153(10): 4754-4765 |
| ketodehydrogenase (BCKD) | | (1994) |
| eukaryotic elongation factor | Felty's syndrome/autoimmune | Ditzel et al., Proc. Natl. Acad. Sci. USA |
| 1A-1 (eEF1A-1) | neutropenia | 97(16): 9234-9239 [2000] |
| glycoprotein gp70 (viral | systemic lupus erythematosus | Haywood et al., J. Immunol., 167(3): 1728-1733 |
| antigen) | | (2001) |
| early endosome antigen-1 | subacute systemic lupus | Mu et al., J. Biol. Chem., 270(22): 13503-13511 |
| (EEA1) | erythematosus | (1995); Stenmark et al., J. Biol. Chem., |
| | | 271(39): 24048-24054 (1996) |
| 21-hydroxylase | Addison's Disease, types I and II | Winqvist, Lancet 339: 1559-1562 (1992); |
| | autoimmune polyglandular | Bednarek et al., FEBS Lett., 309: 51-55 (1992) |
| | syndrome (APS) | |
| calcium sensing receptor (Ca- | hypoparathyroidism | Brown et al., Nature 366: 575-580 (1993); Li |
| SR) | | et al., J. Clin. Invest., 97: 910-914 (1996) |
| tyrosinase | vitiligo | Song et al., Lancet 344: 1049-1052 (1994) |
| tissue transgluaminase | celiac disease, gluen-sensitive | Dieterich et al., Nat. Med., 3(7): 797-801 |
| | enteropathy | (1997); and Schuppan et al., Ann. NY Acad. |
| | | Sci., 859: 121-126 (1998) |
| keratin proteins | inflammatory arthritis/ | Borg, Semin. Arthritis Rheum., 27(3): 186-195 |
| | rheumatoid arthritis | (1997) |
| poly (ADP-ribose) polymerase | systemic lupus erythematosus, | Muller et al., Clin. Immunol. Immunopathol., |
| (PARP) | Sjogren's syndrome, and other | 73(2): 187-196 (1994); Yamanaka et al., J. |
| | diseases | Clin. Invest., 83(1): 180-186 (1989) |
| nucleolar proteins | systemic lupus erythematosus, | Li et al., Arthritis Rheum., 32(9): 1165-1169 |
| B23/numatrin | and other diseases | (1989); Zhang et al., Biochem. Biophys. Res. |
| | | Commun., 164: 176-184 (1989); AAA36385 |
| erythrocyte surface antigens/ | autoimmune hemolytic anemia | Barker and Elson, Vet. Immunol. |
| glycophorins | | Immunopathol., 47(3-4): 225-238 (1995) |
| RNA polymerase I subunits | systemic sclerosis/scleroderma, | Hirakata et al., J. Clin. Invest., 91: 2665-2672 |
| RNA polymerase II subunits | and other diseases | (1993); and Kuwana et al., J. Clin. Invest., |
| RNA polymerase III subunits | | 91: 1399-1404 (1993) |
| Th/To (7-2 RNP; also known | | Gold et al., Science 245(4924): 1377-1380 |
| as RNase MRP) | | (1989); and Okano and Medsger, Arthritis |
| | | Rheum., 33(12): 1822-1828 (1990) |
| nuclear mitotic apparatus | various connective tissue | Andrade et al., Arthritis Rheum., 39(10): 1643-1653 |
| proteins (NuMA proteins) | diseases | (1996); Price et al., Arthritis Rheum., |
| | | 27(7): 774-779 (1984) |
| nuclear lamins A, B and C | various hepatic and connective | Hill et al., Aust. NZ J. Med., 26(2): 162-166 |
| | tissue autoimmune diseases, and | (1996); Lassoued et al., Ann. Intern. Med., |
| | other diseases | 108(6): 829-833 (1988) |
| 210-kDa glycoprotein (gp210) | primary biliary cirrhosis | Nesher et al., Semin. Arthritis Rheum., |
| | | 30(5): 313-320 (2001); Courvalin and |
| | | Worman, Semin. Liver Dis., 17(1): 79-90 |
| | | (1997) |
| pericentriolar material protein- | scleroderma, and possibly other | Balczon et al., J. Cell Biol., 124(5): 783-793 |
| 1 (PCM-1) | diseases | (1994); Mack et al., Arthritis Rheum., |
| | | 41(3): 551-558 (1998) |
| platelet surface antigens/ | autoimmune thromocytopenia | McMillan, Transfus. Med. Rev., 4: 136-143 |
| glycoproteins IIb/IIIa and | purpura | (1990) |
| Ib/IX | | |
| golgins (e.g., 95 and 160-kDa | various | Fritzler et al., J. Exp. Med., 178(1): 49-62 |
| species) | | (1993) |
| F-actin | autoimmune hepatitis and | Czaja et al., Hepatology (Baltimore) 24: 1068-1073 |
| | primary biliary cirrhosis (UGT-1 | (1996) |
| cytochrome P-450 superfamily | and mitochondrial enzymes) | Gueguen et al., Biochem, Biophys. Res. |
| proteins, most specifically | | Commun., 159: 542-547 (1989); Manns et al., |
| 2D6; epitopes: 2D6₂₅₇₋₂₆₉, | | J. Clin. Invest., 83: 1066-1072 (1989); Zanger |
| 2D6₃₂₁₋₃₅₁, 2D6₃₇₃₋₃₈₉, and | | et al., Proc. Natl. Acad. Sci. USA 85: 8256-8260 |
| 2D6₄₁₉₋₄₂₉. Also, P-450 | | (1988); Rose and MacKay (Eds.), The |
| proteins 1A2, 2B, 2C9, 2C11, | | Autoimmune Diseases, Third Edition, |
| 2E, 3A1, c21, scc, and c17a. | | Academic Press, Ch.26 “Autoimmune |
| | | Diseases: The Liver,” p.511-544 [1998] |
| UDP-glucuronosyltransferase | | Strassburg et al., Gastroenterology 111: 1582-1592 |
| family proteins (UGT-1 and | | (1996) |
| UGT-2) | | |
| asialoglycoprotein receptor | | Treichel et al., Hepatology (Baltimore) |
| (ASGP-R) | | 11: 606-612 (1990) |
| amphiphysin | Stiff-Man syndrome | David et al., FEBS Lett., 351: 73-79 (1994) |
| glutamate receptor Glu R3 | Rasmussen's encephalitis | Rogers et al., Science 265: 648-651 (1994) |
| human gangliosides, especially | Guillain-Barré Syndrome, and | reviewed in Hartung et al., Muscle Nerve |
| GM₁, and also GD1a, N- | related neuronal syndromes (e.g., | 18: 137-153 (1995) and Rose and MacKay |
| acetylgalactosaminyl-GD1a, | Miller-Fisher Syndrome); and | (Eds.), The Autoimmune Diseases, Third |
| GD1b, GQb1, LM1, GT1b and | autoimmune diabetes | Edition, Academic Press, p. 586-602 [1998] |
| asialo-GM₁. | (sulphatide) | |
| sulphatide | | |
| (3′-sulphogalactosylceramide) | | |

It is not intended that useful autoantigen sequences be limited to those sequences provided in Table 2, as methods for the identification of additional autoantigens are known in the art, e.g., SEREX techniques (serological identification of antigens by recombinant expression cloning), where expression libraries are screened using autoimmune sera probes (Bachmann et al., Cell 60:85-93 [1990]; and Pietromonaco et al., Proc. Natl. Acad. Sci. USA 87:1811-1815 [1990]; Folgori et al., EMBO J., 13:2236-2243 [1994]). Similarly, it is not intended that the autoimmune diseases that can be treated using the compositions and methods of the invention be limited to the diseases listed in Table 2, as additional diseases which have autoimmune etiologies will be identified in the future.

2. Preparation of the Vaccine

Suitable vectors are prepared using standard techniques of recombinant DNA technology, and are, for example, described in “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991). Isolated plasmids and DNA fragments are cleaved, tailored, and ligated together in a specific order to generate the desired vectors. After ligation, the vector containing the gene to be expressed is transformed into a suitable host cell.

Host cells can be any eukaryotic or prokaryotic hosts known for expression of heterologous proteins. Accordingly, the polypeptides of the present invention can be expressed in eukaryotic hosts, such as eukaryotic microbes (yeast) or cells isolated from multicellular organisms (mammalian cell cultures), plants and insect cells. Examples of mammalian cell lines suitable for the expression of heterologous polypeptides include monkey kidney CV1 cell line transformed by SV40 (COS-7, ATCC CRL 1651); human embryonic kidney cell line 293S (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CV1-76, ATCC CCL 70); African green monkey cells (VERO-76, ATCC CRL-1587); human cervical carcinoma cells (HELA, ATCC CCL 2); canine kidney cells (MDCK, ATCC CCL 34); human lung cells (W138, ATCC CCL 75); and human liver cells (Hep G2, HB 8065). In general myeloma cells, in particular those not producing any endogenous antibody, e.g. the non-immunoglobulin producing myelome cell line SP2/0, may be used for the production of the antibody herein.

Eukaryotic expression systems employing insect cell hosts may rely on either plasmid or baculoviral expression systems. The typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedrin promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Invitrogen (Bac-N-Blue™), Clontech (BacPAK™ Baculovirus Expression System), Life Technologies (BAC-TO-BAC™), Novagen (Bac Vector System™), Pharmingen and Quantum Biotechnologies). Another insect cell host is common fruit fly, Drosophila melanogaster, for which a transient or stable plasmid based transfection kit is offered commercially by Invitrogen (The DES™ System).

Saccharomyces cerevisiae is the most commonly used among lower eukaryotic hosts. However, a number of other genera, species, and strains are also available and useful herein, such as Pichia pastoris (EP 183,070; Sreekrishna et al., J. Basic Microbiol. 28:165-278 (1988)). Yeast expression systems are commercially available, and can be purchased, for example, from Invitrogen (San Diego, Calif.). Other yeasts suitable for bi-functional protein expression include, without limitation, Kluyveromyces hosts (U.S. Pat. No. 4,943,529), e.g. Kluyveromyces lactis; Schizosaccharomyces pombe (Beach and Nurse, Nature 290:140 (1981); Aspergillus hosts, e.g. A. niger (Kelly and Hynes, EMBO J. 4:475-479 (1985])) and A. nidulans (Ballance et al., Biochem. Biophys. Res. Commun. 112:284-289 (1983)), and Hansenula hosts, e.g. Hansenula polymorpha. Yeasts rapidly grow on inexpensive (minimal) media, the recombinant can be easily selected by complementation, expressed proteins can be specifically engineered for cytoplasmic localization or for extracellular export, and they are well suited for large-scale fermentation.

Prokaryotes may be hosts for the initial cloning steps, and are useful for rapid production of large amounts of DNA, for production of single-stranded DNA templates used for site-directed mutagenesis, for screening many mutants simultaneously, and for DNA sequencing of the mutants generated. E. coli strains suitable for the production of the peptides of the present invention include, for example, BL21 carrying an inducible T7 RNA polymerase gene (Studier et al., Methods Enzymol. 185:60-98 (1990)); AD494 (DE3); EB105; and CB (E. coli B) and their derivatives; K12 strain 214 (ATCC 31,446); W3110 (ATCC 27,325); X1776 (ATCC 31,537); HB101 (ATCC 33,694); JM101 (ATCC 33,876); NM522 (ATCC 47,000); NM538 (ATCC 35,638); NM539 (ATCC 35,639), etc. Many other species and genera of prokaryotes may be used as well. Indeed, the peptides of the present invention can be readily produced in large amounts by utilizing recombinant protein expression in bacteria, where the peptide is fused to a cleavable ligand used for affinity purification.

Suitable promoters, vectors and other components for expression in various host cells are well known in the art and are disclosed, for example, in the textbooks listed above.

Whether a particular cell or cell line is suitable for the production of the polypeptides herein in a functionally active form, can be determined by empirical analysis. For example, an expression construct comprising the coding sequence of the desired molecule may be used to transfect a candidate cell line. The transfected cells are then grown in culture, the medium collected, and assayed for the presence of secreted polypeptide. The product can then be quantitated by methods known in the art, such as by ELISA.

Alternatively, the entire molecule, may be prepared by chemical synthesis, such as solid phase peptide synthesis. Such methods are well known to those skilled in the art. In general, these methods employ either solid or solution phase synthesis methods, described in basic textbooks, such as, for example, J. M. Stewart and J. D. Young, Solid Phase Peptide Synthesis, 2nd Ed., Pierce Chemical Co., Rockford, Ill. (1984) and G. Barany and R. B. Merrifield, The Peptide: Analysis Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

The molecules of the present invention may include amino acid sequence variants. Such amino acid sequence variants can be produced by expressing the underlying DNA sequence in a suitable recombinant host cell, or by in vitro synthesis of the desired polypeptide, as discussed above. The nucleic acid sequence encoding a polypeptide variant may be prepared by site-directed mutagenesis of the nucleic acid sequence encoding the corresponding native (e.g. human) polypeptide. Site-directed mutagenesis using polymerase chain reaction (PCR) amplification may be used. (see, for example, U.S. Pat. No. 4,683,195 issued Jul. 28, 1987; and Current Protocols In Molecular Biology, Chapter 15 (Ausubel et al., ed., 1991). Other site-directed mutagenesis techniques are also well known in the art and are described, for example, in the following publications: Current Protocols In Molecular Biology, supra, Chapter 8; Molecular Cloning: A Laboratory Manual., 2nd edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

The polypeptides of the invention can also be prepared by the combinatorial peptide library method disclosed, for example, in International Patent Publication PCT WO 92/09300. This method is particularly suitable for preparing and analyzing a plurality of molecules, that are variants of given predetermined sequences, and is, therefore, particularly useful in identifying polypeptides with improved biological properties, which can then be produced by any technique known in the art, including recombinant DNA technology and/or chemical synthesis.

3. Therapeutic Uses of the Vaccines of the Invention

The present invention specifically provides a new therapeutic DNA vaccine strategy for prevention and treatment of IgE mediated or so called immediate hypersensitivity diseases. In particular, the invention provides compounds for use in the prevention and treatment of allergic diseases where there is a Th2 polarized response and induction of allergic inflammation.

4. Nature of the Diseases Targeted

Following the Gell and Coombs Classification, allergic reactions are classified depending on the type of immune response induced and the resulting tissue damage that develops as a result of reactivity to an antigen. A Type I reaction (immediate hypersensitivity) occurs when an antigen (called an allergen in this case) entering the body encounters mast cells or basophils which are sensitized as a result of IgE to that antigen being attached to its high-affinity receptor, FcεRI. Upon reaching the sensitized mast cell, the allergen cross-links IgE bound to FcεRI, causing an increase in intracellular calcium (Ca²⁺) that triggers the release of pre-formed mediators, such as histamine and proteases, and newly synthesized, lipid-derived mediators such as leukotrienes and prostaglandins. These autocoids produce the acute clinical symptoms of allergy. The stimulated basophils and mast cells will also produce and release proinflammatory mediators, which participate in the acute and delayed phase of allergic reactions. It is also clear now that other parts of the immune system, e.g. T cells and NKT cells play an active role in the overall immediate hypersensitivity reactions.

As discussed before, a large variety of allergens have been identified so far, and new allergens are identified, cloned and sequenced practically every week.

Ingestion of an allergen results in gastrointestinal and systemic allergic reactions. The most common food allergens involved are peanuts, shellfish, milk, fish, soy, wheat, egg and tree nuts such as walnuts. In susceptible people, these foods can trigger a variety of allergic symptoms, such as nausea, vomiting, diarrhea, urticaria, angioedema, asthma and full-blown anaphylaxis. Inhalation of airborne allergens results in allergic rhinitis and allergic asthma, which can be acute or chronic depending on the nature of the exposure(s). Exposure to airborne allergens in the eye results in allergic conjunctivitis. Common airborne allergens includes pollens, mold spores, dust mites and other insect proteins that are the most frequent cause of seasonal hay fever and allergic asthma.

Cutaneous exposure to an allergen, e.g. natural rubber latex proteins as found in latex gloves, may result in local allergic reactions manifest as hives (urticaria) at the places of contact with the allergen as well as generalized reactions.

Systemic exposure to an allergen such as occurs with a bee sting, the injection of penicillin, or the use of natural rubber latex (NRL) gloves inside a patient during surgery may result in, cutaneous, gastrointestinal and respiratory reactions up to and including airway obstruction and full blown anaphylaxis. Hymenoptera stings are insects that commonly cause allergic reactions, often leading the anaphylactic shock. Examples include various bees including honeybees, yellow jackets, yellow hornets, wasps and white-faced hornets. Certain ants known as fire ants (Solenopsis invicta) are an increasing cause of allergy in the US as they expand their range in this country. Proteins in NRL gloves have become an increasing concern to health care workers and patients and at present, there is no successful form of therapy for this problem except avoidance.

5. Uses of Compounds for Targeted Diseases

The compounds disclosed herein can be used to acutely or chronically inhibit IgE mediated reaction to major environmental and occupational allergens, and in particular can be used to provide protection for allergy vaccination (immunotherapy) to induce a state of non-allergic reactivity (so called “allergic tolerance) during treatment for specific allergens and can also have a prophylactic effect against allergic disease by preventing allergic sensitization to environmental and occupational allergens when administered to at-risk individuals (e.g., those at genetic risk of asthma and those exposed to occupational allergens in the workplace).

6. Compositions and Formulations of the Invention

For therapeutic uses, including prevention, the compounds of the invention can be formulated as pharmaceutical compositions in admixture with pharmaceutically acceptable carriers or diluents. Methods for making pharmaceutical formulations are well known in the art.

Techniques and formulations generally may be found in Remington's Pharmaceutical Sciences, 18th Edition, Mack Publishing Co., Easton, Pa. 1990. See, also, Wang and Hanson “Parenteral Formulations of Proteins and Peptides: Stability and Stabilizers”, Journal of Parenteral Science and Technology, Technical Report No. 10, Supp. 42-2S (1988). A suitable administration format can best be determined by a medical practitioner for each patient individually.

Pharmaceutical compositions of the present invention can comprise a vaccine of the present invention along with conventional carriers and optionally other ingredients.

Suitable forms, in part, depend upon the use or the route of entry, for example oral, transdermal, inhalation, or by injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms that prevent the agent or composition from exerting its effect.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical compositions can be administered by different routes including, but not limited to, oral, intravenous, intra-arterial, intraperitoneal, subcutaneous, intranasal or intrapulmonary routes. The desired isotonicity of the compositions can be accomplished using sodium chloride or other pharmaceutically acceptable agents such as dextrose, boric acid, sodium tartrate, propylene glycol, polyols (such as mannitol and sorbitol), or other inorganic or organic solutes.

For systemic administration, injection may be used e.g. intradermal, subcutaneous, intramuscular, intravenous, etc. For injection, the compounds of the invention are formulated in liquid solutions, such as in physiologically compatible buffers such as Hank's solution or Ringer's solution. Alternatively, the compounds of the invention are formulated in one or more excipients (e.g., propylene glycol) that are generally accepted as safe as defined by USP standards. They can, for example, be suspended in an inert oil, suitably a vegetable oil such as sesame, peanut, olive oil, or other acceptable carrier.

They are suspended in an aqueous carrier, for example, in an isotonic buffer solution at pH of about 5.6 to 7.4. These compositions can be sterilized by conventional sterilization techniques, or can be sterile filtered. The compositions can contain pharmaceutically acceptable auxiliary substances as required to approximate physiological conditions, such as pH buffering agents. Useful buffers include for example, sodium acetate/acetic acid buffers. A form of repository or “depot” slow release preparation can be used so that therapeutically effective amounts of the preparation are delivered into the bloodstream over many hours or days following transdermal injection or delivery. In addition, the compounds can be formulated in solid form and redissolved or suspended immediately prior to use. Lyophilized forms are also included.

Alternatively, certain molecules identified in accordance with the present invention can be administered orally. For oral administration, the compounds are formulated into conventional oral dosage forms such as capsules, tablets and tonics.

Systemic administration can also be by transmucosal or transdermal. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be, for example, through nasal sprays or using suppositories.

One route for administration of the compounds of the invention may be inhalation for intranasal and/or intrapulmonary delivery. For administration by inhalation, usually inhalable dry powder compositions or aerosol compositions are used, where the size of the particles or droplets is selected to ensure deposition of the active ingredient in the desired part of the respiratory tract, e.g. throat, upper respiratory tract or lungs. Inhalable compositions and devices for their administration are well known in the art. For example, devices for the delivery of aerosol medications for inspiration are known. One such device is a metered dose inhaler that delivers the same dosage of medication to the patient upon each actuation of the device. Metered dose inhalers typically include a canister containing a reservoir of medication and propellant under pressure and a fixed volume metered dose chamber. The canister is inserted into a receptacle in a body or base having a mouthpiece or nosepiece for delivering medication to the patient. The patient uses the device by manually pressing the canister into the body to close a filling valve and capture a metered dose of medication inside the chamber and to open a release valve which releases the captured, fixed volume of medication in the dose chamber to the atmosphere as an aerosol mist. Simultaneously, the patient inhales through the mouthpiece to entrain the mist into the airway. The patient then releases the canister so that the release valve closes and the filling valve opens to refill the dose chamber for the next administration of medication. See, for example, U.S. Pat. No. 4,896,832 and a product available from 3M Healthcare known as Aerosol Sheathed Actuator and Cap.

Another device is the breath actuated metered dose inhaler that operates to provide automatically a metered dose in response to the patient's inspiratory effort. One style of breath actuated device releases a dose when the inspiratory effort moves a mechanical lever to trigger the release valve. Another style releases the dose when the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978.

Devices also exist to deliver dry powdered drugs to the patient's airways (see, e.g. U.S. Pat. No. 4,527,769) and to deliver an aerosol by heating a solid aerosol precursor material (see, e.g. U.S. Pat. No. 4,922,901). These devices typically operate to deliver the drug during the early stages of the patient's inspiration by relying on the patient's inspiratory flow to draw the drug out of the reservoir into the airway or to actuate a heating element to vaporize the solid aerosol precursor.

Devices for controlling particle size of an aerosol are also known, see, for example, U.S. Pat. Nos. 4,790,305; 4,926,852; 4,677,975; and 3,658,059.

For topical administration, the compounds of the invention are formulated into ointments, salves, gels, or creams, as is generally known in the art.

If desired, solutions of the above compositions can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components can be mixed simply in a blender or other standard device to produce a concentrated mixture which can then be adjusted to the final concentration and viscosity by the addition of water or thickening agent and possibly a buffer to control pH or an additional solute to control tonicity.

The amounts of various compounds for use in the methods of the invention to be administered can be determined by standard procedures. Generally, a therapeutically effective amount is between about 100 mg/kg and 10⁻¹² mg/kg depending on the age and size of the patient, and the disease or disorder associated with the patient. Generally, it is an amount between about 0.05 and 50 mg/kg, or between about 1.0 and 10 mg/kg for the individual to be treated. The determination of the actual dose is well within the skill of an ordinary physician.

The compounds of the present invention may be administered in combination with one or more further therapeutic agents for the treatment of IgE-mediated allergic diseases or conditions.

Such further therapeutic agents include, without limitation, corticosteroids, beta-antagonists, theophylline, leukotriene inhibitors, allergen vaccination, and biologic response modifiers such as soluble recombinant human soluble IL-4 receptors (Immunogen), and therapies that target Toll-like receptors. (see, e.g. Barnes, The New England Journal of Medicine 341:2006-2008 (1999)). Thus the compounds of the present invention can be used to supplement traditional allergy therapy, such as corticosteroid therapy performed with inhaled or oral corticosteroids.

7. Articles of Manufacture

The invention also provides articles of manufacture comprising the vaccines herein. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The container may also be an inhalation device such as those discussed above. At least one active agent in the composition is a vaccine of the invention. The label or package insert indicates that the composition is used for treating the condition of choice, such as an allergic condition, e.g. asthma or any of the IgE-mediated allergies discussed above. The article of manufacture may further comprise a further container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Further details of the invention are illustrated by the following non-limiting Examples.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control.

### EXAMPLES

### Example 1

### Construction and Expression of IgE-Mediated Gene Delivery Vaccines

Human FcεRIα chain transgenic mice.

As mouse APCs (e.g., macrophages, monocytes and DCs) do not express FcεRI, the concept of IgE-mediated allergen gene delivery to FcεRI expressing DCs cannot be tested in conventional mice. Mice that carry a transgene for the human FcεRIα chain, e.g., hFcεRIα Tg mice (the mouse endogenous FcεRIα chain was also knocked out so as not to compete for signaling), critically show the human pattern of cell-specific expression of human Fc FcεRI ((Dombrowicz, D., et al., 1996. J. Immunol. 157:1645; Dombrowicz, D., et al., 1998. Immunity. 8:517). Thus, the h FcεRIα Tg mice express functional FcεRIα for human IgE not only on the mast cells, basophils, eosinophils, but also on APCs such as monocytes, Langerhans cells and DCs with the αβγ2 receptor complex on mast cells and basophils and αγ2 receptor complex on APCs (Dombrowicz, D., et al., 1996. J. Immunol. 157:1645; Dombrowicz, D., et al., 1998. Immunity. 8:517). As the hFcεRIα Tg mice lack the murine FcεRIα chain, they will produce but are not reactive via murine IgE. However, they produce IgG1 that can induce systemic and local allergic reactivity. This mouse strain was kindly provided by Dr. Jean-Pierre Kinet (Harvard Medical School, Boston, Mass.) and the mice have bred here for several years for other purposes. We have confirmed that the CD11c DCs from the hFcεRIα Tg mice express human FcεRIα on the cell surface, as determined by an anti-human FcεRIα antibody from eBioscience, San Diego, Calif. 92121, USA.

### Human IgE

We have expressed and purified large amounts of recombinant human IgE and IgE isoforms (Lyczak, J., B., et al., 1996. J. Biol. Chem. 271:3428). A large quantity of IgE was purified from IgE myeloma patient PS's serum (provided by Drs. R. McIntyre and K. Ishizaka).

### Fascin Promoter Vectors.

The mouse Fascin promoter controlled expression vectors were constructed by cloning the 2.6 Kb mouse Fascin promoter (Sudowe, S., L et al., 2006. J Allergy Clin Immunol. 117:196-203) by PCR. The CMV promoter in the pCMV-EGFP vector or in pcDNA3.1-Fel d1 was replaced by conventional cloning methods with the isolated Fascein promoter.

### Fel d1 Gene.

An engineered gene that expresses both chains of the Fel d1 antigen, the dominant allergen from cats, has been obtained from Drs. Paul Guyre and Amanda Sun (Dartmouth College) (Vailes, L. D., et al., 2002). J. Allergy Clin. Immunol. 110:757).

### Construction of the Expression Vector Specifically Activated in DCs.

Targeting FcεRI bearing cells will be accomplished by use of human IgE plus DNA polyplexes. To efficiently and selectively express the transferred gene in FcεRI bearing DCs, we will use an actin-bundling protein Fascin promoter controlled green fluorescence protein (GFP) expression construct as the model transferred gene construct. As maturing and mature DCs and follicular DCs are the only hematopoietic cells that express Fascin (Ross, R., et al., 1998. J Immunol. 160:3776; Ross, R., et al., 2000. J Invest Dermatol. 115:658; Mosialos, G., et al., 1996. Am J. Pathol. 148:593; Mosialos, G., et al., 1994. J. Virol. 68:7320; Pinkus, G. S., et al., 1997. Am J Pathol. 150:543; Bros, M., et al., 2003. J Immunol. 171: 1825; Ross, R., et al., 2003. Gene Ther. 10:1035), the Fascin promoter should ensure selective DC-specific expression of the transgene. This will be compared to transfer of a CMV immediate early promoter (pCMV) construct that is expected to show promiscuous cell expression. The pCMV is expected to drive GFP expression in all types of cells mediated by IgE-FcεRI dependent gene transfer, including APCs, mast cells and basophils, whereas the Fascin promoter only functions in DC but not mast cells and/or basophils and therefore should provide cell type-specific gene expression fashion in DCs (Ross, R., et al., 1998. J Immunol 160:3776; Ross, R., et al., 2000. J Invest Dermatol. 115:658; Mosialos, G., et al., 1996. Am J Pathol. 148:593; Mosialos, G., et al., 1994. J. Virol. 68:7320; Pinkus, G. S., et al., 1997. Am J Pathol. 150:543; Bros, M., et al., 2003. J Immunol. 171: 1825; Ross, R., et al., 2003. Gene Ther. 10:1035).

We will construct a model CMV immediate early promoter (pCMV) controlled and a Fascin promoter controlled Green Fluorescence Protein (GFP) plasmid that will be used to determine the efficiency of the targeted gene delivery and expression. This will provide evidence of cell type-specific gene expression.

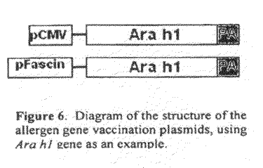

Specific plasmids containing a major allergen gene cDNA [peanut allergen Ara h1 and Ara h2, kindly provided by Drs. W. Burks and G. Bannon, formerly from the Univ. of Arkansas) (Shin D S et al. J Biol Chem. 73:13753, 1998), egg allergen ovomucoid (Gal d1) or milk allergen acasein (kindly provided by H. Sampson of Mt. Sinai Medical Center)] under the transcriptional control of the mouse DC-specific Fascin promoter will be constructed for use as the allergen gene vaccines for peanut, egg or milk allergy immunotherapy (FIG. 6).

We have constructed a plasmid containing the peanut allergen Ara h1 under the transcriptional control of the CMV immediate early promoter (pCMV). An Ara h1 cDNA containing plasmid (pbluescript-Ara h1, provided by Dr. A. W. Burks and G. Bannon, formerly from the Univ. of Arkansas) was digested with Not I and Apa I to release a 2.0 Kb fragment, and insert this fragment into pcDNA3.1 vector in Not I-Apa I sites.

The plasmids used will be on the pcDNA3.1 background, and the empty vectors (without the corresponding gene sequence) will be included as mock controls, unless specified. All the plasmid constructs will be prepared with an endotoxin-free plasmid preparation kit, and the residual amount of endotoxin level will be determined by Limulus assay.

It has been reported that a lead sequence that directs expressed protein to be secreted outside of cells may facilitate induction of cellular immune responses to DNA vaccination (Jiang C et al., Infect Immunol 70:3539, 2002). We chose not to include a leader sequence for our allergen vaccination purposes since the lead sequence in the plasmid was found to significantly increase antigen-specific IgE production for some allergen DNA vaccination (Tan L K et al., Vaccine. 24:5762, 2006). Furthermore, we are not particularly interested in having the allergen protein or its fragments secreted, by the APCs. Indeed, any secreted expressed antigen (hence allergen) would have the potential to trigger at least a local allergic reaction.

### Preparation of IgE-PLL

Poly-L-lysine (PLL), a type of polycation reagent, is widely used for protein-DNA vector complex formation for gene delivery (Cristiano R. J. 1998. Front Biosci. 3:d1161), as this method utilizes non-damaging ionic charges rather than chemical covalent crosslink between the protein and the DNA expression vector (Cristiano R. J. 1998. Front Biosci. 3:d 1161). PLL does not possess antigenicity, therefore, PLL complexed DNA can be repeatedly administrated.

PLL has been chemically crosslinked to IgE (see below). The IgE-PLL complex could be mixed with a CMV- or Fascin-promoter controlled GFP expression vector to conically form IgE-PLL:DNA vector polyplexes for IgE-mediated gene delivery.

There are multiple methods available for the cross-linking of PLL to proteins (Cristiano R. J. 1998. Front Biosci. 3:d1161), however, their relative efficiencies for targeting a given gene to a specific type of cell have not been compared. We will compare three methods for efficiency of gene delivery and expression. We will use the ethyl-3(3-dimethylaminopropyl) carbodiimide (EDC) coupling method (Wu, G. Y. & Wu, C. H. 1987. J Biol Chem. 262: 4429), 3-(2-pyridyldithio) propionic acid N-hydroxysuccinimide ester (SPDP) method (Wagner, E, et al., 1990. Proc Natl Acad Sci USA. 87:341049) and biotin-streptavidin method (Cotten, M., et al., 1992. Proc Natl Acad Sci USA. 89:6094) for the coupling of IgE to PLL.

IgE-PLL crosslinking has been done with a directional crosslinking protocol (Wu, G. Y. & Wu, C. H. 1987. J Biol Chem. 262: 4429). The intention was that IgE will only be cross-linked to PLL but not to itself. The possibility of two or more IgE simultaneously cross-linked to one PLL (which would form multiple IgE containing complexes) can be limited by adjusting the molar ratio of IgE to PLL in a 1:1 ratio. The chemical cross-linking method to prepare the IgE-PLL complex for gene delivery would have potential side-effects of unwanted IgE crosslinking leading to non-monomeric IgE molecules that could potentially trigger an allergic reaction. In addition, the size and degree of the crosslinked IgE-PLL complexes are difficulty to control by this method due to the nature of chemical reaction, therefore the products from batch to batch could be significantly different.

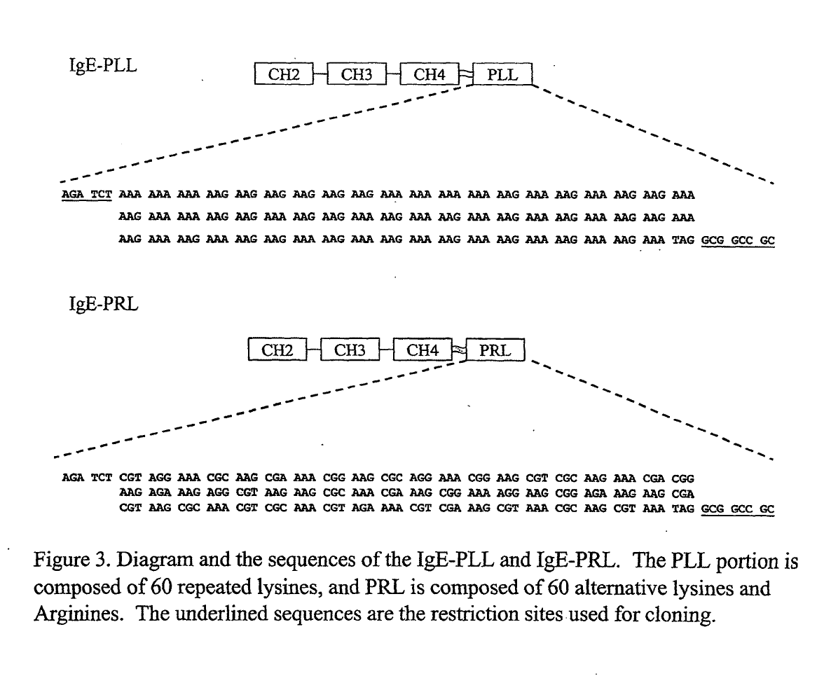

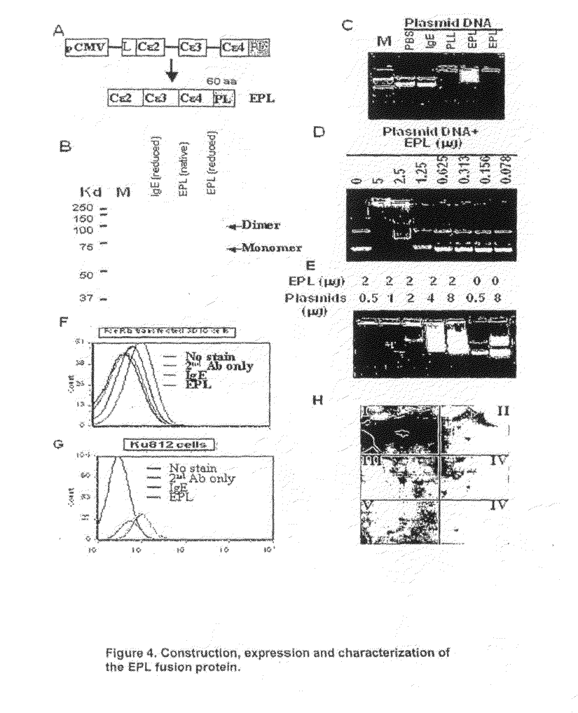

In order to overcome these problems, we used a recombinant DNA technique to produce IgE-PLL by construction and expression of the fusion gene of the human IgE heavy chain (CH2-CH3-CH4) linked with 180 by synthesized DNA coding for 60 repeated lysines (for IgE-PLL). (Diagrammed in FIG. 4A). This construct is called Fc Epsilon-PolyLysine protein or “EPL”. We also constructed an IgE-PRL DNA construct using recombinant DNA techniques to link the human IgE heavy chain (CH2-CH3-CH4) with 180 by synthesized DNA coding for alternately repeated lysines and arginines. See FIG. 3 for a diagram of the construction. In FIG. 3, the restriction sites for cloning purpose are underlined. This approach ensures that each IgE molecule is uniformly associated with PLL or PRL so that the potential IgE crosslink would not occur, and the quality of the product would be same for all the experiments performed at different time.

Prior to use, any residual amounts of multimeric IgE-PLL complexes will be removed by FPLC (Cristiano R. J. 1998. Front Biosci. 3:d1161).

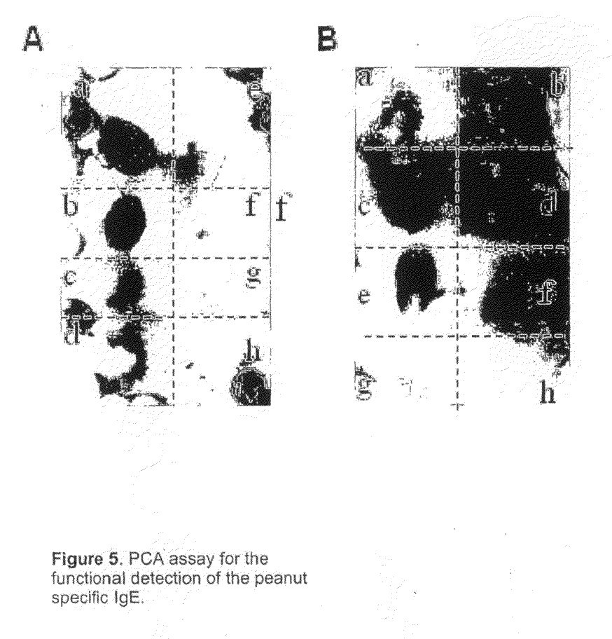

EPL protein expressed in mammalian (NSO) cells was affinity purified on anti-IgE columns and eluted protein analyzed by Coomassie blue staining and Western blotting. Coomassie blue staining showed that the expressed EPL fusion protein under native (non-reduced) conditions primarily migrated with a molecular mass of 120 Kd, but under reducing conditions was present primarily as a 60 Kd mass; this indicates that EPL mainly assembled as the expected dimer (FIG. 5B). This expression of EPL as a dimer is critically important as the two-epsilon chain dimer is required to achieve the particularly high affinity of Fcε for a single FcεRI (Garman S C, Wurzburg B A, Tarchevskaya S S, Kinet J P and Jardetzky T S. “Structure of the Fc fragment of human IgE bound to its high-affinity receptor FcεRIα.” Nature 406:259, 2000).

### Fusion Protein Expression

EPL plasmid was transfected by electroporation into 2−4×10⁷ Ns0/1 myeloma cells. The cells, including 2×10⁶ cells for a no DNA control, were spun at 1000 rpm for 5 min, resuspended in 0.5 ml cold PBS, and placed in a 0.4 cm electroporation cuvette (BioRad, Hercules, Calif.). 50 μl linearized plasmid DNA in PBS was added to the cuvette and incubated on ice for 10 min. The cells were pulsed with 200V, 960 μF and then set on ice for 10 min. Cells were washed in 10 ml Iscoves' Modified Dulbecco Media (IMDM, Irvine Scientific, Santa Ana, Calif.)+5% Supplemented Bovine Calf Serum (CS, Hyclone, Logan, Utah) and plated at 2×10⁶ cells/plate in IMDM+10% calf serum. Two days later, the cells were fed with selective media containing IMDM+10% CS+1 mg/ml geneticin (Invitrogen). Selective media was replenished after three days. Wells that contained colonies were tested by ELISA. Protein producing cells were grown in roller bottles and the protein was purified on an anti-IgE affinity column (Sigma Aldrich, St. Louis, Mo.) by acid elution using citric acid pH 4.5 and glycine pH 2.5. 1 ml protein fractions were neutralized with 2 M Tris, pH 8.0 and then dialyzed against PBS.

### SDS-PAGE:

Purified protein were denatured by boiling in 1× sample buffer (25 mM Tris, pH6.7, 2% SDS, 10% glycerol, 0.008% bromophenol blue) for 2 min and the non-reduced samples separated SDS-PAGE at 150 mAmp. The denatured samples were also reduced by boiling with 1%β mercaptoethanol for 2 min and separated by SDS-PAGE.

### Flow Cytometry:

Binding of the EPL fusion protein to FcεRI was assessed by flow cytometry on 3D10 and Ku812. Cells were grown in Iscove's Modified Dulbecco's Media (IMDM, Irvine Scientific, Santa Ana, Calif.)+10% Fetal Calf Serum. For each sample, 10⁶ cells were washed in 1 ml PBS, pH 7.4, spun at 2000 rpm for 5 min and the supernatant was removed. The cells were resuspended in 100 μl IMDM+10% FCS with or without EPL and IgE proteins at several concentrations and incubated at 4° C. for 1 hour. The cells were washed twice with 1 ml PBS and then incubated at 37° C. with 100 μl 10 μg/ml FITC labeled goat anti-human epsilon chain (Sigma) for 30 min at 4° C. Cells were washed 3 times in 1 ml PBS and resuspended in 500 μl 2% paraformaldhyde in PBS. Samples were analyzed on a FACScan flow cytometer (Becton Dickinson Immunocytometry Systems, San Jose, Calif.), gating out dead cells and debris.

### Modification of the IgE-PLL to Enhance Cellular Uptake and Expression.

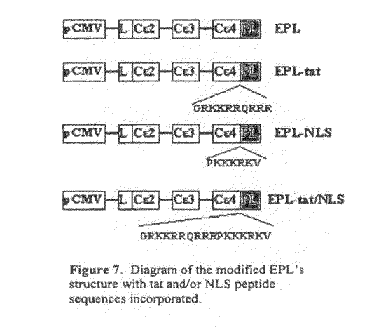

The experiments utilize a specific targeting (IgE-FcεRII) and expression (Fascin promoter) mechanisms as the basic key elements in our allergen vaccine approach. Modifications could be made to enhance the penetration of the plasmid DNA into the cells and/or direct the plasmid DNA to the nucleus for longer-term expression of the DNA vaccine. Such modifications include incorporation of a HIV tat peptide sequence (GRKKRRQRRR) and/or a nuclear localization signal (NLS) peptide (PKKKRKV) into the backbone of EPL (FIG. 7). This HIV tat peptide sequence has been shown to significantly enhance the transportation of a variety of molecules including large drugs and DNA into cytoplasm (Brooks H et al., Adv Drug Deli Rev 57:559, 2005), while the NLS is capable of directing the plasmid into the nucleus for more efficient expression of the targeted gene, as some plasmids reached in nucleus would integrate into the host chromosome for long-term expression of the allergen in APC (Talsma S S et al., J Control Release 112:271, 2006). With these modifications, the efficiency of the IgE-mediated allergen gene vaccination is expected to be significantly enhanced.

### Preparation of IgE-PLL:DNA Polyplexes

The polyplexes of IgE-PLL and the pCMV-ara h1 plasmid were assembled simply by mixing the appropriate amount of EPL and plasmid DNA in PBS for 30 min at 25° C. prior to injection. Polyplexes of IgE-PLL and other plasmids could be assembled using the same procedure.

We showed that the expressed EPL, as well as PLL, but not IgE alone, were capable of binding plasmid DNA (pCMV-GFP) in a gel retardation analysis (FIG. 4C), in both an EPL protein concentration-dependent fashion (FIG. 4D) and in a plasmid DNA concentration-dependent fashion (FIG. 4E). The EPL-DNA polyplex was shown by FACS analysis to bind to FcεRI expressed on 3D10 cells (CHO cells that express the human FcεRIα chain) (FIG. 4F) and Ku812 cells, a human mast cell-like line that expresses the entire FcεRI receptor complex (FIG. 4G). In a passive cutaneous anaphylaxis assay with a human FcεRIα transgenic mouse, we demonstrated that the assembled EPL:DNA polyplex did not trigger local allergic skin reaction (FIG. 4H); this indicates that the EPL:DNA polyplex did not crosslink the FCC or trigger mast cell degranulation. Passive cutaneous anaphylaxis (PCA) was performed as following. The mice that have been genetically engineered to carry the human receptor for human allergic antibody and thus they can react to such human allergic antibodies was used for PCA. These experiments test whether chimeric human Ig proteins can block the classic passive cutaneous anaphylaxis reaction in the skin of these animals. Mice were given 4 to 6 injections in the back skin with 50 ul of volume for each site. These injections will contain human allergic antibodies that can make those areas “allergic”. Some of the spots will also have been administered chimeric proteins that are designed to block the development of allergic reactions at those same spots. Four to six hours later the mice will then be given intravenously by tail vein injection, a corresponding allergen that reacts with the human allergic antibody along with 1% of Evan's blue dye in 200 μl volume. Twenty to 30 minutes later, the animals will be euthanized and the size of the reaction (bluing) at each site evaluated.

### Testing IgE-DNA Polyplex Uptake and Expression In Vitro.

The prepared IgE-PLL:GFP polyplexes (pCMV controlled) will be tested for their efficiency in IgE-mediated vector DNA transfer by evaluating GFP expression in a human APC-like cell line, U937, that expresses the normal APC αγ2 FcεRI complex or in the human mast-like cell line LAD2. The LAD2 cell line expresses functional FcεRI (Jensen B M, et al., 2005. Int Arch Allergy Immunol. 137:9351) and is able to internalize the FcεRI binding IgE. Controls for gene transfer and expression efficiency will include PLL:GFP, IgE plus GFP vector, and GFP vector alone by culturing the cells with the DNA polyplex and their corresponding controls. To test the fascin promoter driven GFP expression in DCs, we will use CD11c positively selected DCs by MACS cell sorting (Williamson E, et al., 2002 J. Immunol. 169: 3606) from hFcεRIα⁺Tg mice for IgE-mediated GFP vector transfer and expression. DCs from FcεRIα negative littermates will serve as controls. The cells will be cultured with the various polyplexes for 2 to 5 days and the resulting GFP expression will be assessed by fluorescence microscopy or by flow cytometry. The polyplex preparation method generating the highest GFP expression level in the in vitro culture system will be used to prepare the EPL:GFP vector polyplexes for in vivo gene delivery testing.

One important issue for the delivered gene expression is that at least a small portion of the uptake DNA vector should be released from the endosomes into either the cytosol or nucleus compartments before the endosomes are fused with lysosomes, where DNA vectors are subject to degradation. Since both IgE and FcεRI receptors are recycled to cell surfaces in the process of the FcεRI-mediated endocytosis (Furuichi K, et al., 1986. J Immunol. 136:1015: Borkowski, T. A., et al., 2001. J Immunol. 167: 1290), this suggests that FcεRII-bound DNA vectors via FcεRI-mediated endocytosis are not likely subjected to lysosomal degradation. Therefore, there is every likelihood that adequate amounts of the IgE-mediated allergen DNA uptake will be released and expressed in the DCs. If we do not see adequate expression, it may be because PLL is a linear polycation reagent and, as such, it may not efficiently mediate endosomal membrane disruption and DNA release before fusion to lysosomes with subsequent DNA destruction. If this becomes an issue, we can use the branched chain polycation reagent polyethylenimine (PEI) in place of PLL. PEI is highly branched and efficiently disrupts the endosomal membrane prior to lysosome fusion (Boussif, 0., et al., 1995. Proc Natl Acad Sci US A. 92:7297). This leads to increased release of DNA vector into cytosol where it can be expressed, and it has been shown that gene expression is enhanced by 4-5 orders of magnitude in this fashion (Cristiano R. J. 1998. Front Biosci. 3:d1161; Boussif, O., et al., 1995. Proc Nail Acad Sci USA. 92:7297).

### Establishment of a PCA Assay to Functionally Detect Human IgE-Driven Peanut Allergic Reactions.

Anti-peanut IgE antibody is responsible for the systemic anaphylaxis of peanut allergy in humans, whereas both IgE and IgG1 are important for the peanut allergy in the mouse model. We have previously established a PCA assay to functionally assess in vivo allergic responses in the hFcγRIα Tg mouse model but not for peanut allergy (Zhu D et al., Nat Med 8:518, 2002. Kepley C L, Zhang K, Zhu D, and Saxon A. Clin. Immunol 108: 89-94, 2003). We have now established a similar PCA assay for peanut allergic responses in the hFcεRIαTg mice. Serum from peanut allergic patients (kindly provided by Dr. Hugh Sampson) was serially diluted and injected into the back skin of FcεRIα Tg mice. Twenty-four hours later the mice were challenged with purified Ara h1 antigen. As shown in FIG. 5A, there was a dose dependent PCA reaction to the peanut allergic patient's serum (from 5a to 5f), but no reaction to the serum from a healthy donor (5g) or to saline (5h). We have used this assay to screen several batches of the peanut allergic patient's sera (FIG. 5B) from a commercial source (Plasma Lab, WA). The strongly positive sera (samples 5b and 5c) were purchased in bulk for the purification of the peanut allergen specific IgE. These results show that: 1) the FcεRIα Tg mice express skin reactivity to IgE to peanut patients' serum in vivo challenge (albeit it is passively transferred human IgE), and 2) they demonstrate our ability to do allergen specific graded skin testing in the FcεRIα Tg mice that we actively sensitized.

### Testing Model IgE-DNA Polyplex Expression In Vivo.

To assess the efficiency of the IgE-mediated gene delivery vectors in vivo, we will inject EPL:pFascin-GFP polyplex into FcεRIα+tg mice. Varying doses of the polyplex will be administrated intravenously (i.v.) by tail vein injection. We purposely will use the i.v. route as that will disseminate the vaccine to all essential tissues for later examination. We will examine the anatomic localization of the DCs that express GFP in histological sections from the lymphoid organs (spleen, lymph nodes, and gut-associated lymphoid tissues, Peyer's patch and thymus) of the FcεRIα+tg mice bu immunoflourescence at Days 3-5. These results will provide direct in vivo testing of the efficiency and localization of the IgE-mediated gene transfer. A PLL:pFascin-GFP combination without the IgE will serve as an additional control for the efficiency of the DNA delivery and expression in DCs. We will also test intradermal (i.d.) administration as it is readily performed in humans, is commonly used in gene vaccination approaches, and it will allow us to examine the local tissue for gene expression. The local skin and lymph nodes will be examined for GFP expression 2-5 days after DNA vaccination. The FcεRIα− tg mice in which IgE focusing does not occur will serve as the background controls.

### Testing Expression of IgE-Fel d1 Gene Polyplexes In Vivo.

We will repeat the expression experiments discussed above in h FcεRIα Tg mice but use an IgE-PLL:Fel d1 gene expression system. We will use immunohistochemical methods to detect the presence and localization of expressed Fel d1. Dose and timing experiments will be undertaken to establish the parameters that give optimal Fel d1 expression in DCs.

Testing Expression of EPL:Allergen Gene (Ara h1, Ara h2. Ara h3 and Gal d1) Polyplexes In Vivo.

We will repeat the in vivo expression experiments discussed above in hFcεRIαtg, using EPL:allergen gene (Fascin promoter controlled) polyplexes. We will use immunohisto-chemical methods to detect the presence and localization of the expressed Ara h and Gal d1 proteins with available allergen specific monoclonal antibodies. These experiments are to verify that we are achieving in vivo expression of the vectors and that we have the refined technologies to optimally detect the allergen vaccines' expression in vivo. Again, the hFcεRIα− tg littermates will serve as negative controls for comparison.

### Example 2

### Determination of the Effects of IgE-Mediated Feld1 Gene Vaccination on the Induction of Fel d1 Allergic Responses in h FcεRIα Tg Mice

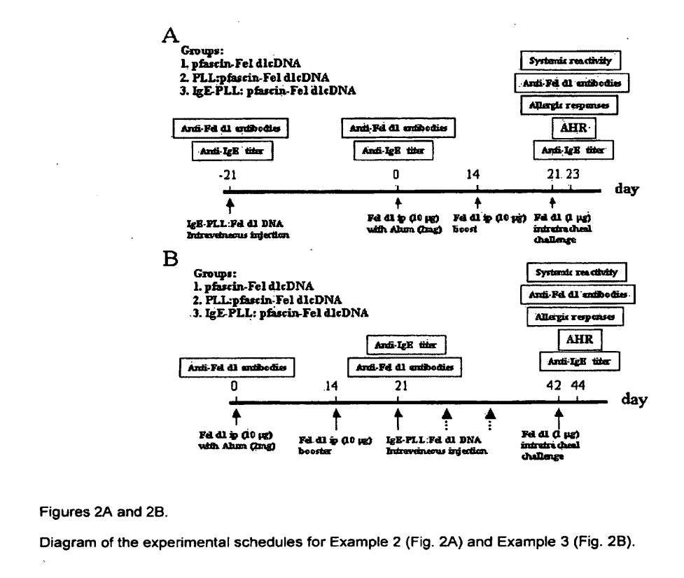

To determine the effects of the IgE-mediated Fel d1 gene vaccination for preventing Fel d1-induced allergic responses, the experiments will be conducted as diagrammed in FIG. 2A. Groups (8 mice per group) of hFcεRIα Tg mice will be vaccinated on Day −21 with (a) the IgE-PLL:Fel d1 containing DNA vector polyplex, (b) a control PLL:Fel d1 containing DNA vector combination, and (c) Fel d1 containing DNA vector only. Three weeks later, (Day 0) the mice will be sensitized with 10 μg Fel d1 intraperitoneally (i.p) in alum and then boosted on Day 14 with Fel d1 antigen using one of our established protocols known to induce systemic allergic responses and airway hypersensitivity (Zhu C., et al., 2005. Nat. Med. 11:446; Terada, T., et al., 2006. Clin Immunol. 120:45, 2006). The animals will then be challenged at Day 21 with intratracheal Fel d1 (1 μg), and the designed experimental parameters, as shown in Table 1, will be examined two days post intratracheal Fel d1 challenge (Day 23)(FIG. 2). Physiologic read-outs will consist of changes in core body temperature to measure systemic reactivity reflecting the basophil degranulations (Zhu C., et al., 2005. Nat. Med. 11:446; Terada, T., et al., 2006. Clin Immunol. 120:45, 2006). To determine the effects of IgE-mediated Fel d1 gene vaccination on airway hyperresponsiveness (AHR), the airway resistance to methacholine challenge will be accessed by using a computer-controlled small animal ventilation-pulse oscillometry system (Flexi-vent®) (Zhu C., et al., 2005. Nat. Med. 11:446; Terada, T., et al., 2006. Clin Immunol. 120:45, 2006).

After sacrifice, we will tie off the lungs individually and obtain BAL fluid from one lung to measure the levels of the key regulatory and polarized cytokines and chemokines as indicated in the Table 2, and the cellular compositions of the BAL fluid to evaluate the status of airway allergic responses and lung inflammation. Cellular composition and cytokine producing profiles from the cells infiltrated in the lung will be also analyzed by digestion of the lung with collagenase D; the resulting cells will be analyzed for the composition of T cell subpopulations (CD4/CD8 ratio), Th1 or Th2 types by cellular staining of IFN-γ and IL-4, respectively, with flow cytometry. The non-lavaged lung will be assessed histologically for changes of allergic reactivity. Spleen cells will be prepared and tested for spontaneous and Fel d1 induced production of key cytokines (IL4, IL-5 and IFN-γ) that represent memory T cell responses and Th1/Th2 response profiles.

Fel d1-specific IgE, total IgG, IgG1, IgG2a, IgG2b, IgG3, IgA and IgM antibodies will be assayed using conventional ELISAs. These will be measured prior to vaccination (Day −21), at the time of the immunization (Day 0), as well as at the end of the experiments (Day 23), since it is possible that the vaccination will lead to a response just prior to the intratracheal challenge (Day 21). The statistical significance among the experimental parameters described above among the experimental groups will be compared. Anti-human IgE responses will be checked on Day −21, Day 0 and Day 23 to determine the level of anti-human IgE response. This set of experiments will allow us to determine whether allergen gene vaccination is able to alter a subsequently induced Fel d1-specific allergic response as a model for prophylactic intervention and to determine the effects of the vaccination on the Th1/Th2 balance therein, as well as to determine the potential mechanisms by which the IgE-mediated allergen gene vaccination exerts the immunotherapy effects on allergen-specific allergic systemic responses and airway hyper-responsiveness.

Route of vaccination: We will give the vaccination i.v. This is predicted to be the most efficient route of delivery. Experiments could be done later with intramuscular (i.m.) or subcutaneous (s.q.) injections. Notably, if i.v. administration is effective, this route might even be used in humans as few injections are predicted to be necessary and thus the i.v. route might be practical.

Dose: The dose used will give optimal gene expression in DCs.

Timing for vaccination: As in our experimental model for prophylactic allergen gene vaccination, the vaccinations are generally done 21-35 days prior to allergen sensitization (e.g., between Day −35 and −21 in the diagrammed schedule).

Number of vaccinations: For several reasons, we plan to use one vaccination and modify the dose and timing prior to giving multiple gene vaccinations in this protocol. As discussed, we feel our IgE-focused gene delivery will be much more effective than previous methods that often require more than one vaccination. Furthermore, as repeat gene vaccination is generally done one week or more apart, it is likely the mice will react to repeated administration of the human IgE protein and thereby this would complicate the interpretation of the results. There are alternatives to overcome this possibility of immunogenicity.

TABLE 2
Endpoint Assessments in Mice

| Functional endpoint | Assessments |
|---|---|
| AirwayHyper | Resistance by pulse oscillometry |
| reactivity | |
| Lung inflammation | Lung histology, BAL immune cellular |
| | composition |
| Systemic reactivity | Core body temperature |
| Antibody response | Fel d1 specific IgE, IgG, IgG1, 2a, 2b, IgA, |
| | IgM |
| Cellular response | Key cytokines and chemokines in BAL fluid |
| | (IL-4, 5, 13, 10, 12, INFγ, TGF-β, TNFα, |
| | eotaxin, and RANTES. |
| | Spontaneous and antigen induced cytokine |
| | response profile of spleen cells |
| Response to human | Antibody to human IgE |
| IgE | |

### Example 3

### The Effects of IgE-Mediated Fel d1 Gene Vaccination on Established Allergic Responses to Fel d1 in hFcεRIα Tg Mice

To determine if our proposed IgE-mediated Fel d1 gene vaccine can alter an already established allergic response, Fel d1-induced allergic responses will be established prior to allergen DNA vaccination and the allergic animals will be treated according to the schedule outlined in FIG. 2B. The hFcεRIα Tg mice will be sensitized by i.p. injection with Fel d1 plus alum at Day 0, followed by an i.p. booster of Fel d1 alone at Day 14. On Day 21, the mice will receive an i.v. treatment with the IgE-PLL:Fel d1 gene expression vector, with PLL:Fel d1 gene expression vector, and with Fel d1 gene expression vector alone as the experimental control. Twenty-one days later (Day 42), the mice will be challenged intratracheally with Fel d1 to induce a systemic response and airway hypersensitivity, using the protocol previously established (Zhu C., et al., 2005. A Novel Fcγ-Fel d1 Protein for Cat-induced Allergy. Nat. Med. 11:446; Terada, T., et al., 2006. A chimeric human-cat Fcγ-Fel d1 fusion protein inhibits systemic and pulmonary allergic reactivity to intratracheal challenge in mice sensitized to the major cat allergen Fel d1. Clin Immunol. 2006 July, 120(1):45-56). The designed experimental parameters, as shown in the Table 2, will be examined at Day 44. Fel d1-specific IgE, total IgG, IgG1, IgG2a, IgG2b, IgA and IgM antibodies will be measured prior to sensitization (Day 0), at the time of the gene vaccination (Day 21) and just prior to the intratracheal challenge (Day 42). Anti-human IgE responses will be checked at Days 21 and 44, as well as to examine whether anti-human IgE response is mounted. The statistical significance among the experimental parameters described above among the experimental groups will be determined and compared. This set of experiments will determine the relative efficiencies of IgE-mediated, compared to non-IgE mediated, allergen gene vaccination to physiologically and immunologically alter an established Fel d1-specific allergic response involving the airway as a model for intervention in ongoing allergic asthma, and the potential mechanism for the allergen gene vaccination immunotherapy.

Design issues: In addition, the issue of repeated vaccinations may well become important in the experiments designed to treat established allergic disease. Thus, if the single vaccination is unsuccessful at Day 21, in addition to modifying the dose we would propose that additional booster vaccinations at Day 28 and Day 35 (indicated in FIG. 2B with dotted arrows) would be undertaken to enhance the efficacy of the allergen gene vaccination. As human IgE is a foreign protein to mice, the development of murine antibody against human IgE following first administration is likely to occur and possibly to interfere with the efficacy of the subsequent vaccinations, e.g. by blocking hFcεRIα binding and by altering clearance of the DNA vaccine. Thus, if we employ the protocol with more than one time gene vaccination, we will use one of two alternative approaches to circumvent this potential pitfall. We can induce neonatal tolerance to human IgE in the hFcεRIα mice simply by giving i.p. injection of human IgE to the new-born mice at Day 1 and Day 3, a protocol for efficient neonatal tolerance induction (Wekerle T., and Sykes, M. 2001. Mixed chimerism and transplantation torerance. Annual Review of Medicine. 52: 35358). The resulting human IgE-tolerant mice could be used for the experiments that employ more than one IgE-mediated Fel d1 gene vaccination. Alternatively, we may use hFcεRIα Tg mice that have the human IgE knocked-in as human IgE is then “self” to these animals. Potential antibodies against human IgE interfering with IgE-mediated gene delivery is a problem specific to murine experiments; it will not occur in humans where human IgE is “self”.

### Example 4

### The Immunomodulatory and Therapeutic Affects of IgE-Mediated Allergen Gene Vaccination In Vivo

Human IgE Knockin-hFcεRIα tg Mice (hIgE+-hFcεRIα⁺ tg Mice), the Ideal Mouse System to Test IgE-Mediated Gene Vaccination as Therapy for Allergic Disease:

To target allergen genes to APCs through the IgE-hFcεRI interaction, we will employ mice expressing the human hFcεRIα. However, these mice have two shortcomings when it comes to full in vivo testing of the human IgE protein-allergen gene polyplexes. First, the Fce part of EPL serves as an antigen in the animals so that repeated vaccinations will be problematic due to the murine anti-human epsilon response. A second difficulty with the hFcεRIα μg mice is that any murine IgE produced as part of the sensitization protocol will fail to function in vivo as the murine hFcεRIα has been knocked out and murine IgE binds poorly to the human hFcεRIα. We will overcome both issues by employing hFcεRIα tg mice modified by having the human Ig epsilon gene knocked-in in place of the mouse endogenous epsilon gene, e.g. hIgE⁺-hFcεRIα⁺ tg mice. In hIgE⁺-hFcεRIα⁺ tg mice, the sensitization will drive human IgE production as well as murine IgG and other non-IgE isotypes. The human IgE will be functional via human IgE-hFcεRIα interactions in these mice. Repeated administration of EPL as part of the gene vaccination should not induce immune responses against the human epsilon portion of the EPL for just as with humans, these animals express human epsilon as “self”. An additional benefit is that it is likely that the human IgE will enhance the level of expression of the FcεRI, as this is a well-described positive feedback effect (Kinet J P. Annu Rev Immunol 17:931, 1999). These animals have been produced and will be supplied by Dr. J-P. Kinet.

It has been demonstrated that allergen gene vaccination generally induces a Th1 type, instead of an allergen protein driven Th2 type, responses due to the CpG nucleotide sequence presented in the plasmid backbone that functions as adjuvant for Th1 dominant response (Roman M et al., Nat Med 3:849, 1997; Chatel J M et al., Allergy 58:641, 2003). We expect that the strategy of targeting of allergen gene to DC would induce an even stronger Th1-dominate immune response than that of the conventional allergen gene vaccination. It is anticipated that the vaccine will be more active than placebo in causing recognition of the allergen and that it will be distinct from control vaccines (e.g. naked DNA vaccine at the same dose) in inducing an allergen specific response.

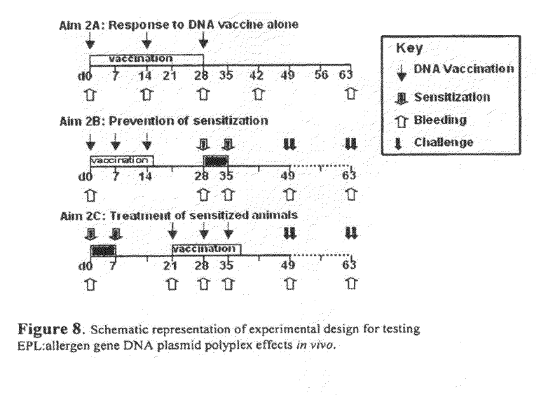

The immune response profiles induced by IgE-mediated allergen gene vaccination for Aha h1 and Gal d1 will be determined. (See FIG. 8 Example 3)

The hIgE⁺-hFcεRIα⁺ tg mice (4-6 week old) were be i.d. vaccinated with the EPL:pCMV-controlled arah1 gene polyplexes. Initially, we gave a single vaccination. A second and third vaccination will be given at two-week intervals.

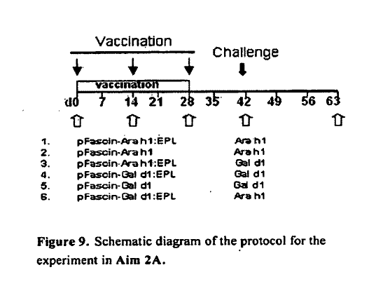

The hIgE⁺-hFcεRIα⁺ tg mice (4-6 week old) will be i.d. vaccinated with the EPL:pFascin-controlled allergen gene polyplexes (FIG. 9, groups 1 and 4, respectively). Initially, we will give a single vaccination and follow the response and then a second and third vaccination will be given at two-week intervals. While gene vaccinations are often weekly or more frequently, we specifically chose two-week intervals to allow us to test the response to each vaccination before giving the next. The “naked DNA” vaccination (group 2 and 5 for Ara h1 and Gal d1, respectively) will serve as vaccine controls. The EPL:Ara h1 plasmid vaccination (group 3) will serve as allergen specific control and background plasmid control for Gal d1 gene vaccination, and vice versa (Group 6), making the empty plasmid control group unnecessary. We will use the Ara h1 and Gal d1 vaccines at 10 μg of plasmid DNA per mouse as prototypes in these experiments although this may be modified. The experiment will be set up so that all mice begin the sequence together; mice will be sacrificed at days 0, 14, 28 and 42, representing baseline, first, second and third vaccination effects, respectively. Animals not sacrificed at a given time point will provide serum so that we have a continuous set of samples on each animal group up to termination.

### Antibody Response:

Serum will be collected (days 0, 14, 28, 42 and 63) for the measurement of Ara h1-, or Gal d1-specific human IgE and Ara h1-, or Gal d1-specific murine total IgG, IgG1, IgG2a, IgG2b, IgG3, IgA and IgM antibodies by ELISA. The antibody titers from day 0 serum will be taken as background controls, day 14 would reflect the primary response, and the day 28 and 42 levels would be secondary antibody responses in boosted animals. The antibody response at day 63 would be also monitored to determine whether the developed antibody response would fade in a relatively longer term. These experiments will define each allergen vaccine's ability to induce a humoral response and simultaneously quantify its level and isotype profile. Murine anti-human epsilon response will be checked by our standard ELISA to be sure that anti-human epsilon response is absent in the hIgE+-hFcεRIα tg mice as predicted because appearance of mouse anti human-epsilon might complicate the interpretation of the outcomes. A stronger allergen specific IgG2a (using IgG2a/IgG1 ratio as the assessment), but not IgE, response will be induced in the groups using EPL:DNA polyplex compared with that of naked DNA. IgG1 response will be monitored. If we unexpectedly observe a vaccination-induced strong IgE and/or IgG1 response, as occurred in the C3H/HeJ strain of mouse vaccinated with Ara h2 (23), we will determine whether the vaccination acts as a sensitization process by challenging the vaccinated mice with Ara h1 or Gal d1 protein as diagrammed in FIG. 9, and the systemic anaphylactic reaction measured with the methods described in Example 5.

### T Cell Response.

To determine if there is a T cell response induced by the EPL:allergen gene vaccination protocol, we will assess the key cytokine production that reflects characteristic Th1/Th2 and T regulatory responses. Animals will be sacrificed at days 0, 14, 28 and 42 as indicated and cells from spleen and lymph nodes harvested. The cultured cells will be pulsed with purified Ara h1 or Gal d1 protein (10 μg/ml) for 48 hours to induce memory T cell cytokine production (IL-4, IL-5, IL-10, IL-12, IL-13, TGF-β and IFN-γ) as measured by cytokine specific ELISA assays and the cytokine mRNA expression profiles by the quantitative real-time RT-PCR. We will also measure the frequencies of IL-4 (as a Th2 response indicator) and IFN-γ (as a Th1 response indicator) producing cells by Elispot assay as this provides a cell frequency as opposed to a total level of cytokine.

### Example 5

### EPL:Allergen Gene Vaccine can Efficiently Inhibit the Induction of an Allergen Specific Allergic Response

### Sensitization Via Oral Administration Resulting in Reactivity to Oral and Systemic Challenge.

We are fortunate that several reasonably well-characterized animal models for allergic reactivity to foods have been developed. Protocol 1 is based on the work of Li and Sampson (Li X M et al., J Allergy Clin Immunol 106:150, 2000). The mice will be sensitized intragastrically (i.g.) with the designed allergen [crude peanut extract (CPE) for peanut allergy, Gal d1 for egg white allergy and αCasein for milk allergy] plus cholera toxin (CT) as adjuvant. Cholera toxin has been shown to be a particularly potent adjuvant in mice for the induction of allergic responses associated with the mucosal immune system, and this protocol has been shown to induce not only allergic antibodies but also clinical reactivity to oral and systemic challenge, mimicking the food allergic response in humans. Animals will be i.g. challenged to induce the systemic anaphylaxis (Li X M et al., J Allergy Clin Immunol 106:150, 2000: Li X M, et al., J Allergy Clin Immunol 103:206, 1999).

We are aware that, in general, investigators have used C3H/HeJ mice for this type of experiment with peanut allergy, while our transgenic mice are of the Balb/c background. Balb/c mice are known to make robust allergic antibody responses and we have shown that they have airway hyper-reactivity and systemic allergic reactivity to allergen challenge. However, if we do encounter difficulties with the oral sensitization/challenge protocol, we can use the standard intraperitoneal (i.p.) sensitization protocol below (Protocol 2), which we know will induce sensitization and reactivity to systemic challenge in Balb/c mice (Adel-Patient K et al., Allergy 60:658, 2005; Rebecca J. Dearman and Ian Kimber. Methods 41:91-98. 2007). Alternatively, we can backcross onto the C3H/HeJ strain to provide for reactivity to oral challenge.

Protocol 2 Egg, Milk and Peanut Sensitization by i.p. Administration of Allergen with Alum Resulting in Reactivity to Systemic Challenge.

This protocol, an alternative, employs i.p. sensitization with alum as the adjuvant, which is a standard sensitization protocol in Balb/c mice that have been successfully employed in the food allergy, including peanut allergy model ((Adel-Patient K et al., Allergy 60:658, 2005; Rebecca J. Dearman and Ian Kimber. Methods 41:91-98. 2007). Human IgE+-hFcεRIα+ tg mice will be sensitized with egg, milk or peanut protein at day 0 and boosted i.p. on day 7 and 14. Allergen challenges will be performed 14 days after the last allergen treatment. In some experiments animals will receive a subsequent allergen booster to prolong their allergic reactivity so that the effects of treatment over a several week time period can be assessed without the spontaneous loss of allergic reactivity in the untreated controls. Importantly, we know that mice sensitized by this protocol to cat allergen remain sensitized and clinically reactive to the allergen if provided with an occasional allergen booster challenge (Terada T et al., Clin Immunol 120:45, 2006). This will allow sufficient time for testing the effects of our IgE-mediated DNA vaccination therapy in animals that maintain their allergic reactivity.

### Experimental Design and Methods

### i) Procedures

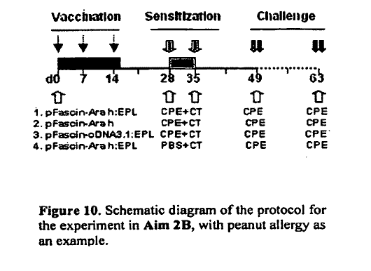

Human IgE⁺-hFcεRIα⁺ tg mice (4-6-week old, 8 mice/group) will be divided into 4 groups, as diagrammed in FIG. 10, and receive three i.d. vaccinations at days 0, 7, and 14, with EPL:pFascin-allergen (using Ara h as an example for peanut allergy shown in FIG. 10) or control EPL:pFascin-cDNA3 gene polyplex (10 μg plasmid DNA per mouse). The same group design applies to Gal d1 for egg allergy and αCasein for milk allergy. In the case of peanut allergy, the animals and controls will be i.g. sensitized with CPE (1 mg/mouse) plus CT (10 μg/mouse) in two doses at days 28 and 35, followed by oral challenge at day 49 with 10 mg CPE divided into two doses for the first challenge. The systemic anaphylaxis signs should appear about 15 minutes after the first dose of challenge, and the clinical indexes will be scored 30 minutes after the second dose of challenge. The mouse that survives this first challenge will be rechallenged at day 63 (FIG. 10). Since the first i.g. challenge also functions as booster sensitization, the rechallenge at day 63 generally should induce even stronger systemic anaphylaxis and allergic response. Following the vaccination, sensitization and challenge (and rechallenge), we will collect according to the schedule indicated in FIG. 10 blood samples to assess the allergen specific humoral and cellular immune/allergic responses and thereby to define the effects of IgE-mediated allergen gene vaccination on the allergen specific immune/allergic responses as compared to controls. The day 0 samples will serve as baseline, the day 28 samples will represent the immune responses induced by Ara h gene vaccination prior to allergen sensitization, the day 35 samples will reflect the modulation of the primary immune/allergic responses by DNA vaccination, and the days 49 and 63 samples will reflect the modulation of the secondary (or boosted) immune/allergic responses by DNA vaccination. The experimental results from group 1 will be compared with those of group 2 to determine the efficiency of the IgE-mediated allergen vaccination, and the results from group 3 (sham vaccinated and sensitized) and group 4 (vaccinated and sham sensitized) will serve as controls.

### ii) Modulation of the Peanut Allergic Response by a Single Ara h Gene Vaccination

In the first set of experiments as diagrammed in FIG. 10, we will test the effects of single allergen gene (using Ara h1 and Gal d1 as prototypes for peanut and egg allergy, respectively) vaccination on allergen specific immune/allergic responses. As this Ara h1 gene vaccination is expected to merely modulate the Ara h1 specific immune and/or allergic responses, the clinical indexes of the systemic anaphylaxis and allergic response of the CPE-induced peanut allergy are unlikely to be significantly modulated by the single Ara h1 gene vaccination; therefore, we will assess the parameters that reflect the allergen specific immune responses, especially IgE, IgG1 and IgG2a levels and cytokine expression profiles such as IL-4, IL-5, IL-10, IL13, IFN-β and TGF-γ that reflect the Th1/Th2 responses. In these and subsequent experiments, we will generally sensitize with the food, e.g. peanut extract (CPE) or egg white protein, rather than the specific gene product Ara h or Gal d1 protein as doing so has several advantages. Purified proteins, e.g. Ara h1 (and other Ara h) proteins by themselves are often not as potent as an immunogen/allergen as CPE for inducing peanut allergy sensitization (Van wijk F, Nierkens S, et al., Toxicol Sci 86:333, 200580). Second, the induction of immune/allergic responses to several allergens in food, e.g. Ara h proteins in CPE, will provide an internal antigen specificity control for the specific allergen gene treatment. As a result, allergen-specific immune modulation caused by IgE-mediated Ara h1 gene vaccination can be evaluated and compared to the predicted lack of effect on Ara h2, Ara h3 or Ara h6 responses. Furthermore, it will be possible to challenge animals for clinical reactivity with the individual allergens to again show allergen specificity. The same situation holds for Gal d1; in this case ovalbumin (Gal d2) will serve as a control.

### iii) Modulation of the Peanut Allergic Response by a Combined Ara h Gene Vaccination

The animals will be vaccinated with multiple allergen genes (the combined Ara h1, Ara h2, Ara h3 and Ara h6) polyplexes three times, followed by CPE-sensitization and CPE-challenge, according to the same schedule shown in FIG. 10. This set of experiments will allow us to determine not only the modulation of allergen specific antibody/cytokine responses, but also the clinical manifestation of the systemic anaphylaxis, because the combined Ara h1, Ara h2, Ara h3 and Ara h6 allergen presents the vast majority (over 90%) of all the allergens in peanut. Therefore this experiment will test whether a level of physiologic protection can be achieved by the combined Ara h gene vaccination.

We will see decreased sensitization as manifested by less human IgE and murine IgG1 to the relevant allergen and the production of a less Th2 biased cytokine profile in response to the allergen over the ensuing experimental period. Enough mice will be entered into each protocol so that groups of mice can be sacrificed for T cell response studies at the end of the sensitization and two and four weeks later. Mice not sacrificed at any time point will provide serum so that we have a series of sequential antibody measurements on individual mice. Additional controls will be obtained by vaccination of hIgE-hFcεRIαnegative littermates with EPL:Ara h1. Analogous controls will be used for the Gal d1 experiments.

### Potential Modifications of the Vaccination Protocol.

We will initially use three i.d. vaccinations of 10 μg of plasmid DNA per mouse given one week apart as a general standard for gene vaccination, as indicated in FIG. 10. However, unless we see a complete abolition of sensitization, we will modify the key parameters of (i), vaccine dose (1-50 ug plasmid DNA), (ii) timing of vaccinations, and (iii) number of vaccinations in subsequent experiments in order to define the maximum therapeutic benefit. Several routes, including intramuscular (i.m.), intradermal (i.d), or intraperitoneal (i.p.) injection, oral administration, or gene gun have been used for DNA vaccination. In addition to the planned i.d. administration, the i.m. and i.v. routes of administration are of particular interest as the former is a standard route of vaccination of humans and in gene therapy models, while the i.v. route is unexplored yet could provide a rapid-systemic form of vaccination. All three routes would be acceptable in humans.

i) Serum histamine levels and the core body temperature changes will be used as parameters for systemic anaphylaxis. The serum histamine level will be measured by ELISA kit, and the core body temperature will be measured with a rectal probe coupled to a digital thermometer, as described previously (Zhu C, et al., Nat Med 11:446, 2005; Terada T, et al., Clin Immunol 120:45, 2006).

ii) Systemic anaphylaxis assessment: Anaphylactic clinical index (symptoms) were evaluated 30-40 min after the second challenge dose using a scoring system as described by Li et al (J Allergy Clin Immunol 106:150, 2000): 0, no symptoms; 1, scratching and rubbing around the nose and head; 2, puffiness around the eyes and mouth, diarrhea, pilar erecti, reduced activity, and/or decreased activity with increased respiratory rate; 3, wheezing, labored respiration, cyanosis around the mouth and the tail; 4, no activity after prodding, or tremor and convulsion; and 5, death. Symptoms scoring will be performed in a blinded manner.

iii) Allergic vascular leakage: Immediately before the second dose of the intragastric peanut challenge, the mice from each group received 100 μL 0.5% Evan's blue dye by tail vein injection. Footpads of mice were examined for signs of vascular leakage (visible blue color) 30 to 40 minutes after dye/antigen administration as described (Li et al, J Allergy Clin Immunol 106:150, 2000).

iv) PCA assay will be used to functional determine the allergic reactions reflecting IgE-dependent allergic responses (60, 61, 69, 81). The Ara h gene vaccinated mouse serum will be serially diluted and sensitized by intradermal injection (50 μl) into the back skin. Twenty-four hours later, the mouse will be challenged through tail vein injection with 10 μg purified Ara h1 protein in the presence of 1% of Evan's blue dye in 200 μl saline solution. PCA is assessed visually as the blue dye staining of the skin 30 minutes post allergen challenge, and the diameter of the bluing spots will be measured and recorded for statistical analysis among the experimental groups. To demonstrate that IgE, instead of other components in the serum (such as IgG1), is responsible for the allergic reaction in PCA assay, the serum will be heat treated at 56° C. for 2 hours to inactivate IgE's activity prior to PCA test (Lyczak J B, et al., J Biol Chem 271:3428, 1996; Zhang K, et al., J Allergy Clin Immunol 114:321, 2004).

v) Mast cell degranulation: Mast cell degranulation during systemic anaphylaxis will be assessed by histologic examination of ear tissues (Lyczak J B, et al., J Biol Chem 271:3428, 1996). Samples collected immediately after anaphylaxis-related death or 40 min after challenge from surviving mice will be fixed and processed into 3 μm paraffin or glycol methacrylate, toluidine blue-stained sections. A degranulated mast cell is defined as a toluidine-positive cell with five or more distinct stained granules completely outside of the cell. A total of 200-400 mast cells will be classified in each ear sample.

### Antibody Outcome in Response to Ara h1 or Gal d1 Gene Vaccination:

Serum will be collected and the antibody level measured as scheduled in FIG. 10. We will measure peanut (Ara h) or egg allergen Gal d1 and Gal d2 (ovalbumin) specific human IgE and murine IgG1, IgG2a, and IgA antibodies by ELISA. As specific IgE levels represent a key parameter for evaluating the outcome of the gene vaccination, we will take particular care to assess the level of Ara h and Gal d1 specific human IgE. If necessary to improve the sensitivity and specificity of the IgE anti-Ara h (or Gal d1) assays due to the fact that high levels of murine IgG can compete with the IgE for the allergen in the ELISA format, we will remove murine IgG by absorption of the serum samples with protein G resin (Lehrer S B, et al., J. Immunol. Methods 284:1, 2004) or murine antibody reagents. The statistical significance for the experimental parameters described above will be compared.

The possible EPL left-over in vivo from the vaccination process will not compromise the accrual measurement of the allergen specific IgE produced in vivo in the hIgE-hFcεRIαtg mice, as the human IgE Fc portion of the EPL has no antigen (allergen) specificity and therefore is not expected to bind to the coated allergen, and therefore would not interfere the allergen-specific human IgE detection in the ELISA assay. To further ensure the accrual measurements for allergen specific human IgE, we will employ an anti-human IgE monoclonal antibody (Mae1) against the CH1 domain of IgE (Yamada, T., et al., J Biol Chem 278:32818-24, 2003, a kind gift from Dr. Paul Jardieu of Genentech Inc. CA) as the detective reagent in ELISA to confer the results, as the Fcε of EPL only contains epsilon CH2-CH3-CH4, but not CH1 (FIG. 4). In addition, PCA assay will also be used to functionally confer the IgE titers to corroborate the IgE measurements with that from ELISA (see PCA assay above).

### T Cell Outcome in Response to Ara h and Gal d1 Gene Vaccination:

The allergen-specific T cell changes driven by specific IT (conventional or otherwise) are thought to be an important mechanism for the induction and maintenance of allergic “tolerance”. We will assess the key cytokine production that reflects the characteristic Th1/Th2 responses and T regulatory responses.

Clinical Outcome in Animals Pretreated with Ara h or Gal d1 Gene Vaccination:

A standardized systemic anaphylaxis assessment using an anaphylactic clinical index will provide an overall evaluation of clinical reactivity. Serum histamine levels and core body temperature changes will be used as objective parameters of systemic allergic reactivity. Vascular leakage due to systemic reactivity following allergen challenge will be assessed by Evans blue staining of the footpad. Mast cell degranulation during systemic anaphylaxis will be assessed by histological examination of ear tissues, as described in the Method above.

### Example 6

### IgE-Mediated Allergen Gene Vaccines Will be Able to Treat an Established Allergic Disease

### i) Modulation of the Established Peanut Allergic Response by a Single Ara h Gene Vaccination:

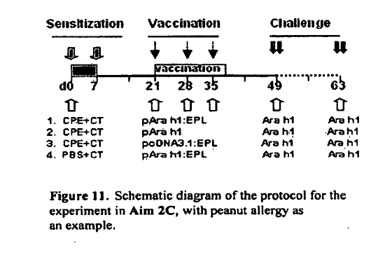

To test the ability of the IgE-mediated allergen gene vaccine to treat established allergic disease, we will sensitize the hIgE-hFcεRIαtg mice with food allergen (the same protocol will apply to peanut, egg or milk allergy testing). In the first set of experiments as diagrammed in FIG. 11, we will employ a single allergen gene vaccination protocol, e.g. Ara h1, to see if reactivity to a subsequent challenge with that allergen is modulated. Four groups of mice listed in the lower panel of the FIG. 11 will be i.g. sensitized with CPE plus CT twice at days 0 and 7, with the same protocol used in Example 5. Two weeks later (day 21), the animals will receive three weekly i.d. vaccinations of the EPL:pFascin Ara h1 polyplex (10 μg/mouse). The mice will be i.g. challenged with Ara h1 for the first time at day 49 and rechallenged at day 63, using the same protocol described in Aim 2B. The systemic anaphylaxis clinical manifestations will be scored with the method described in Example 5. The effects on Aha h1 antibody and T responses will be assessed. With the day 0 samples serving as baseline, the day 21 samples will represent the immune/allergic responses induced by CEP sensitization (with CT as adjuvant) prior to Ara h1 vaccination; the days 28, 35 and 49 blood samples will measure the modulation effects of first, second and third Ara h1 vaccination on CPE sensitization-induced immune/allergic responses, respectively; and the day 63 blood samples will measure the relatively long-term (one month after last vaccination) modulation effects of the immune/allergic responses by DNA vaccination. The day 49 i.g. challenge process will also function as boost sensitization for day 63 sample measurement. The experimental results from group 1 will determine the efficiency of the IgE-mediated allergen vaccination compared with that of conventional naked DNA vaccination (e.g. group 2), and group 3 will serve as vector control (CPE sensitized and sham vaccinated) and group 4 as non-sensitization (sham sensitized and Ara h1 vaccinated) control.

### ii) Modulation of the Established Peanut Allergic Response by a Combined Ara h Gene Vaccination:

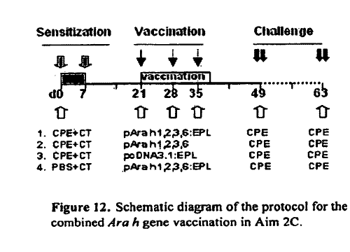

We will conduct a second set of experiments by employing a combined vaccination protocol, e.g. using combined A Ara h1, Ara h2, Ara h3, and Ara h6 gene vaccination to treat peanut allergy, as diagrammed in FIG. 12. Because in real life subjects are generally sensitized to more than one allergen, we will also undertake gene vaccination using a profile of the most relevant peanut allergy genes (Ara h1, Ara h2, Ara h3, and Ara h6) (82) to see if we can modify disease most analogous to the human situation. To do this, we will prepare EPL;Ara Ara h1, Ara h2, Ara h3, and Ara h6 polyplexes for vaccination. One of the advantages of this gene therapy approach is the ease by which these mixed polyplexes can be assembled. Thus, we will only need to prepare the four individual pFastin Ara h1, Ara h2, Ara h3, and Ara h6 plasmids and mix them in equal proportions with the EPL to assemble the combined vaccination polyplexes. Four groups of mice listed in the lower panel of the FIG. 12 will be i.g. sensitized with CPE plus CT twice at days 0 and 7, using the same protocol in Example 5. The mice will be i.d. vaccinated for three times at days 21, 28 and 35, with combined Ara h1, Ara h2, Ara h3, and Ara h6 gene complexed with EPL, followed by CPE challenge at day 49 (first challenge) and day 63 (rechallenge), with the methods described in Example 5. The immune/allergic responses, as well as the clinical manifestations of the systemic peanut anaphylaxis, will be determined with the methods described in Example 5. We expect that the animals sensitized to multiple allergens in whole peanut extract will potentially be protected from whole peanut challenge by the combined gene vaccination with the mixed Aha h polyplexes compared to animals receiving a single allergen gene.

Experiments analogous to those with the Ara h proteins can be carried out using an EPL:Gal d1 gene vaccine in hIgE⁺-hFcεRIα⁺ tg mice that have been made allergic to egg in which the dominant allergen is Gal d1.

While the present application has been described in the context of embodiments illustrated and described herein, the invention may be embodied in other specific ways or in other specific forms without departing from its spirit or essential characteristics. Therefore, the described embodiments are to be considered in all respects as illustrative and not restrictive. The scope of the invention is therefore indicated by the appended claims rather than the foregoing description, an all changes that come within the meaning and range of equivalency of the claims are to be embraced within their scope.

What is claimed is:

1. A vaccine comprising a nucleic acid encoding an allergen functionally connected to an IgE fragment capable of binding a native Fce receptor.

2. The vaccine of claim 1 wherein the nucleic acid is indirectly functionally connected to the IgE fragment.

3. The vaccine of claim 2 wherein nucleic acid is connected to the IgE fragment by a nucleic acid binding agent.

4. The vaccine of claim 3 wherein the nucleic acid binding agent is selected from the group consisting of poly-L-lysine, poly-L-arginine-lysine, spermine, spermidine, and polyethylimine polymer.

5. The vaccine of claim 3 wherein the IgE fragment is attached to the nucleic acid binding agent by a linkage selected from the group consisting of a covalent bond, a disulfide bond and an avidin/streptavidin linkage.

6. The vaccine of claim 5 wherein the IgE fragment is attached to the nucleic acid binding agent by a covalent bond.

7. The vaccine of claim 4 wherein the nucleic acid binding agent is poly-l-lysine.

8. The vaccine of claim 1 wherein the IgE fragment comprises the CH2-CH3-CH4 domains of IgE.

9. The vaccine of claim 8 wherein the IgE fragment comprises the CH1-CH2-CH3-CH4 domains of IgE.

10. The vaccine of claim 1 wherein the IgE fragment is human.

11. The vaccine of claim 1 wherein the nucleic acid encoding the allergen is operably linked to a dendritic cell promoter.

12. The vaccine of claim 11 wherein the dendritic cell promoter is the fascin promoter.

13. The vaccine of claim 1 wherein the nucleic acid comprises a vector.

14. The vaccine of claim 1 wherein the allergen is selected from the group of Table 1.

15. The vaccine of claim 14 wherein the allergen is Fel d1.

16. A pharmaceutical composition comprising a vaccine of claim 1 in admixture with a pharmaceutically acceptable ingredient.

17. An article of manufacture comprising a container, a vaccine of claim 1 within the container, and a label or package insert on or associated with the container.

18. The article of manufacture of claim 17 wherein said label or package insert comprises instructions for the treatment of an IgE-mediated biological response.

19. The article of manufacture of claim 18 wherein said biological response is an IgE-mediated hypersensitivity reaction.

20. The article of manufacture of claim 19 wherein said label or package insert contains instruction for the treatment of an IgE-mediated hypersensitivity reaction selected from the group consisting of asthma, allergic rhinitis, atopic dermatitis, severe food allergies, chronic urticaria, angioedema, and anaphylactic shock.

21. The article of manufacture of claim 17 wherein said label or package insert comprises instructions for the treatment of an infectious disease.

22. The article of manufacture of claim 18 wherein said infectious disease is a viral infection.

23. The article of manufacture of claim 17 wherein said label or package insert comprises instructions for the treatment of an autoimmune disease.

24. The article of manufacture of claim 17 wherein said label or package insert comprises instructions for the treatment of cancer.

25. A method for the prevention or treatment of a condition associated with an IgE-mediated biological response, comprising administering an effective amount of a vaccine of claim 1 to a subject in need.

26. The method of claim 21 wherein said subject is a human patient.

27. The method of claim 22 wherein said condition is an IgE-mediated hypersensitivity reaction.

28. The method of claim 23 wherein said condition is selected from the group consisting of asthma, allergic rhinitis, atopic dermatitis, severe food allergies, chronic urticaria, angioedema, and anaphylactic shock.

29. The method of claim 24 wherein said administration is prior to the onset of said biological response.

30. A method for the prevention or treatment of an infectious disease, comprising administering an effective amount of a vaccine of claim 1 to a subject in need.

31. The method of claim 30 wherein the infectious diseases is a viral infection.

32. The method of claim 30 wherein the subject is a human patient.

33. A method for the prevention or treatment of an autoimmune disease, comprising administering an effective amount of a vaccine of claim 1 to a subject in need.

34. The method of claim 33 wherein the subject is a human patient.

35. A method for the prevention or treatment of cancer, comprising administering an effective amount of a vaccine of claim 1 to a subject in need.

36. The method of claim 35 wherein the subject is a human patient.
